SEQUENCE LISTING (208 sequences)

SEQ ID NO: 1 - DNA, 3221 bp, Zea mays
  misc_feature (1547)..(1573): n is a, c, g, or t
agtggtacggtggggtcgcggagcttgccagcttggtggctgcaactcgccagcctgatggggcggcttcgcgcgtgctgccgtctccaccgcttcctcgcgccgccgccggcacaagtgccatcaccgggacaatctctggcccgcgggtccaacaccgctaccgctcacgtagcgcctttctcgaggcttctctcgtcggccgcggccgttgcgccccacgacacacgcgactcgggcctcggcggttcggcgtactgggcctggattcgcgcggcgaccgagtcggccccggctccttcgccgccccaggaggaggaggacgagggcccagcacgctacatccccgtcaaagcctacttcctctccaccaggtaagcacgcagcgccgctcccgcgcttgttcgataggtgtttgatgatttatcatcgaggaaacattcgggactttcgggtgctaccctactattctaaaaatgtccatgccaattggcactgcgaactaaaacgacgtgttttcaatttttcctcctcgcggtgacagcattgatctgaagagcatgcaagcggagcacgggaatgatattgtacctccgtcgacccgtttgcttaactacattgcgcttcggtactccgagttcccacctgagattatggttagttattacagtttagcgtcacttcttttttgacctgaaattcatatcaaaatgaacaaatctcagtcagctttggaccagttgcacatactttgtaacattgacctatgctacgaattgatttccttgtgattttgattgatatttatgcagaacattggagtgaaggataacagattttgctatcgctatgtggttgttttccagtatggttctgctgtgcttttcaacattgctgatcatgaagccgagtactatcttgatataattaggaagcatgcttcaggatggcttccagagatgagaaaagatggtatgctcactgctaaactgcgtcctgtttactgctttttactggcctccatttttgtgttccttaataatatttgcatggacagaaaacagtcagttgccttctgttatattcaaagatgctatttgttaactgtgatagaactcaacttaaagagaattgttgcatgcctagactgtgtgttttgtgcatttttgtttagcagaaataagttgctactgcttttcgtccactgtgacaaagcatatgccccctggaattgaactgtttgactctttgtaaagatgcaggtttagagtaagaccaacatcagctgatgccatttattttgcatgaacagattatgcagtggttgagaaaccatccttgacaacatggatgaaaggaggacttgattatatagttcttaaaagtttagatacagacgggattcgcataatttcaagcgttcttggtcaaagtatcgcccttgatcactatattcggcaggtactgaaactcaaagttaatgaacttatgtgttgtcacagtatcattaaagctttgtctaaaagttctgaacttcaaaattatcnnnnnnnnnnnnnnnnnnnnnnnnnnnttcactgaaattaatcgtgtcatggagaaaaccggtaactttaccatgcaaagaaaaaaactctttcaacttgtgggtaaggccaattctaatcttgcggatgttatcatcagacttggtctttttgacaggtatgtctactacactcaagtactatacttcctatttaggatggttccatttcttgtacaacatgtgtgaagttaaatattttatggatcaaagaatgtgtcaatcccatgtggaggattgtgaaggtgtgtgattcaatttgctagacattgcacgtctataagccactgttatgcgtacatttgatattcccttttggggttcacatgtgggttttgctgtttgtttggaaattcttccaatactttgaaaaaatattgatgattttgatcattgtacttagaaattagacatcactctatcactatatcacaatattaactatttttataagtattgtgtttgctatccttggagtatatcatattctcgttggcttggctgcatgcatccccatgtttctgctttgactgtcttgttgcttttatagtgcaattttatgtcttgattcctgcctattttttccttttcttttttgcttctcgatacattcacctactgtcatttggccatgttttttgctaggtcagaaattgcttggaaaaatgcaaattatgcacaaattctagaatatcttcgggaggaatatgaactgaatcagcgttttggaagccttgacttcaaactgaaatttgtggaggtagtgctttgtgtattcattctcctatagtgcaaaagttgggttctttatctttcaagttctcatttgcgcttacgatttattattgtaacagcataacattcactttcttcaagaggtcctccaaaacagacggtctgatctgctggaatggggtgtcataatactgctgattattgagattgctatctcattatatgaaattgccaaggactccatgatcgattgagagctctcaacaacacctctattcatcctcgatcatcccattatgctaactccagattactgttttaacgaaaaaaaggaaaagaatgaccccagcttccatattttctgactatccatcttcaccctttggatttctaaccttagatgaaaggaagttgggactggcgttctggcttgagtttatttacatcctttaattctacaactccaaacgaaaagaagtttggccaattttcctatgtccaatgagttcccgggcagccagggttttctaatttgagtagaatctagttttcctttcaatattcatgccaggtgttcttgatacatgttcttggtgccactttttagtatcttgataggactgtcagctctttttttgggggcaaggctgacgccaggcagctgcaactggacctgatggaaagatcatgaaagtttttatcaaatggctttgtaatttgtacaaaaacttagcgtgggaaggttccctttggtttaaccatactgaattgctcgttccattttgcgtggtaattaaccatgcatactgtaagtatgtgtagtacacttgatgcctatgagtgagagtgaattagatatttcttctagtcaaaatctatgcaataaataaactatctatgt

SEQ ID NO: 2 - DNA, 3214 bp, Zea mays
agtggtacggtggggtcgcggagcttgccagctttgtggctgcaactcgccagcctgatggggcggcttcgcgcgtgctgccgtctccaccgcttcctcgcgccgccgccggcacaagtgccatcaccgggacaatctctggcccgcgggtccaacaccgctaccgctcacgtagcgcctttctcgaggcttctctcgtcggccgcggccgttgcgccccacgacacacgcgactcgggcctcggcggttcggcgtactgggcctggattcgcgcggcgaccgagtcggccccggctccttcgccgccccaggaggaggaggacgagggcccagcacgctacatccccgtcaaagcctacttcctctccaccaggtaagcacgcagcgccgctcccgcgcttgctcgataggtgtttgatgatttatcatcgaggaaacattcgggtgctaccttactattctaaaaatgtccatgccaattggcactgcgaactaaaacgacgtgttttcaatttttcctcctcgccgtgacagcattgatctgaagagcatgcaagcggagcacgggaatgatattgtacctccgtcgacccgtttgcttaactacattgcgcttcggtactccgagttcccacctgagattatggttagttattacagtttagcgtcacttcttttttgacctgaaattcatatcaaaatgaacaaatctcagtcagctttgaaccagttgcacatagacctatgctacgaattgatttccttgtgattttggttgatatttatgcagaacattggagtgaaggataacagattttgctatcgctatgtggttgttttccagtatggttctgctgtgcttttcaacattgctgatcatgaagccgagtactatcttgatataattaggaagcatgcttcaggatggcttccagagatgagaaaagatggtatgctcactgctaaactgcgtcctgtttactgctttttactggcctccatttttgtgttccttaataatattttcatggaccgaaaacagtacagttgccttctcccttctgttatattcaaagatgctatttgttaactgtgatagaactcaacttaaagagaattgttgcatgcctagactgtgtgttttgtgcatttttgtttagcagaaataagttgttactgcttttcgtccactgtgacaaagcacatgccccctggaattgaactgtttgactctttgtaaagatgcaggtttagagtaagaccaacatcagctgatgccatttattttgcatgaacagattatgcagtggttgagaaaccatccttgacaacatggatgaaaggaggacttgattatatagttcttaaaagtttagatacagacgggattcgcataatttcaagcgttcttggtcaaagtatcgcccttgatcactatattcggcaggtactgaaactcaaagttaatgaacttatgtgttgtcacagtatcattaaagctttgtctaaaagttctgaacttcaaaattatcaattttgataggtcgatgatatggttgaggaattcactgaaattaatcgtgtcatggagaaaaccggtaactttaccatgcaaagaaaaaaactctttcaacttgtgggtaaggccaattctaatcttgcggatgttatcatcagacttggtctttttgacaggtatgtctactacactcaagtactatacttcctatttaggatggttccatttcttgtacagcatatgtgaagttaaatattttatggatcaaagaatgtgtcaatcccatgtggaggattgtgaaggtgtgtgattcaagttgctagacatagcacgtctataagccactgttatgcgtacatttgatattcccttttggggttcacatgtgggttttgctgtttgtttggaaattcttccaatactttgaaaaaatattgatgattttgatcattgtacttagaaattagacatcactctatcactatatcacaatattaactatttttataagtattgtgtttgctatccttggagtatatcatattcttgttggcttggctacatgcatccccatgtttctgctttgactgtcttgttgcttttatagtgcaattttacgtcttgattcctgcctatttttttccttttcttttttgcttctcgatacattcacctactgtcatttgtccatgttttttgctaggtcagaaattgcttggaaaaatgcaaattatgcacaaattctagaatatcttcgggaggaatatgaactgaatcagcgttttggaagccttgacttcaaactgaaatttgtggaggtagtgctttgtgtattcattctctatagtgcaaaagttgggttctttatctttcaagttctcatttgcgcttacgatttattattgtaacagcataacattcactttcttcaagaggtcctccaaaacagacggtctgatctgctggaatggggtgtcataatactgctgattattgagattgctatctcattatatgaaattgccaaggactccatgatcgattgagagctctgaacaacacctctattcatcctcgatcatcccattatgctaactccagatccactgttttaacaaaaaaaggaaaagaatgaccccagcttccatattttctgactatccatctccaccctttggatttctaaccttagatgaaaggaagttgggaatggcgttctggcttgagtttatttacatcctttaattctacaactccaaacgaaaagaagtttggccaattttcctatgtccaatgagttcccgggcagccaggttttccaatttgagtagaatctagttttcctttcaatattcatgccaggtgttcttgatacatgttcttggtgccactttttagtatcttgataggactgtcagctctttttttttgggggcaaggctgacgccaggcagctgcaactggacctgatggaaaaatcatgaaagtttttatcaaatggctttgtaatttgtacaaatacttagcgtgggaaggttccatttggtttaaccatactgaattgctcgttccattttgcgtggtaattaaccatgcatactgtaagtatgtgtagtacacttgatgcctatgagtgagagtgaattagatatttcttctagtcaaaatctatgcaataaataaactatctgtgt

SEQ ID NO: 3 - DNA, 1140 bp, Artificial Sequence
  cDNA of Zm00001d043358 (genotype B73)
atggggcggcttcgcgcgtgctgccgtctccaccgcttcctcgcgccgccgccggcacaagtgccatcaccgggacaatctctggcccgcgggtccaacaccgctaccgctcacgtagcgcctttctcgaggcttctctcgtcggccgcggccgttgcgccccacgacacacgcgactcgggcctcggcggttcggcgtactgggcctggattcgcgcggcgaccgagtcggccccggctccttcgccgccccaggaggaggaggacgagggcccagcacgctacatccccgtcaaagcctacttcctctccaccagcattgatctgaagagcatgcaagcggagcacgggaatgatattgtacctccgtcgacccgtttgcttaactacattgcgcttcggtactccgagttcccacctgagattatgaacattggagtgaaggataacagattttgctatcgctatgtggttgttttccagtatggttctgctgtgcttttcaacattgctgatcatgaagccgagtactatcttgatataattaggaagcatgcttcaggatggcttccagagatgagaaaagatgattatgcagtggttgagaaaccatccttgacaacatggatgaaaggaggacttgattatatagttcttaaaagtttagatacagacgggattcgcataatttcaagcgttcttggtcaaagtatcgcccttgatcactatattcggcaggtcgatgatatggttgaggaattcactgaaattaatcgtgtcatggagaaaaccggtaactttaccatgcaaagaaaaaaactctttcaacttgtgggtaaggccaattctaatcttgcggatgttatcatcagacttggtctttttgacaggtcagaaattgcttggaaaaatgcaaattatgcacaaattctagaatatcttcgggaggaatatgaactgaatcagcgttttggaagccttgacttcaaactgaaatttgtggagcataacattcactttcttcaagaggtcctccaaaacagacggtctgatctgctggaatggggtgtcataatactgctgattattgagattgctatctcattatatgaaattgccaaggactccatgatcgattga

SEQ ID NO: 4 - protein, 379 aa, Zea mays
MGRLRACCRLHRFLAPPPAQVPSPGQSLARGSNTATAHVAPFSRLLSSAAAVAPHDTRDSGLGGSAYWAWIRAATESAPAPSPPQEEEDEGPARYIPVKAYFLSTSIDLKSMQAEHGNDIVPPSTRLLNYIALRYSEFPPEIMNIGVKDNRFCYRYVVVFQYGSAVLFNIADHEAEYYLDIIRKHASGWLPEMRKDDYAVVEKPSLTTWMKGGLDYIVLKSLDTDGIRIISSVLGQSIALDHYIRQVDDMVEEFTEINRVMEKTGNFTMQRKKLFQLVGKANSNLADVIIRLGLFDRSEIAWKNANYAQILEYLREEYELNQRFGSLDFKLKFVEHNIHFLQEVLQNRRSDLLEWGVIILLIIEIAISLYEIAKDSMID

SEQ ID NO: 5 - DNA, 3168 bp, Zea mays
caaatcggtagtttcagttccttcacttgtctgcatcccccttcccctaactccgccgccactgccgtccgcgctcgccgtcgccccgaacctatcgcccactgcatttttccggcggaggtatgcgatgaggctgctgcaagcggaggagctatttcggaaggttctggaggtcgggtcgaagaataaggcggctcggctgctggggctcgacgtcggcagcaagtacgtcggggtggccatctccgatgagaagaacagggtcgctatgcctctgaggtaagcacctccaacaactgctgcccgcggcacactgtgctcgctgcgataagtgctatgtttagaggatttgattttcttcctatgttaacttgtgtactcactgtggtaatccaacataagctcatatgtgccatctcaagatgcagatgcaggttgaaatatgaatgtgtgtgcaaaaacgttttttggccaattggcctcctaaaattatttcagtccaaattttttttggcccgcttaaattgcagtggtaccaattcatatgtaaacttagtcaagatagtattcaatgattggatcgaggtgttttatttgtattctgcatattgaagcgacagaaatggtgtgaattgtcaggtggtagatgtggtgctatcctattatgatcttcttttggatattgtgtgtgcacctctgaattggtggggaaaggatatgcaacaacatcctatggaatgcatttggtctactcaaccactttatttgcatcaattgtttaaaactgcatgaataataaaaatgaaaagtacaaattatcatattggttgggtaaatggaagactccaatattctgtggaatttcatttctttacatatatatttgattcaaggaccacacgtgtactcatccctatgtgtagtagattagaaatagagatgcaggtactcagaaaacactgctagacatttaacagtacaagataagcatggctactgatccatctgtgtattcgttttttctcatgtataacatagatttggtaaactgtgcgagtctgttacacccgaccttcatccgtttcccctttgctcaatcaggctgggtcaagcaggtcagactatatatctgatagggtgttttagtctaaaccttttttctcttatcccttgtgtacttctctggagttttgtaataatttcctctgttgctgtcacacagttaagtctgagaagattcgaatatcttcctttccttgtcccttgtgtacttgcctggagttttgcaataattgcctctgttgctgtcacaaagttaagtctgagaagattcagattacaagcccaacaagataatttgttgttttatggatattggctatattcctaaaatctacttagtttctttatagccaaatctatatcttgtgaaaaaaatgtaacaactgcaattcagacgacagtttacattcatgtacttgttagaaaaggacatgtatcattatctgtggctaagatccttattgaatgaatcttgcagtgttttgtgtcggacaaaaacaaacatcaacttgatggcagatgatttcaaaacattggtatgttgaaaatacctgttcagtttctggtatcatataaattttggatatatcaaaagtcggtccttagattgagtactcaatatgctatacagcatcttttcataacgaatggttgtgcatcacaccatcattctcttttattaaattcattttggtttcatttattgccccgaaatatttaggtttggcagttggcactgcaatttatttgtagtaatgctaagttctatacgtagatcagcatttttaactgattggttgcagctctatagttgttgctttagtttatgtttaattggaatgaaagatgtaatcaccaatgccaactttgtttactgggataggtttcgatgtattccatagctgggttcgttgtgggctatccattcaaaatctacggtcaaccttgtgcaagtgtaaagtgcccatgctcatgtacaattcccgtttgttctacttaatcttttttttaatgttctcttttgtctgtaaatcaggcaatccaagtaaggcatcttgctggggaactttgtaaaacagggaaacttgatgatctgccctacgcatattgggatgaaaatttcacctcaaaggtgaactctgttttcttctcggaaatttcttgctgatacatgagaaacaggattaataggtggaaagtgcattttttctgctgaatgtggacgctacgctacgttacattacattgttgtaacatgctataggcatggtatcagattcgttaatagtgctgctccttctatgcacacgtttttactctcctcccatctctctttcaagttcttcatttcattagtggagctctttgattgatcatttgatctcctccttgtggttttccgcagtgtgtagaagccctcttgcatcccctgaatctaaaagacttggacgatgccaaaacaatgacagacaaatttgctgcagtctgtatactccaggtgcgtactcctaagtaccagggtgactttgcgttactgctgcagatttcatgtttttcaactcttcgtgacagatggtacatagtttattcagttaaagtcatctgaagctgctttagtgacactgacgacgatccattactgaacagggttatcttgacaacatgaacagaaaactgagatccacagataagtctgaagcataaaggtggcacctaagattccagtttgaactaaaccaaagagcaagggaaccttttttttttgatagaacaggaggggagggcccctactggaaattttatttaaaatttgaaaagaaaacaaagtttagaacaaacaagaagacaaattacagggggttaaggagagtttgtactgccattgttttctactgccattgttgaagccaagtcgggatggcagaatcaaatctatccaaagctctgtgaatgacaaggagcaatttatttgcaaactcctgcttgcaatgttgcaccgttggatctttgttttgaaaaagctaagcattgcggcactttcaaatgctccaagtcatttgaataataacctccatagagaatgggacattgagttgctgttttagg

SEQ ID NO: 6 - DNA, 3155 bp, Zea mays
caaatcggtagtttcagttccttcacttgtctgcatcccccttcccctaactccgccgccactgccgtccgcgctcgccgtcgccccgaacctatcgcccactgcatttttccggcggaggtatgcgatgaggctgctgcaagcggaggagctatttcggaaggttctggaggtcgggtcgaagaataaggcggctcggctgctggggctcgacgtcggcagcaagtacgtcggggtggccatctccgatgagaagaacagggtcgctatgcctctgaggtaagcacctccaacaactgctgcccgcggcacactgtgctcgctgcgataagtgctatgtttagaggatttgattttcttcctatgttaacttgtgtactcactgtggtaatccaacataagctcatatgtgccatctcaagatgcagatgcaggttgaaatatgaatgtgtgtgcaaaaacgttttttggccaattggcctccttaaattatttcagtccaaattttttttggcccgcttaaattgcagtggtaccaattcatatgtaaacttagtcaagatagtattcaatgattggatcgaggtgttttatttgtattctgcatattaaagcgacagaaatggtgtgaattgtcgggtggtagatgtggtgctatcctattatgatcttcttttggatattgtgtatgcacctctgaattggtggggaaaggatatgcaacaacatcctatggaatgcatttggtctactcaaccactttatttgcatcaattgtttaaaactgcatgaataataaaaatgaaaagtataaattatcatattggttgggtaaatggaagactccaatattctgtggaatttgatttctttacatatatatttgattcaaggaccacaagtgtactcatccctatgtgtagtagattagaaatagagatgcaggtactcagaaaacactgctagacatttaacagtacaagataaacatggctactgatccatctgtgtatttgttttttctcatgtataacatagatttggtaaactgtgcgagtctgttacacccgaccttcatccgttttccctttgctcaatcaggctgggtcaagcaggtcagactatatatctgatagggtgttttagtctaaaccttttttctcttatcccttgtgtacttctctggagttttgtaacaatttcctctgttgctgtcacacagttaagtctgagaagattcgaatatcttcctttccttgtcccttgtgtacttgcctggagttttgcaataattgcctctgttgctgtcacaaagttaagtctgagaagattcagattacaagctcaacaagataatttgttgttttatggatattggctatattcctaaaatctacttagtttctttatagccaaatctatatcttgtgaaaaaaatgtaacaactgcaattcagacgatagtttacattcatgtacttgttagaaaaggacatgtatcattatctgtggctaagatccttattgaatgaatcttgcagtgttttgtgtcggacaaaaacaaacatcaacttgatggcagatgatttcaaaacattggtatgttgaaaatacctgttcagtttctggtatcatataaattttggatatatcaaaagtcggtccttagattgagtactcaatatgctatacagcatcttttcataacgaatggttgtgcatcacaccatcattctcttttattaaattcattttggtttcatttattgccccgaaatatttaggtttggcagttggcactgcaatttatttgtagtaatgctaagttctatacgtagatcagcatttttaactgattggttgcagctctatagttgttgctttagtttatgtttaattggaatgaaagatgtaatcaccaatgccaactttgtttattgggataggtttcgatgtattccatagctgggttcgttgtgggctatccattcaaaatctatggtcaaccttgtgcaagtgtaaagtgcccatgctcatgtacaattcccgtttgttctacttaatcttttttttaatgttctcttttgtctgtaaatcaggcaatccaagtaagtcttgctggggaactttgtaaaacagggaaacttgatgatctgccctacgcatattgggatgaaaatttcacctcaaaggtgaactctgttttcttctcggaaatttcttgctgatacatgagaaacaggattaataggtggaaagtgcattttttctgctgaatgtggatgctacgttacattacattgttgtaacatgctataggcatggtatcagattcgttaatagtgctgctccttctatgcacacgtttttactctcctcccatctctctttgaagttcttcatttcattagtggagctctttgattgatcatttgatctccttgtggttttccgcagtgtgtagaagccctcttgcatcccctgaatctaaaagacttggacgatgccaaaacaatgacagacaaatttgctgcagtctgtatactccaggtgcgtactcctaagtaccagggtgactttgcgttactgctgcagatttcatgtttttcaactcttcgtgacagatggtacatagtttattcagttaaagtcatctgaagctgctttagtgacactgacgacgatccattactgaacagggttatcttgacaacatgaacagaaaactgagatccacagataagtctgaagcataaaggtggcacctaagattccagtttgaactaaaccaaagagcaagggaaccttttttttgatagaacaggaggggagggcccctactgaaaattttatttaaaatttgaaaagaaaacaaagtttagaacaaacaagaagacaaattacagggggttagggagagtttgtactgccattgttttctactgccattgttgaagccaagtcgggatggcagaatcaaatctacccaaagctctgtgaatgacaaggagcaattcatttgcaaactcctgcttgcaatgttgcaccgttggatctttgttttgaaaaagccaagcattgcggcacttccaaatgctcaaagtcatgagaataataacctccatagagaatgggacattgagttgctgttttagg

SEQ ID NO: 7 - DNA, 525 bp, Artificial Sequence
  cDNA of Zm00001d043352 (genotype B73)
atgaggctgctgcaagcggaggagctatttcggaaggttctggaggtcgggtcgaagaataaggcggctcggctgctggggctcgacgtcggcagcaagtacgtcggggtggccatctccgatgagaagaacagggtcgctatgcctctgagtgttttgtgtcggacaaaaacaaacatcaacttgatggcagatgatttcaaaacattggtttcgatgtattccatagctgggttcgttgtgggctatccattcaaaatctatggtcaaccttgtgcaagtgcaatccaagtaagtcttgctggggaactttgtaaaacagggaaacttgatgatctgccctacgcatattgggatgaaaatttcacctcaaagtgtgtagaagccctcttgcatcccctgaatctaaaagacttggacgatgccaaaacaatgacagacaaatttgctgcagtctgtatactccagggttatcttgacaacatgaacagaaaactgagatccacagataagtctgaagcataa

SEQ ID NO: 8 - protein, 174 aa, Zea mays
MRLLQAEELFRKVLEVGSKNKAARLLGLDVGSKYVGVAISDEKNRVAMPLSVLCRTKTNINLMADDFKTLVSMYSIAGFVVGYPFKIYGQPCASAIQVSLAGELCKTGKLDDLPYAYWDENFTSKCVEALLHPLNLKDLDDAKTMTDKFAAVCILQGYLDNMNRKLRSTDKSEA

SEQ ID NO: 9 - DNA, 3645 bp, Zea mays
  misc_feature (1562)..(1567): n is a, c, g, or t
aacacgctctcagtctcactcccacggtcccactcctcaggagcgtccagagtttaggtgctcgtggagtcgtggtggaggtggtaggtgtcgatggccttcaagctggccaccaaggccgcggcggcgtcgcccgctgctgctcaccgcgggggtctcgcccgggggccggagggtacgagccgcgttgccttcggaccagcgcctagaaacaaggggctccgcgcggccaacaactccgcgacgcccgtggctaagtacgaacgctccttctcctggttagaaattgtttaatttgataaacgactatgacccttggttccgcgtaaggtatggggggctggccctacatcagactttgtgtttgtgcgtggtcgtgatcgtggtgtggtgtggtgtggtaactcttatctaaagagatttaggattgggtcggttctcagttggggatctgtttggtgcgcttgagcccttctgaacttgatcatccatgtttttgatacttaaggcatccaatttgtagctactgctctagttctaggatttggagaactccttgtcgaattgtcccacagagctaatgattgtgctaacaaaatgacatgatgctgattgttctacgcctcttgcttaataccctgtgaagtttaattatttggtggtacacttgaccttggaagtcatgacctcccaaaagcatggtgtagcatgtaggtactatatatctaggttgttgcaactaaattgtggaaatttccacacctctgatggctcatatgttgtcattcctttgtgacgaagtcttcttgatttttgtgaatatattccagatacgaaagtggaaccgtggggaggaaattgacatggatggctatatatattaaccttgttgctttttgcacaaaggcttactgtcttatgtgatgctggattactttgtttttacgaaacccgctttagttatactgaaggaactgatcatttctttatcttaatggtgcacttttattagggaagagagggttgatcgaagtgaaatattgacattggatagcattagacaagttttgattagactagaagacagcatcatattcggccttttggagagagcacagttttgttacaacgctgatacatatgatagcaatgctttccacatggatggttttggcggctctttggttgaatatatggttagagaaactgaaaagctccatgcacaggtggaattcatttggacattttttaagaacaaaaacagttgatcatggcatgcaaaagaataacatacactattgtttttaacataggttgggagatacaagagcccagatgagcaccctttctttcctgaggatctgcctgagccccggttgccacctatgcagtacccaagggtaatgcatgttcttggttctcaaatgcttctcatgatgactgcttttgtttctttgtcactgtaatagatataaaccatcatttgcaatctaatgtaccattttcttcatggaggtagataaaagaaaatgtgcttttggatacagttgtttagcatccgnnnnnncaatatattgagcaatgctcagttagtgaacaacatataggtagtgctctgtgacttgcagaatgtactatcattattgcaaatgccacatcccacagaccttagatacaagtgacggaaagttctgtgtttactactatatattctactttctccaatatattttggaacaagctgtacagctctttattgtattgcgttgagtcaaaataagtgatggggttatttttttattacaacatatgctattttgactttgagtaaatccaaaagctcctattgaaccttaggtggagcagaggtgaaagtaacattatttgcattttttcaaggggatatccaattgatgatgatttactgctttgcttcaatttaatcgttggatggtttgacctgcatgaatcttgtcatattctacaagcgctaacatagtgccatcctaatccatgtgcaacatgaaatatttgtttatgctgcatttggactatcatgtgttgcaagattcataaatcataatgccaacaagttgtcctggtattatgactatgttagtacattttagatggattcatacttcgctcttgtcctttgcataggttttgcatcccattgccgattctatcaatatcaacaaagagatttggaaaatgtattttgatgaacttcttccaagattggtgaaaaaaggaagtgatggtaatgctggatccagtgctctttgtgacacgacctgcttgcaggtacagatgatattcatgttaaatcctacactcagttttatggtctatttggttcctttagtccagggactaaagttgtccctaccctgtttggttccaaggactaaaagtattcaaaacacattaaatgactataagaataccgaaatgccccttaccgttctaccgtctttagttcaactgaaataaatgatggacaaaaggtagaattaatatgttttagtcctttttagtcaccccttgaggggctagggactaaaaggactaatctttaatccctggaaccaaacagacccttagttctttgggtgcacatccgcttttgttccagacttccagtcaactaggtgtccaggttataaaattccttttgctctgtctgccacaattttcattcagttaaaagaatatatttgagcaaacaaagccagatcaaatttgcatgaatcaatccatgcaagattattttcatagtactggcctgatcattgctggtaacttgtgcaattttgcatttttataattccctttctctgtttttcttgttccatactccaggcgctctccaaaaggatccactatgggaagtttgtggcagaggctaagtttcaggagtccccggaagcttacatgccagccataatagctcaggtatgtgcttttgtttcaactctagtatttctagtacaactatttttcatttttcttttgttttatttcagagtttcataacaaaatcatatttcgattcatttgaagaaaaaaactgtcgatttcttgtcacgttttgcaaaatccaggaccgtgatcaactcatgcaccttctcacatatgaaacggtggagcgtgctatcgaacacagggtggaagccaaagccaagatcttcgggcaagaggtgaacatcggtgtggaggacaacggcagcccaccggtgtacaagatcgttccgagcttggtcgccgagctgtacagctacagaatcatgccgctaaccaaagaggttcaaatcgcgtatttgcttaggaggctggattgattgtgtttatgtagatgtaaattgccagatcagaactcctggcgttaagccagaacattggtaaataccggttttggtgtcaggatgcaaaatgtaccatgtgtggaattgtgccaacaaacaagcataatatcatgtggagttgtcgcgtagtcgcacctcgtgcgtactgccaatggacaaaggttgtagactgtatggaatgatggttcactttctttggcggatgcccttttgaatgggaataaaattctctcattgtcgttgaaatttatacagatccgctcacgtgccatcgaaa

SEQ ID NO: 10 - DNA, 3745 bp, Zea mays
aacacgctctcagtctcactcccacggtcccactcctcaggagcgtccagagtttaggtgctcgtggagtcgtggtggaggtggtaggtgtcgatggccttcaagctggccaccaaggccgcggcggcgtcgcccgctgctgctcaccgcgggggtctcgcccgggggccggagggtacgagccgcgttgccttcggaccagcgcctagaaacaaggggctccgcgcggccaacaactccgcgacgcccgtggctaagtacgaacgctccttctcctggttagaaattgtttaatttgataaacgactatgacccttggttccgcgtaaggtatggggggctggccctacatcagactttgtgtttgtgcgtggtcgtgatcgtggtgtggtgtggtgtggtgtggtaactcttatctaaagagatttaggattgggtcggttctcagttggggatctgtttggtgcgcttgagcccttctgaacttgatcatccatgtttttgatacttaaggcatccaatttgtagctactgctctagttctaggatttggaaaactccttgtcgaattgtcccacagagctaatgattgtgcgaacaaaatgacatgatgctgattgttctacgcctcttgcttaataccctgtgaagtttaattatttggtggtacacttgaccttggaagtcatgacctcccaaaagcatggtgtagcatgtaggtactatatatctaggttgttgcaactaaattgtggaaatttccacacctctgatggctcatatgttgtcattcctttgtgacgaagtcttcttgatttttgtgaatatattccagatacgaaagtggaaccgtggggaggaaattgacatggatggctatatatattaactctgttgctttttgcacaaaggcttactgtcttatgtgatgctggattgctttgtttttacgaaacccgctctagttatactgaaggaactgatcatttctttatcttaatggtgcacttttattagggaagagagggttgatcgaagtgaaatattgacattggatagcattagacaagttttgattagactagaagacagcatcatatttggccttttggagagagcacagttttgttacaatgctgatacatatgatagcaatgctttccacatggatggttttggcggctctttggttgaatatatggttagagaaactgaaaagctccatgcacaggtggaattcatttggacattttttaagaacaaaaacagttgatcatggcatgcaaaagaataacatacgctattgtttttaacataggttgggagatacaagagcccagatgagcaccctttctttcctgaggatctgcctgagccccggttgccacctatgcagtacccaagggtaatgcatgttcttggttctcaaatgcttctcatgatgactgcttttgtttctttgtcaccgtaagagatataaaccatcatttgcaatctaatgtaccattttcttcatggaggtagataaaagaaaatgtgcttttgaatacagttgtttagcatccgaattaaaaggcttgtacagccaatatattgagcaattctcagttagtgaacaacatataggtagtgttctgtgacttgcagaatgtactatcattattgcaaatgccacatcccacagaccttagatacaagtgacggaaagttctgtgtttactactatatattttactttctccaatatattttggaacaagctgtacagctctttattgtattgcgttgagtcgaaataagtgatggggttattttttttgtttttttattacaacatatgctcttttgactttgagtaaatccaaaagctcctattgaaccttaggtggagcagaggtgaaagtaacattatttgcatttttttcaaggggatatccatttgatgatgatttactgctttgcttcaatttaatcgttggatggtttgacactttgacctgcatgaatcttgtcatattctacaagcgctaacatagcgccatcctaatccatgtgcaacatgaaatatttgtttatgctgcatttggactatcatgcgttgcaagattcataaatcataatgccaacaagttgtcctggtattatgactatgttagtacattttagatggattcatacttcgctcttgtcctttgcataggttttgcatcccattgctgattctatcaatatcaacaaagagatttggaaaatgtattttgatgaacttcttccaagattggtgaaaaaaggaagtgatggtaatgctggatccagtgctctttgtgacacgacctgcttgcaggtacagatgatattcatgttaaatcctacactcagttttatggtctgtttggttcctttagtcctgggactaaagttgtccctaccaacaggttccaaggactaaaagtattcaaaacgcattaaatgactttataagaataccgaaatgccccttaccgttctaccgtctttagttcaactgaaataaatgatggacaaaaggtagaattaatatgttttagtcctttttagtcaccccttgaggggctagggactaaaataatttagtctttgttttagttccactgtttgtcaatttagggactaaatgtgattaaaatagaaggactaatctttaatccctggaaccaaacagacccttagttctttgggtgcacatccacttttgttccagacttccagtcaactaggtgtccaggttataaaactccttttgctctgtctgccacagttttcattcagttaaaagaatatatttgagcaaacaaagccagatcaaatttgcatgaatcaatccatgcaagattattttcatagtactggcctgatcattgctggtaacttgtgcaattttgcatttttataattccctttctctgtttttcttgttccatactccaggcgctctccaaaaggatccactatgggaagtttgtggcagaggctaagtttcaggagtccccggaagcttacatgccagccataatagctcaggtctgtgcttttgtttcaactctagtatttctagtacaactatttttcatttttttgttttatttcagagtttcataacaaaaatcatatttcgattcatttgaataaaaaaactgtcgatttcttgtcacgttttgcaaaatccaggaccgtgatcaactcatgcaccttctcacatatgaaacggtggagcgtgctatcgaacatagggtggaagccaaagccaagatcttcgggcaagaggtgaacatcggtgtggaggacaacggcagcccaccggtgtacaagatcgttccgagcttggtcgccgagctgtacagctacagaatcatgccgctaaccaaagaggttcaaatcgcgtatttgcttaggaggctggattgagtatgtttatgtagatgtaaattgccagatcagaactcctggcgttaagccagaacattggtaaataccggttttggtgtcaggatgcaaaatgtaccatgtgtggaattgtgccaacaaacaagcataatatcatgtggagttgtcgagtagtcgcgcctcgtgcgtactgccaatggacaatgtttgtagactgtatggaatgatgggtcacttcctttggcgggtgcccttttgaaagggagtaaaattctctcattgtcgttgaaatttatacagatccgctcaagtgccatcgaaa

SEQ ID NO: 11 - DNA, 939 bp, Artificial Sequence
  cDNA of Zm00001d043356 (genotype B73)
atggccttcaagctggccaccaaggccgcggcggcgtcgcccgctgctgctcaccgcgggggtctcgcccgggggccggagggtacgagccgcgttgccttcggaccagcgcctagaaacaaggggctccgcgcggccaacaactccgcgacgcccgtggctaaggaagagagggttgatcgaagtgaaatattgacattggatagcattagacaagttttgattagactagaagacagcatcatatttggccttttggagagagcacagttttgttacaatgctgatacatatgatagcaatgctttccacatggatggttttggcggctctttggttgaatatatggttagagaaactgaaaagctccatgcacaggttgggagatacaagagcccagatgagcaccctttctttcctgaggatctgcctgagccccggttgccacctatgcagtacccaagggttttgcatcccattgctgattctatcaatatcaacaaagagatttggaaaatgtattttgatgaacttcttccaagattggtgaaaaaaggaagtgatggtaatgctggatccagtgctctttgtgacacgacctgcttgcaggcgctctccaaaaggatccactatgggaagtttgtggcagaggctaagtttcaggagtccccggaagcttacatgccagccataatagctcaggaccgtgatcaactcatgcaccttctcacatatgaaacggtggagcgtgctatcgaacatagggtggaagccaaagccaagatcttcgggcaagaggtgaacatcggtgtggaggacaacggcagcccaccggtgtacaagatcgttccgagcttggtcgccgagctgtacagctacagaatcatgccgctaaccaaagaggttcaaatcgcgtatttgcttaggaggctggattga

SEQ ID NO: 12 - protein, 312 aa, Zea mays
MAFKLATKAAAASPAAAHRGGLARGPEGTSRVAFGPAPRNKGLRAANNSATPVAKEERVDRSEILTLDSIRQVLIRLEDSIIFGLLERAQFCYNADTYDSNAFHMDGFGGSLVEYMVRETEKLHAQVGRYKSPDEHPFFPEDLPEPRLPPMQYPRVLHPIADSININKEIWKMYFDELLPRLVKKGSDGNAGSSALCDTTCLQALSKRIHYGKFVAEAKFQESPEAYMPAIIAQDRDQLMHLLTYETVERAIEHRVEAKAKIFGQEVNIGVEDNGSPPVYKIVPSLVAELYSYRIMPLTKEVQIAYLLRRLD

SEQ ID NO: 13 - DNA, 812 bp, Zea mays
agcacaaaaatcaattgaacgttttgagaacgaaaataacacccgccgatactataccataaataaagttcaagaaatccacccctgcccgccgccgcctcttccttccggccggtcttcgtttcgacatccaggaaatggctaccgcggcgcgcctcctcctcgtcgtggtgcttctctccgcggcgataccgatcccgtccttcgcgacaagcttcgtcgtcggcgacaagaggcatcgctgggctcccaacgtcaactatacagactgggcggaccgtcaccagttccacgtcggcgactggctcggtatcattctcctcccctatatctatctacctttacatactcatcaacatcaaacagcccgtgtgatggagatctagctttcggcaaaagaattgatctaagggtgggaagacgcgttaatttgcacgcatgcagaattcaggtacgagagggacaggttcgatgtggtgcaggtgaacgagacggcatacgcggcgtgcgacgccagcagccccatcctcagctacagccgcggccacaacttcgtcttccggctcaaccacacgggccggttctacttcatctgcagccgcggctactgctggagtggcatgaaggtctccgtgctcgtccagccgccgccgcccccgtcgctgccgccggcgtcccactcccacgcgtcaagcgccagtgcgcgtgtgcggccggcggccgggctttggtgtgcggctctctctaccttgctgggctgggcggtgctcacgccgccgctgtcgtttcgggtctggagttctggatgtcca

SEQ ID NO: 14 - DNA, 915 bp, Zea mays
agcacaaaaatcaattgaacgttttgagaacgaaaataacacccgccgatactataccataaataaagttcaagaaatccacccctgcccgccgccgcctcttccttccggccggtcttcgtttcgacatccaggaaatggctaccgcggcgcgcctcctcctcgtcgtggtgcttctctccgcggcgataccgatcccgtccttcgcgacaagcttcgtcgtcggcgacaagaggcatcgctgggctcccaacgtcaactatacagactgggcggaccgtcaccagttccacgtcggcgactggctcggtatcattctcctcccctatatctatctacctttacatactcatcaacatcaaacagcccgtgtgatggagatctagctttcggcaaaagaattgatctaagggtgggaagacgcgttaatttgcacgcatgcagaattcaggtacgagagggacaggttcgatgtggtgcaggtgaacgagacggcatacgcggcgtgcgacgccagcagccccatcctcagctacagccgcggccacaacttcgtcttccggctcaaccacacgggccggttctacttcatctgcagccgcggctactgctggagtggcatgaaggtctccgtgctcgtccagccgccgccgccgcccccgtcgctgccgccggcgtcccactcccacgcgtcaagcgccagtgcgcgtgtgcggccggcggccgggctttggtgtgcggctctctctaccttgctgggctgggcggtgctcacgccgccgctgtcgtttcgggtctggagttctggacgtccaggcgcgcacacgagtctaaactctaaagctgtttgctaatgtactacccatttcttctttttttagtatggaaataagagttccggctttagcataaaga

SEQ ID NO: 15 - DNA, 582 bp, Artificial Sequence
  cDNA of Zm00001d043357 (genotype B73)
atggctaccgcggcgcgcctcctcctcgtcgtggtgcttctctccgcggcgataccgatcccgtccttcgcgacaagcttcgtcgtcggcgacaagaggcatcgctgggctcccaacgtcaactatacagactgggcggaccgtcaccagttccacgtcggcgactggctcgaattcaggtacgagagggacaggttcgatgtggtgcaggtgaacgagacggcatacgcggcgtgcgacgccagcagccccatcctcagctacagccgcggccacaacttcgtcttccggctcaaccacacgggccggttctacttcatctgcagccgcggctactgctggagtggcatgaaggtctccgtgctcgtccagccgccgccgccgcccccgtcgctgccgccggcgtcccactcccacgcgtcaagcgccagtgcgcgtgtgcggccggcggccgggctttggtgtgcggctctctctaccttgctgggctgggcggtgctcacgccgccgctgtcgtttcgggtctggagttctggacgtccaggcgcgcacacgagtctaaactctaaagctgtttgctaa

SEQ ID NO: 16 - protein, 193 aa, Zea mays
MATAARLLLVVVLLSAAIPIPSFATSFVVGDKRHRWAPNVNYTDWADRHQFHVGDWLEFRYERDRFDVVQVNETAYAACDASSPILSYSRGHNFVFRLNHTGRFYFICSRGYCWSGMKVSVLVQPPPPPPSLPPASHSHASSASARVRPAAGLWCAALSTLLGWAVLTPPLSFRVWSSGRPGAHTSLNSKAVC

SEQ ID NO: 17 - DNA, 71 bp, Artificial Sequence
  marker ma0016fm86@36
acatcttcttgccagagccgttggggtcgagggggrcgatcttcattgcttccgccggcttgaagttgccg

SEQ ID NO: 18 - DNA, 71 bp, Artificial Sequence
  marker ma0016fn05@36
aagtactataagggatgtcaagtgtgtcagaagttyagtgatctacaatttttttggtctctgaacatcat

SEQ ID NO: 19 - DNA, 71 bp, Artificial Sequence
  marker ma0016fn29@36
acctaggaaggtcagacggtctgaccatgtgccctrgatggtccataccccggatagtccacgagatagcc

SEQ ID NO: 20 - DNA, 71 bp, Artificial Sequence
  marker ma0016fn27@36
caccaccgcatagagcaccaccagaagatacaatcsctagtgtttggcccgaatcgaaaccaacagtgctc

SEQ ID NO: 21 - DNA, 71 bp, Artificial Sequence
  marker ma0016fn16@36
cagttcgcaagtagtcaaattatagaaaggtgagarcgcatgataataactttttcgagtatatccatttg

SEQ ID NO: 22 - DNA, 47 bp, Artificial Sequence
  marker ma0004tk22@12
cattacttggarttcagtgataaagttcatcataaattaaaacatac

SEQ ID NO: 23 - DNA, 71 bp, Artificial Sequence
  marker ma0004tk05@36
gaaacaagtcccctctgtaaggttctacccttgaamtataaaagagataggtccccctccttcgttccaca

SEQ ID NO: 24 - DNA, 71 bp, Artificial Sequence
  marker ma0016fn55@36
agataaataattcacataacattagcggttataagygagacgaagtaatagtgtgagacgaaaatggccac

SEQ ID NO: 25 - DNA, 71 bp, Artificial Sequence
  marker ma0016fp29@36
actccctctagtaagtgagtcttacataactagctyatcgttgtacagtgtacttgcatgtgcacaatgat

SEQ ID NO: 26 - DNA, 69 bp, Artificial Sequence
  marker ma0004tm29@36
ctagaattaaggatgataattttgaatagttaactraagaaacggtttaatagtattttctctacaata

SEQ ID NO: 27 - DNA, 71 bp, Artificial Sequence
  marker ma0004tm38@36
gaacttgtatcaggcaggtcctgtggaagagtatgygaagaattttgaggaggcatgttgtgcaacggcag

SEQ ID NO: 28 - DNA, 48 bp, Artificial Sequence
  marker ma0016fq46@15
aatacaagtttttgyatatttcccttcgctcggcatgatggtctgaac

SEQ ID NO: 29 - DNA, 71 bp, Artificial Sequence
  marker ma0000ba98@36
aaattggaacttttggggagtcacatactctcttamtgttagatgtctttctgatggctgtgatatcaaat

SEQ ID NO: 30 - DNA, 71 bp, Artificial Sequence
  marker ma0000na46@36
gtgatgtgcccacctctagcacctgagggattgaarcctgttactggcatggatctctgagcaaaatcaag

SEQ ID NO: 31 - DNA, 71 bp, Artificial Sequence
  marker ma0001gr36@36
gaatgctgcattcttaagattgattcgatctcttcrgatgacatgtacttggatctaaactttatacctcc

SEQ ID NO: 32 - DNA, 71 bp, Artificial Sequence
  marker ma0000kr37@36
taccagtatcagatggtagttgagagaatcgaatgytatgttttcccttccccgaccttgatctcttgtct

SEQ ID NO: 33 - DNA, 71 bp, Artificial Sequence
  marker ma0001gr37@36
tgatgaatgggaactgccaacagaaccaaacctgcytgtaatctctgcggagtgatggggatataggacgt

SEQ ID NO: 34 - DNA, 71 bp, Artificial Sequence
  marker ma0001gr41@36
ctctgctgatgctgagaaaataggagtgacgacatygggctattaggaagtgataattgtttgtgcaataa

SEQ ID NO: 35 - DNA, 52 bp, Artificial Sequence
  marker ma0016fq61@17
aatatcatccttaaaawatgttcaattgccacagcctaggaatataggggca

SEQ ID NO: 36 - DNA, 64 bp, Artificial Sequence
  marker ma0022xj91@29
aattcaacaaaaggatgtgtgcaagcatrcttgtcttccatctctgtcgtgcataactcaatcc

SEQ ID NO: 37 - DNA, 71 bp, Artificial Sequence
  marker ma0016fq64@36
cataactcaatccaataacgaagcgcgtgtgctacmaaacaaacgtataaccatttattccacacatctag

SEQ ID NO: 38 - DNA, 71 bp, Artificial Sequence
  marker ma0001jh28@36
ccaccgccattgccatcactgccggcttcctccaarcccccgagctcgaacccctcgagtggctccttccc

SEQ ID NO: 39 - DNA, 71 bp, Artificial Sequence
  marker ma0004tn40@36
gacaaaatcgatttctgttaagattcttggacccawaggcctcacgaacaagtctccacccgagcccgaaa

SEQ ID NO: 40 - DNA, 43 bp, Artificial Sequence
  marker ma0004tn32@36
agaaggattcagtgattcacagatcacacacaagakagctcag

SEQ ID NO: 41 - DNA, 52 bp, Artificial Sequence
  marker ma0016fq88@36
tgccaaagacagtaagagcgaagtttgatagtcgayaccgccaatataatga

SEQ ID NO: 42 - DNA, 48 bp, Artificial Sequence
  marker ma0004tn60@34
atcatggcctcattccacaaatcaacacgatctycaaaaattgcaagc

SEQ ID NO: 43 - DNA, 56 bp, Artificial Sequence
  marker ma0016fq93@36
taaagaaagtcaagcccatcaacattgataagtacratggcaagaaggatctaaat

SEQ ID NO: 44 - DNA, 52 bp, Artificial Sequence
  marker ma0016fq89@36
ccaacattagggcgatttgtacgatgtcctccatargcatgatatttgcaag

SEQ ID NO: 45 - DNA, 71 bp, Artificial Sequence
  marker ma0016fq94@36
ttgggcaactccatgacacacaaacttgagtctgayttctataaagttgtaggacacaaagtctagaacac

SEQ ID NO: 46 - DNA, 59 bp, Artificial Sequence
  marker ma0004tn62@36
caagccaataatgtaagggtagataatagggacaasgatgtatccaagaccgacttcat

SEQ ID NO: 47 - DNA, 62 bp, Artificial Sequence
  marker ma0004tn31@27
tctagcaaactccatagtcgcaacatrgaaaccacggtggatcgagcatctacagctctttg

SEQ ID NO: 48 - DNA, 71 bp, Artificial Sequence
  marker ma0016fq50@36
gagcatcatgtttggtaccagctctttccctgacarctcccttctggatgctgatgctgtgttcagtttcg

SEQ ID NO: 49 - DNA, 41 bp, Artificial Sequence
  marker ma0004tn30@36
tttggagctgttgcttcgacgatgattgagagagaycagtg

SEQ ID NO: 50 - DNA, 71 bp, Artificial Sequence
  marker ma0000xk01@36
gtgatcaagcctgagttaaacaagtgcaaacaaacmtactctataggtagcattgtgtcagttgacacttt

SEQ ID NO: 51 - DNA, 71 bp, Artificial Sequence
  marker ma0011cv03@36
tctgcatacgttggcagtttagttctacggggtcgsatgatgctcaggtttggtgaatggggtctctcctt

SEQ ID NO: 52 - DNA, 71 bp, Artificial Sequence
  marker ma0016fq79@36
tgtatctctggatgctgattcaactgtaaatatgasacaatgccagtaattgcatcagtttatatagatag

SEQ ID NO: 53 - DNA, 71 bp, Artificial Sequence
  marker ma0004tn51@36
cctccctcccggcaaccacaacattgaagacagccmctctgtttcacaaattctgactattatcttgagaa

SEQ ID NO: 54 - DNA, 49 bp, Artificial Sequence
  marker ma0004tn53@14
gctatttcagatascgaatgattgtcccctatgatttcattaacaactc

SEQ ID NO: 55 - DNA, 71 bp, Artificial Sequence
  marker ma0000cm74@36
accgacccagtagctgtgtccagtcttgctttaaastttgaagctctcttcttattaaagcagtttttgct

SEQ ID NO: 56 - DNA, 71 bp, Artificial Sequence
  marker ma0000jt04@36
tcagaagtgatcttcttcctttcttctcctaaaatytgtatgagctttctattttcttcctctaattcaga

SEQ ID NO: 57 - DNA, 58 bp, Artificial Sequence
  marker ma0016fq81@35
ccgcgaagaacaaacacgtcacacatgaagcgtcygcttcaaaggatctgagtggatg

SEQ ID NO: 58 - DNA, 71 bp, Artificial Sequence
  marker ma0016fq76@36
ggcaccgaatcgttgatattcgcgtatactgttgcsatgcaggagaccctgaggaaggcggacgagatctt

SEQ ID NO: 59 - DNA, 48 bp, Artificial Sequence
  marker ma0016fq97@18
aaaaaaacgacgtggacygtactaagacgtgtgccagtataaggctgt

SEQ ID NO: 60 - DNA, 46 bp, Artificial Sequence
  marker ma0004tn72@11
ttataggcccyaaaaccccttatagtcgttggagatgcactccaaa

SEQ ID NO: 61 - DNA, 62 bp, Artificial Sequence
  marker ma0004tn96@36
tgtttacgaactatttctggaggctgccacataagmtgggtccaccggttcctacaatttgg

SEQ ID NO: 62 - DNA, 62 bp, Artificial Sequence
  marker ma0016fr46@27
tgggtccaccggttcctacaatttggytatgtgcattaccggatgagtcgtttaaaataatt

SEQ ID NO: 63 - DNA, 71 bp, Artificial Sequence
  marker ma0011rt36@36
gagaaacttctcactgcaaaatcattagaagcagaytgcattcgaactcgcaacacctctctgagaagact

SEQ ID NO: 64 - DNA, 71 bp, Artificial Sequence
  marker ma0022vg84@36
atatccagagaaccgagttactgaattctgcaaagkctaggcagatggaaatagattcatatttgagggaa

SEQ ID NO: 65 - DNA, 47 bp, Artificial Sequence
  marker ma0001hr21@12
actatagacgcrcttaatgagcaacgcgagaagttacaagagcaaag

SEQ ID NO: 66 - DNA, 66 bp, Artificial Sequence
  marker ma0012wu17@36
tgagttagaagagctgaagattgaatctgagaacargcaactgtctttgagacagtgtgggaaatc

SEQ ID NO: 67 - DNA, 66 bp, Artificial Sequence
  marker ma0001hr20@31
gcaactgtctttgagacagtgtgggaaatcraagcatgctggtgtggaaaacctggaagacaacgg

SEQ ID NO: 68 - DNA, 71 bp, Artificial Sequence
  marker ma0000sa77@36
gagcatgggcactttacacttgcacaaggttgaccrtagattttgaatggatagcccacaacgaacccagc

SEQ ID NO: 69 - skipped

SEQ ID NO: 70 - DNA, 201 bp, Artificial Sequence
  marker ma61758s03@101
gatcagcatttttaactgattggttgcagctctatagttgttgctttagtttatgtttaattggaatgaaagatgtaatcaccaatgccaactttgtttaytgggataggtttcgatgtattccatagctgggttcgttgtgggctatccattcaaaatctayggtcaaccttgtgcaagtgtaaagtgcccatgctcatg

SEQ ID NO: 71 - DNA, 58 bp, Artificial Sequence
  marker ma0000qw65@23
tgtgacagcaacagaggaaattrttacaaaactccagagaagtacacaagggataaga

SEQ ID NO: 72 - DNA, 201 bp, Artificial Sequence
  marker ma61758s02@101
tatgtgtagtagattagaaatagagatgcaggtactcagaaaacactgctagacatttaacagtacaagataarcatggctactgatccatctgtgtattygttttttctcatgtataacatagatttggtaaactgtgcgagtctgttacacccgaccttcatccgtttyccctttgctcaatcaggctgggtcaagcag

SEQ ID NO: 73 - DNA, 62 bp, Artificial Sequence
  marker ma0016ft38@27
aatacacagatggatcagtagccatgyttatcttgtactgttaaatgtctagcagtgttttc

SEQ ID NO: 74 - DNA, 71 bp, Artificial Sequence
  marker ma0016ft37@36
tatttctaatctactacacatagggatgagtacacktgtggtccttgaatcaaatatatatgtaaagaaat

SEQ ID NO: 75 - DNA, 61 bp, Artificial Sequence
  marker ma0000wv70@36
tgtggtccttgaatcaaatatatatgtaaagaaatsaaattccacagaatattggagtctt

SEQ ID NO: 76 - DNA, 62 bp, Artificial Sequence
  marker ma0004tp85@27
catttacccaaccaatatgataatttrtacttttcatttttattattcatgcagttttaaac

SEQ ID NO: 77 - DNA, 62 bp, Artificial Sequence
  marker ma0012sk12@36
gaagatcataataggatagcaccacatctaccaccygacaattcacaccatttctgtcgctt

SEQ ID NO: 78 - DNA, 62 bp, Artificial Sequence
  marker ma0022mm17@27
gacaattcacaccatttctgtcgcttyaatatgcagaatacaaataaaacacctcgatccaa

SEQ ID NO: 79 - DNA, 68 bp, Artificial Sequence
  marker ma0004tp78@33
gactgtcttgtagtatttgttctctagttggcytttgtggcgtgctagattcatcagactaatttttg

SEQ ID NO: 80 - DNA, 71 bp, Artificial Sequence
  marker ma0022mm15@36
gtgaccctttaaaccacatttaagtcaagtcattgrccctcttatcattttcttccccttaccaccgatgg

SEQ ID NO: 81 - DNA, 71 bp, Artificial Sequence
  marker ma0004tp79@36
gtcatcattggaacctttcttcttatggccaccctygagacatcccttgatccttaattttctttgccttc

SEQ ID NO: 82 - DNA, 71 bp, Artificial Sequence
  marker ma0004tp82@36
tgtctgttaatggctcaatctctctcccataagctracttgaatttgtccagtgagtaacactcatgcaca

SEQ ID NO: 83 - DNA, 71 bp, Artificial Sequence
  marker ma0016ft40@36
caagtatcccaaacctctagtatttgtcctcagctraaccatatgcaagttagaatgatactcaatattat

SEQ ID NO: 84 - DNA, 68 bp, Artificial Sequence
  marker ma0016ft28@36
gaaaaaatatttcataagtaagcatgtcatgtcagytagatcgggtggatattatgaattgtgaatat

SEQ ID NO: 85 - DNA, 53 bp, Artificial Sequence
  marker ma0004tq11@36
ggttcagagattttttcaacaaaaaagacctacagrcacctaatgggccatac

SEQ ID NO: 86 - DNA, 69 bp, Artificial Sequence
  marker ma0004tq10@34
tcttgatagtcctgttatgtactgcacatcaaaygataaaactcgtctcatatgaattatttagtatta

SEQ ID NO: 87 - DNA, 68 bp, Artificial Sequence
  marker ma0012aq25@36
attttatcattgaattactaggactgtaccatactragtggagatgtcatcataacaaatcttgcaca

SEQ ID NO: 88 - DNA, 201 bp, Artificial Sequence
  marker ma61757s01@101
atgtttttgatacttaaggcatccaatttgtagctactgctctagttctaggatttggaraactccttgtcgaattgtcccacagagctaatgattgtgckaacaaaatgacatgatgctgattgttctacgcctcttgcttaataccctgtgaagtttaattatttggtggtacacttgaccttggaagtcatgacctcc

SEQ ID NO: 89 - DNA, 58 bp, Artificial Sequence
  marker ma0016ft65@23
ctttgtttttacgaaacccgctytagttatactgaaggaactgatcatttctttatct

SEQ ID NO: 90 - DNA, 71 bp, Artificial Sequence
  marker ma0004tq05@36
aacagttgatcatggcatgcaaaagaataacatacrctattgtttttaacataggttgggagatacaagag

SEQ ID NO: 91 - DNA, 40 bp, Artificial Sequence
  marker ma0004tq06@5
gtaakagatataaaccatcatttgcaatctaatgtaccat

SEQ ID NO: 92 - DNA, 201 bp, Artificial Sequence
  marker ma61757s03@101
gytctgtgacttgcagaatgtactatcattattgcaaatgccacatcccacagaccttagatacaagtgacggaaagttctgtgtttactactatatattytactttctccaatatattttggaacaagctgtacagctctttattgtattgcgttgagtcraaataagtgatggggttattttttttgtttttttattac

SEQ ID NO: 93 - DNA, 201 bp, Artificial Sequence
  marker ma61757s04@101
cctgcatgaatcttgtcatattctacaagcgctaacatagygccatcctaatccatgtgcaacatgaaatatttgtttatgctgcatttggactatcatgygttgcaagattcataaatcataatgccaacaagttgtcctggtattatgactatgttagtacattttagatggattcatacttcgctcttgtcctttgca

SEQ ID NO: 94 - DNA, 61 bp, Artificial Sequence
  marker ma0004tq13@26
cccttagttctttgggtgcacatccrcttttgttccagacttccagtcaactaggtgtcca

SEQ ID NO: 95 - DNA, 58 bp, Artificial Sequence
  marker ma0016ft76@23
tccttttgctctgtctgccacarttttcattcagttaaaagaatatatttgagcaaac

SEQ ID NO: 96 - DNA, 40 bp, Artificial Sequence
  marker ma0016ft75@5
gggartaaaattctctcattgtcgttgaaatttatacaga

SEQ ID NO: 97 - DNA, 60 bp, Artificial Sequence
  marker ma0022xj97@36
ttactttgattgccactattctaagtttgcattttstttgttgcaattcatgttaaaatc

SEQ ID NO: 98 - DNA, 41 bp, Artificial Sequence
  marker ma0004tq07@6
tgtcaygactgaaggtgactgtatctgcttaaagagtatta

SEQ ID NO: 99 - DNA, 56 bp, Artificial Sequence
  marker ma0016ft73@31
gtgtaatagacaataagtgcacattagaacractctttaatattcctaactacttc

SEQ ID NO: 100 - DNA, 71 bp, Artificial Sequence
  marker ma0004tq16@36
gtatggtccgacacatgtgcaaaaagtgctagagakcttctataggccatcggtctgatgagtatcgttgg

SEQ ID NO: 101 - DNA, 61 bp, Artificial Sequence
  marker ma0016ft77@36
ggccatcggtctgatgagtatcgttggatggtacgrtggaactatgtagttggcatggtgc

SEQ ID NO: 102 - DNA, 43 bp, Artificial Sequence
  marker ma0016ft86@8
tccttggragccgttagtttcacagaattacacatcatatatt

SEQ ID NO: 103 - DNA, 71 bp, Artificial Sequence
  marker ma0010yr95@36
gaaatcaatatcacacatcttttaaaacttatctargtgttaacacttcttatcgatgtagcatttgcacc

SEQ ID NO: 104 - DNA, 43 bp, Artificial Sequence
  marker ma0011dy20@8
gatgtctrgtttattctcattcatcaacatatatggggtctta

SEQ ID NO: 105 - DNA, 201 bp, Artificial Sequence
  marker ma61755s04@101
tttaacgaaaaaaaggaaaagaatgaccccagcttccatattttctgactatccatctycaccctttggatttctaaccttagatgaaaggaagttgggamtggcgttctggcttgagtttatttacatcctttaattctacaactccaaacgaaaagaagtttggccaattttcctatgtccaatgagttcccgggcagc

SEQ ID NO: 106 - skipped

SEQ ID NO: 107 - DNA, 201 bp, Artificial Sequence
  marker ma61755s03@101
actgtcttgttgcttttatagtgcaattttaygtcttgattcctgcctatttttttccttttcttttttgcttctcgatacattcacctactgtcatttgkccatgttttttgctaggtcagaaattgcttggaaaaatgcaaattatgcacaaattctagaatatcttcgggaggaatatgaactgaatcagcgttttgg

SEQ ID NO: 108 - DNA, 201 bp, Artificial Sequence
  marker ma61755s02@101
aaattcatatcaaaatgaacaaatctcagtcagctttgraccagttgcacatactttgtaacattgacctatgctacgaattgatttccttgtgattttgrttgatatttatgcagaacattggagtgaaggataacagattttgctatcgctatgtggttgttttccagtatggttctgctgtgcttttcaacattgctg

SEQ ID NO: 109 - DNA, 201 bp, Artificial Sequence
  marker ma61755s01@101
ccgccccaggaggaggaggacgagggcccagcacgctacatccccgtcaaagcctacttcctctccaccaggtaagcacgcagcgccgctcccgcgcttgytcgataggtgtttgatgatttatcatcgaggaaacattcgggactttcgggtgctaccytactattctaaaaatgtccatgccaattggcactgcgaact

SEQ ID NO: 110 - DNA, 67 bp, Artificial Sequence
  marker ma0016ft81@36
cactaaggtacgaggaaccatacctcatatttccartgcatcaattattactgctttccatcaaggg

SEQ ID NO: 111 - DNA, 50 bp, Artificial Sequence
  marker ma0016ft78@36
tttttgctctagctgacaaatgtgtcagtttggccragggttgagcatgg

SEQ ID NO: 112 - DNA, 45 bp, Artificial Sequence
  marker ma0016ft83@36
gtctacttttgcgcaagccatgaataagaccttcgrcaatttgat

SEQ ID NO: 113 - DNA, 45 bp, Artificial Sequence
  marker ma0004tq35@10
gcgtcacacrcacgaggctgaatcctgaaaaggtcatctttggtg

SEQ ID NO: 114 - DNA, 51 bp, Artificial Sequence
  marker ma0004tq36@16
accactattttcagtyccatagagtaatggtagtgacttcttatccgctga

SEQ ID NO: 115 - DNA, 62 bp, Artificial Sequence
  marker ma0004tq32@36
ccaacaacatggtctagacgacaaagtcgctcagcrgaagcatccaacacgggcagagcatc

SEQ ID NO: 116 - DNA, 40 bp, Artificial Sequence
  marker ma0022xj98@10
cgcttttgcyaaggtctcgctcaatgggtccattggcaaa

SEQ ID NO: 117 - DNA, 68 bp, Artificial Sequence
  marker ma0004tq24@33
ccggtagaagcaagagaggagtcagacgagggygtgatcccgacaaacataaattgggtcatccacac

SEQ ID NO: 118 - DNA, 50 bp, Artificial Sequence
  marker ma0004tq26@36
tggagctgctgctagcaagcctctttcccttgtccraagggctagggcca

SEQ ID NO: 119 - DNA, 71 bp, Artificial Sequence
  marker ma0004tq30@36
ctctataatcctaatccatcacatatcaactatgcytgtatctaataggataatctaatattttgccatct

SEQ ID NO: 120 - DNA, 52 bp, Artificial Sequence
  marker ma0016ft97@17
gatctgccactgcttcrcttagactataaaaggagagcacatacgatgcagg

SEQ ID NO: 121 - DNA, 41 bp, Artificial Sequence
  marker ma0004tq33@6
ctacaktacaagtcatcgattatttatcattcaacatttat

SEQ ID NO: 122 - DNA, 67 bp, Artificial Sequence
  marker ma0016ft92@32
ctcagttcagtttattgcgcggcagcttttcstgattgagctgagctggaaagtgctgaacccagta

SEQ ID NO: 123 - DNA, 43 bp, Artificial Sequence
  marker ma0004tq39@8
tatcctcygtttatatacaaacatagtacaacgcctatgcgtt

SEQ ID NO: 124 - DNA, 67 bp, Artificial Sequence
  marker ma0022mm26@36
tcgagcgatgcaatgcatgtggcttcctatcgtatyaggattgggtgctttctcaaggcccatctgc

SEQ ID NO: 125 - DNA, 46 bp, Artificial Sequence
  marker ma0016fu15@11
aaaggctaaasaaaagatctgatatactgcaaaacaaaacttgact

SEQ ID NO: 126 - DNA, 44 bp, Artificial Sequence
  marker ma0010yr98@9
actcataayctcacgctctcaacattgatggtgaacagacaata

SEQ ID NO: 127 - DNA, 65 bp, Artificial Sequence
  marker ma0022mm25@36
aacattgatggtgaacagacaatattatggcgagayaaaggctaaagaaaatatctgatatactg

SEQ ID NO: 128 - DNA, 40 bp, Artificial Sequence
  marker ma0016ft98@36
tttatttatttatttacatagatcgcgcagcatccrccct

SEQ ID NO: 129 - DNA, 40 bp, Artificial Sequence
  marker ma0004tq38@5
ccctycaatgttctacactgcctgctggccacaaatctgc

SEQ ID NO: 130 - DNA, 46 bp, Artificial Sequence
  marker ma0004tq54@36
ctatgcgctacagtacaagagttgtacgtaagaatycgctagattc

SEQ ID NO: 131 - DNA, 71 bp, Artificial Sequence
  marker ma0016fu20@36
aagattttgttatggcaaaacctgcccagggcgatytcatgggagtaaactcagcccattacaagtttcat

SEQ ID NO: 132 - DNA, 71 bp, Artificial Sequence
  marker ma0016fu17@36
atccacgggaaatatggcatgttatattcgcatatrgtctgtagcataggtaaatatggaaagaaaggata

SEQ ID NO: 133 - DNA, 59 bp, Artificial Sequence
  marker ma0004tq57@36
atttgtttttccttgacacaacttccaagattgttycacaggttgccttcaattccctc

SEQ ID NO: 134 - DNA, 71 bp, Artificial Sequence
  marker ma0016fu11@36
tcaacactaacacgtatgagatgacttcccaacccwtagaatctagttatctttcctctaatcgtcttgtt

SEQ ID NO: 135 - DNA, 60 bp, Artificial Sequence
  marker ma0016fu10@36
tattacttagataatcattagcatttttaagactakaaatggaacaatgccatgaagaca

SEQ ID NO: 136 - DNA, 40 bp, Artificial Sequence
  marker ma0016fu09@5
ttcarcacataataaatcatttttcatcttttctaaagca

SEQ ID NO: 137 - DNA, 42 bp, Artificial Sequence
  marker ma0004tq43@36
tggcctcagacgaatcgaaactccatctcccatctraggttg

SEQ ID NO: 138 - DNA, 58 bp, Artificial Sequence
  marker ma0016fu01@36
taaggacttcaccaatgacttgagcattagtcgcaygacgtcgtgatccgtccatctt

SEQ ID NO: 139 - DNA, 64 bp, Artificial Sequence
  marker ma0004tq42@36
tcgatcaccttgctctttcctttagcgtcattgctrgcgatctcttcaggttgttagacacaac

SEQ ID NO: 140 - DNA, 61 bp, Artificial Sequence
  marker ma0016fu05@29
gcgatctcttcaggttgttagacacaacrgaagagaccgaatctaatactaattgagagca

SEQ ID NO: 141 - DNA, 55 bp, Artificial Sequence
  marker ma0016ft99@36
gaagaaatccgtagcatcgtcaccctatgtgtaatyggcggccgcacaggacagg

SEQ ID NO: 142 - DNA, 47 bp, Artificial Sequence
  marker ma0016fu07@12
ggtatggccaawttttggcttggtacaagatgccctcagctgaaagc

SEQ ID NO: 143 - DNA, 45 bp, Artificial Sequence
  marker ma0010yt02@36
gtgattgggagggcatagatattcagtcaaccagakaatttcttt

SEQ ID NO: 144 - DNA, 71 bp, Artificial Sequence
  marker ma0000gu42@36
tcatccagttgtttggcttccccaccagggtctaargtccttaacttttcagatagttgtttgattttgcg

SEQ ID NO: 145 - DNA, 101 bp, Artificial Sequence
  marker ma52981s02@51
gttctttttctcccttttgtactttggggtttgggcctctctttctcttcmcctgcgagcctctttcctcgctcttgtctcctgggcgagttctgaatctg

SEQ ID NO: 146 - DNA, 101 bp, Artificial Sequence
  marker ma52981s01@51
gttccacacacctccagtgacctgacaaccacaccgcaataatcgtattcmgttctttttctcccttttgtactttggggtttgggcctctctttctcttc

SEQ ID NO: 147 - DNA, 68 bp, Artificial Sequence
  marker ma0004tq83@33
taaaagaaagtcgcattggcagtgacggctacycgcaacttctcatgtccctcgcaacgtaaatgcat

SEQ ID NO: 148 - DNA, 67 bp, Artificial Sequence
  marker ma0004tq66@36
ctagtgatgcaaaagcatctacactaccatacgcargcaggactttcccaccatgtgatctaaaagt

SEQ ID NO: 149 - DNA, 44 bp, Artificial Sequence
  marker ma0016fu32@9
tcaaggctwtgtgtttgtccaattcaaaagtggagagtccgttt

SEQ ID NO: 150 - DNA, 47 bp, Artificial Sequence
  marker ma0011zk10@36
ggaatttttgagaaaacgagtcttcaatggaacgcraggtcaaacga

SEQ ID NO: 151 - DNA, 71 bp, Artificial Sequence
  marker ma0004tq62@36
aaggcttccggaacgggagctgaagaaaagagaacmggtgtctgggcgccggagcgtttgagcgctgaaaa

SEQ ID NO: 152 - DNA, 40 bp, Artificial Sequence
  marker ma0004tq68@36
cttggcttcttgcgtctagctactgagcttcttctrcctc

SEQ ID NO: 153 - DNA, 71 bp, Artificial Sequence
  marker ma0004tq78@36
cacaatcacaaactctgagcatctagcagtcaaacrcatgaagacagagcagtttacaactttacatccat

SEQ ID NO: 154 - DNA, 71 bp, Artificial Sequence
  marker ma0004tq59@36
tgcgcggatccagttggtctccttcgcacacaaatytcccacatttgcgcccattttccgtgcatgcagct

SEQ ID NO: 155 - DNA, 40 bp, Artificial Sequence
  marker ma0022mm29@9
cacttgtgscttaaacagcaccaaattgtgcctaggcaat

SEQ ID NO: 156 - DNA, 101 bp, Artificial Sequence
  marker ma53009s01@51
gttttgggacccaaccactacctccacattttgctgcaaagttcaaccacrcacgctctctatgccgattctttaacatggagccgttaatgtagttttta

SEQ ID NO: 157 - DNA, 40 bp, Artificial Sequence
  marker ma0016fu41@7
cacgctstctatgccgattctttaacatggagccgttaat

SEQ ID NO: 158 - DNA, 54 bp, Artificial Sequence
  marker ma0016fu35@19
gtttgttttccattgttcytaggccattgttcttatgccatgttggccatattt

SEQ ID NO: 159 - DNA, 58 bp, Artificial Sequence
  marker ma0016fu36@36
ttcatgaccatgaagtctctaccaaaggagtgtgaracatttcatgttcagtacaaca

SEQ ID NO: 160 - DNA, 71 bp, Artificial Sequence
  marker ma0004tq69@36
actcatgtgcaaatattcatgttgtcaactctctayagggactccgttcaagccaaaggctcctaaaggtc

SEQ ID NO: 161 - DNA, 62 bp, Artificial Sequence
  marker ma0016fu28@36
tgtagagactagaagagttgtgatccatggcaacargccctgtgattttcccaataggcacg

SEQ ID NO: 162 - DNA, 71 bp, Artificial Sequence
  marker ma0016fu26@36
gttcctcctcctaccactaaagctcctactatcaaygctacttgtccaccaccattcacttatccatatcc

SEQ ID NO: 163 - DNA, 71 bp, Artificial Sequence
  marker ma0010yt03@36
ggccgatgtgtgaggtgccctatggtgcgaaagggrcctagggttgtattgttgagagaaatatcctcatc

SEQ ID NO: 164 - DNA, 40 bp, Artificial Sequence
  marker ma0016fu45@36
aagtgggacatttactacactgttctgcatcacacyggac

SEQ ID NO: 165 - DNA, 71 bp, Artificial Sequence
  marker ma0022mm28@36
gcactgaactgccgccacgtcaccaaagccgttggrgcgaagtcaatcgttgaacctatcagacggtctgg

SEQ ID NO: 166 - DNA, 53 bp, Artificial Sequence
  marker ma0022xj99@18
aatcatgattgtccccgyaaaaaatggtgcagtgttttccttgtattatagta

SEQ ID NO: 167 - DNA, 65 bp, Artificial Sequence
  marker ma0016fu43@30
ggatagagtatttagcaaaagaataatgtstccatatacttaataaatgatacttatgtcataca

SEQ ID NO: 168 - DNA, 71 bp, Artificial Sequence
  marker ma0004tq81@36
agtcaggcacacagcacatctggtctctgcaaagamcaaagcaagcgcgcgcgtatacgcatgctggagag

SEQ ID NO: 169 - DNA, 57 bp, Artificial Sequence
  marker ma0016fu29@22
tgcatcaccatcattccgtgcrtgatcgtccctttaattacttttggttaagttaac

SEQ ID NO: 170 - DNA, 44 bp, Artificial Sequence
  marker ma0022mm31@36
atagtgacctctagctaagaactgatactaaactarttttgaca

SEQ ID NO: 171 - DNA, 40 bp, Artificial Sequence
  marker ma0010yt04@9
tttaggttrcagagagaatttctcggagcattgaacttgt

SEQ ID NO: 172 - DNA, 67 bp, Artificial Sequence
  marker ma0004tr07@32
cagagagaatttctcggagcattgaacttgtygtctacggcccatttcacgtaattcctgataaagt

SEQ ID NO: 173 - DNA, 59 bp, Artificial Sequence
  marker ma0012fz92@36
agtatataatatgccaatctgggatcggacgagtaygatgcagccacgttttccagtcg

SEQ ID NO: 174 - DNA, 71 bp, Artificial Sequence
  marker ma0001ac17@36
cgctacgcgtggaagccgaaccttggtcagaagatmaacgaaaccgtaagctctcctcccatcgccttcgc

SEQ ID NO: 175 - DNA, 71 bp, Artificial Sequence
  marker ma0012pg35@36
aggttccgacctctgtcagaaatcaccggcgtctgycagcgctgccgggatcaaatcgactggaaacgcag

SEQ ID NO: 176 - DNA, 71 bp, Artificial Sequence
  marker ma0000en22@36
gcgctgccgggatcaaatcgactggaaacgcaggtsgggaattgtcatccctcatcggttgtctaagctca

SEQ ID NO: 177 - DNA, 40 bp, Artificial Sequence
  marker ma0000en21@36
atttagctagtgtgtagcttcgattgtctcctaggyccaa

SEQ ID NO: 178 - DNA, 50 bp, Artificial Sequence
  marker ma0016fu62@36
aatctctttccctgtttagcacgtatactgagcagygagagaggtgacgc

SEQ ID NO: 179 - DNA, 71 bp, Artificial Sequence
  marker ma0004tq90@36
atgcagaggtgaattttggccactcaatctcttgtktcactaggcctggttgttacttgttaaacattgat

SEQ ID NO: 180 - DNA, 101 bp, Artificial Sequence
  marker ma08364s01@51
tctcttgtgtcactaggcctggttgttacttgttaaacattgataggttgrattgttactgatccgggtttgctgctgcttaatgcagattgctccaagga

SEQ ID NO: 181 - DNA, 71 bp, Artificial Sequence
  marker ma0011zt60@36
ctcggcatatgtgctaagtgctgcacatgtgttaaygagcttgtcggaaggtgtgattcctgctgtgcatt

SEQ ID NO: 182 - DNA, 42 bp, Artificial Sequence
  marker ma0004tq98@7
taattckatttagggatgcgaatgaggaggaaagtgaacgca

SEQ ID NO: 183 - DNA, 71 bp, Artificial Sequence
  marker ma0016fu52@36
ggaacgacgtacacttcttcgtattgtgagtactgyttttcaacttttcacactgaacaaacttttgttcg

SEQ ID NO: 184 - DNA, 46 bp, Artificial Sequence
  marker ma0016fu55@11
tttaaaacttmagtattttctcactgttgccaattgaataattttt

SEQ ID NO: 185 - DNA, 59 bp, Artificial Sequence
  marker ma0011dy21@24
gagctagggtcctgttttggcaargttccatccagtgaaaccttacgatacaatgaaaa

SEQ ID NO: 186 - DNA, 71 bp, Artificial Sequence
  marker ma0004tq88@36
cagatgtagaggctggagattttgtggaagattaartttggaatgctttatgctgtaaagacgatactttc

SEQ ID NO: 187 - DNA, 40 bp, Artificial Sequence
  marker ma0022xk01@5
tggtmggattgtcgcgcatttccttatgtttgttcgaaag

SEQ ID NO: 188 - DNA, 71 bp, Artificial Sequence
  marker ma0016fu57@36
ggattgtcgcgcatttccttatgtttgttcgaaagyttggaattggaagatgttaaactgcgttgatttaa

SEQ ID NO: 189 - DNA, 71 bp, Artificial Sequence
  marker ma0010yt06@36
aacgttgcaaattaccgatatgcaaagcaaagcgtyacgcaggtaaagccagatcttgtccgtaggctgcc

SEQ ID NO: 190 - DNA, 47 bp, Artificial Sequence
  marker ma0001dg29@12
gactcagtacarctgcatgtggtggaggaattatggcaaacattgca

SEQ ID NO: 191 - DNA, 40 bp, Artificial Sequence
  marker ma0016fu59@7
cagtggrcccgacaatgtaaggaactcgtagcaaaccttt

SEQ ID NO: 192 - DNA, 49 bp, Artificial Sequence
  marker ma0016fu58@34
cccgacaatgtaaggaactcgtagcaaacctttyctaaagtgcccttca

SEQ ID NO: 193 - DNA, 42 bp, Artificial Sequence
  marker ma0004tr05@36
ggttgaacctagtgagaggtcaactaataagagatmaaaagg

SEQ ID NO: 194 - DNA, 57 bp, Artificial Sequence
  marker ma0004tr10@22
aacgcttcgactactacaaacstaatgtttataggataggaaattctccgatcacct

SEQ ID NO: 195 - DNA, 41 bp, Artificial Sequence
  marker ma0004tr03@36
gataggaaattctccgatcacctacacaagaatatrcgaat

SEQ ID NO: 196 - DNA, 69 bp, Artificial Sequence
  marker ma0004tq84@36
ggagttgctggatcatataccagaaaccagtatttycagagaagtgcacgagagagcagtgacagtgaa

SEQ ID NO: 197 - DNA, 52 bp, Artificial Sequence
  marker ma0016fu56@17
atgtgttttgtgttccraaaacttcaaatctaaccatgatgaattggatgga

SEQ ID NO: 198 - DNA, 44 bp, Artificial Sequence
  marker ma0011jn34@9
agagaattkttttgagtcacccttgatcccattagcattgtttt

SEQ ID NO: 199 - DNA, 60 bp, Artificial Sequence
  marker ma0016fu80@25
tactactacggttcataccatcatyagcactatatataaacaccgtatcgatcggcccgt

SEQ ID NO: 200 - DNA, 42 bp, Artificial Sequence
  marker ma0004tr23@36
tgttgcatcgtaaacgacctgctgatcggtgtcctsatcggc

SEQ ID NO: 201 - DNA, 1143 bp, Artificial Sequence
  cDNA of Zm00001d043358 (restorer)
atggggcggcttcgcgcgtgctgccgtctccaccgcttcctcgcgccgccgccggcacaagtgccatcaccgggacaatctctggcccgcgggtccaacaccgctaccgctcacgtagcgcctttctcgaggcttctctcgtcggccgcggccgttgcgccccacgacacacgcgactcgggcctcggcggttcggcgtactgggcctggattcgcgcggcgaccgagtcggccccggctccttcgccgccccaggaggaggaggacgagggcccagcacgctacatccccgtcaaagcctacttcctctccaccagcattgatctgaagagcatgcaagcggagcacgggaatgatattgtacctccgtcgacccgtttgcttaactacattgcgcttcggtactccgagttcccacctgagattatgaacattggagtgaaggataacagattttgctatcgctatgtggttgttttccagtatggttctgctgtgcttttcaacattgctgatcatgaagccgagtactatcttgatataattaggaagcatgcttcaggatggcttccagagatgagaaaagatgattatgcagtggttgagaaaccatccttgacaacatggatgaaaggaggacttgattatatagttcttaaaagtttagatacagacgggattcgcataatttcaagcgttcttggtcaaagtatcgcccttgatcactatattcggcaggtactgaaactcaaagttaatgaacttatcactgaaattaatcgtgtcatggagaaaaccggtaactttaccatgcaaagaaaaaaactctttcaacttgtgggtaaggccaattctaatcttgcggatgttatcatcagacttggtctttttgacaggtcagaaattgcttggaaaaatgcaaattatgcacaaattctagaatatcttcgggaggaatatgaactgaatcagcgttttggaagccttgacttcaaactgaaatttgtggagcataacattcactttcttcaagaggtcctccaaaacagacggtctgatctgctggaatggggtgtcataatactgctgattattgagattgctatctcattatatgaaattgccaaggactccatgatcgat

SEQ ID NO: 202 - protein, 381 aa, Zea mays
MGRLRACCRLHRFLAPPPAQVPSPGQSLARGSNTATAHVAPFSRLLSSAAAVAPHDTRDSGLGGSAYWAWIRAATESAPAPSPPQEEEDEGPARYIPVKAYFLSTSIDLKSMQAEHGNDIVPPSTRLLNYIALRYSEFPPEIMNIGVKDNRFCYRYVVVFQYGSAVLFNIADHEAEYYLDIIRKHASGWLPEMRKDDYAVVEKPSLTTWMKGGLDYIVLKSLDTDGIRIISSVLGQSIALDHYIRQVLKLKVNELITEINRVMEKTGNFTMQRKKLFQLVGKANSNLADVIIRLGLFDRSEIAWKNANYAQILEYLREEYELNQRFGSLDFKLKFVEHNIHFLQEVLQNRRSDLLEWGVIILLIIEIAISLYEIAKDSMID

SEQ ID NO: 203 - DNA, 525 bp, Artificial Sequence
  cDNA of Zm00001d043352 (restorer)
atgaggctgctgcaagcggaggagctatttcggaaggttctggaggtcgggtcgaagaataaggcggctcggctgctggggctcgacgtcggcagcaagtacgtcggggtggccatctccgatgagaagaacagggtcgctatgcctctgagtgttttgtgtcggacaaaaacaaacatcaacttgatggcagatgatttcaaaacattggtttcgatgtattccatagctgggttcgttgtgggctatccattcaaaatctacggtcaaccttgtgcaagtgcaatccaagtaaggcatcttgctggggaactttgtaaaacagggaaacttgatgatctgccctacgcatattgggatgaaaatttcacctcaaagtgtgtagaagccctcttgcatcccctgaatctaaaagacttggacgatgccaaaacaatgacagacaaatttgctgcagtctgtatactccagggttatcttgacaacatgaacagaaaactgagatccacagataagtctgaagca

SEQ ID NO: 204 - protein, 175 aa, Zea mays
MRLLQAEELFRKVLEVGSKNKAARLLGLDVGSKYVGVAISDEKNRVAMPLSVLCRTKTNINLMADDFKTLVSMYSIAGFVVGYPFKIYGQPCASAIQVRHLAGELCKTGKLDDLPYAYWDENFTSKCVEALLHPLNLKDLDDAKTMTDKFAAVCILQGYLDNMNRKLRSTDKSEA

SEQ ID NO: 205 - DNA, 936 bp, Artificial Sequence
  cDNA of Zm00001d043356 (restorer)
atggccttcaagctggccaccaaggccgcggcggcgtcgcccgctgctgctcaccgcgggggtctcgcccgggggccggagggtacgagccgcgttgccttcggaccagcgcctagaaacaaggggctccgcgcggccaacaactccgcgacgcccgtggctaaggaagagagggttgatcgaagtgaaatattgacattggatagcattagacaagttttgattagactagaagacagcatcatattcggccttttggagagagcacagttttgttacaacgctgatacatatgatagcaatgctttccacatggatggttttggcggctctttggttgaatatatggttagagaaactgaaaagctccatgcacaggttgggagatacaagagcccagatgagcaccctttctttcctgaggatctgcctgagccccggttgccacctatgcagtacccaagggttttgcatcccattgccgattctatcaatatcaacaaagagatttggaaaatgtattttgatgaacttcttccaagattggtgaaaaaaggaagtgatggtaatgctggatccagtgctctttgtgacacgacctgcttgcaggcgctctccaaaaggatccactatgggaagtttgtggcagaggctaagtttcaggagtccccggaagcttacatgccagccataatagctcaggaccgtgatcaactcatgcaccttctcacatatgaaacggtggagcgtgctatcgaacacagggtggaagccaaagccaagatcttcgggcaagaggtgaacatcggtgtggaggacaacggcagcccaccggtgtacaagatcgttccgagcttggtcgccgagctgtacagctacagaatcatgccgctaaccaaagaggttcaaatcgcgtatttgcttaggaggctggat

SEQ ID NO: 206 - protein, 312 aa, Zea mays; identical to SEQ ID NO: 12

SEQ ID NO: 207 - DNA, 540 bp, Artificial Sequence
  cDNA of Zm00001d043356 (restorer) - 5′ end
atggctaccgcggcgcgcctcctcctcgtcgtggtgcttctctccgcggcgataccgatcccgtccttcgcgacaagcttcgtcgtcggcgacaagaggcatcgctgggctcccaacgtcaactatacagactgggcggaccgtcaccagttccacgtcggcgactggctcgaattcaggtacgagagggacaggttcgatgtggtgcaggtgaacgagacggcatacgcggcgtgcgacgccagcagccccatcctcagctacagccgcggccacaacttcgtcttccggctcaaccacacgggccggttctacttcatctgcagccgcggctactgctggagtggcatgaaggtctccgtgctcgtccagccgccgccgcccccgtcgctgccgccggcgtcccactcccacgcgtcaagcgccagtgcgcgtgtgcggccggcggccgggctttggtgtgcggctctctctaccttgctgggctgggcggtgctcacgccgccgctgtcgtttcgggtctggagttctggatgtcca

SEQ ID NO: 208 - protein, 180 aa, Zea mays
MATAARLLLVVVLLSAAIPIPSFATSFVVGDKRHRWAPNVNYTDWADRHQFHVGDWLEFRYERDRFDVVQVNETAYAACDASSPILSYSRGHNFVFRLNHTGRFYFICSRGYCWSGMKVSVLVQPPPPPSLPPASHSHASSASARVRPAAGLWCAALSTLLGWAVLTPPLSFRVWSSGCP

(19) United States
(12) Patent Application Publication
(10) Pub. No.: US 2024/0057538 A1
(43) Pub. Date: Feb. 22, 2024

(54) METHODS FOR IDENTIFYING AND SELECTING MAIZE PLANTS WITH CYTOPLASMATIC MALE STERILITY RESTORER GENE

(71) Applicant: KWS SAAT SE & Co. KGaA, Einbeck (DE)

(72) Inventors: Monika KLOIBER-MAITZ, Einbeck (DE); Carsten KNAAK, Göttingen (DE); Hervé DALL'OCCHIO, Caux-et-Sauzens (FR); Jean-Claude CASTELLE, Alzonne (FR)

(73) Assignee: KWS SAAT SE & Co. KGaA, Einbeck (DE)

(21) Appl. No.: 18/268,901

(22) PCT Filed: Dec. 22, 2021
(86) PCT No.: PCT/EP2021/087187

§ 371 Date: Jun. 21, 2023

(30) Foreign Application Priority Data
Dec. 22, 2020 (EP) 20216347.3

(51) Int. Cl. A01H 1/04; A01H 1/02; C12Q 1/6895

(52) U.S. Cl. CPC A01H 1/045; A01H 1/023; C12Q 1/6895; C12Q 2600/13; C12Q 2600/156; C12Q 2600/172

(57) ABSTRACT

The present invention relates to a method for identifying a plant or plant part, such as a maize plant or plant part, comprising a novel restorer of fertility locus, in particular a cytoplasmic male sterility restorer of fertility locus. The invention further relates to molecular markers associated with such locus and the use of such markers in the identification of plants. The invention further relates to methods for generating plants or plant parts comprising the novel restorer of fertility locus.

### FIELD OF THE INVENTION

The invention relates to methods for identifying plants or plant parts, in particular maize plants or plant parts having a restorer of fertility genotype or phenotype, in particular a cytoplasmic fertility restorer of fertility genotype or phenotype. The present invention also relates to plants identified as such, as well as methods for generating such plants. The present invention further relates to polynucleic acids and polypeptides suitable for identifying or generating such plants.

### BACKGROUND OF THE INVENTION

With the aim to inhibit self-pollination of female lines in hybrid production, CMS (cytoplasmic male sterility) is applied in plant breeding programs worldwide. CMS is characterized by maternally inherited mutations, which result in plants that are unable to produce pollen. (Schnable, P. S., & Wise, R. P. (1998). The molecular basis of cytoplasmic male sterility and fertility restoration. Trends in plant science, 3(5), 175-180.)

In order to ensure that the hybrid progeny is entirely fertile and able to produce seeds, male parental lines in hybrid production need to possess so called restorer genes, which cover and cancel the effects of CMS.

For the maize cms plasma cmsC, the major restorer locus RF4 at the beginning of chromosome 8, is well known (WO 2012/047595). Before conversion of a female line to CMSC, it has to be guaranteed, that RF4 is not active in this line. Otherwise, the maintainer locus has to be introgressed before CMS conversion is possible. Defining restorer genotype is done both by marker application and by phenotypic observations.

It is an objective of the present invention to identify new restorer genotypes for use in plant breeding programs, in particular maize plant breeding programs including the use of CMS. The identification of new restorer genotypes, in particular different from the restorer genotypes already known on maize chromosome 8 expands their usability in plant breeding, in particular introgression.

### SUMMARY OF THE INVENTION

The present invention relates to plants or plant parts, in particular maize plants or plants parts having a restorer genotype or phenotype and their use. The restorer genotype or phenotype in particular refers to a cytoplasmic male sterility (CMS) restorer genotype or phenotype, i.e. a genotype or phenotype which restores male fertility.

Preferably, the restorer genotype is caused by one or more restorer genes which are located on maize chromosome 3, i.e. RF-03-01.

The present invention advantageously allows to identify maize lines having a CMS restorer phenotype, which comprise well known restorer genes or loci, such as RF4. The different chromosomal location of the restorer locus of the present invention compared to known restorer loci expands the tool kit for generating as well as maintaining restorer lines, or alternatively for ensuring that an unwanted restorer genotype/phenotype remains absent or can be selected against.

The present invention is in particular captured by any one or any combination of one or more of the below numbered statements 1 to 96, which can be combined with any other statements and/or embodiments.

1. A method for identifying a (maize) plant or plant part, comprising screening for the presence of, detecting, or identifying (a haplotype associated with) a cytoplasmic male sterility (CMS) (fertility) restorer locus on chromosome 3 (RF-03-01).

2. The method according to statement 1, wherein said locus comprises or is comprised in a region on chromosome 3 corresponding to positions 195629901 to 198023573 of B73 AGPv4, or a fragment thereof.

3. The method according to statement 1 or 2, wherein said locus comprises or is comprised in a region on chromosome 3 corresponding to positions 197453646 to 197698278 of B73 AGPv4, or a fragment thereof.

4. The method according to any of statements 1 to 3, wherein said locus comprises one or more of molecular marker(s) (alleles) of Table 4 or Table 5.

5. The method according to any of statements 1 to 4, wherein said locus comprises one or more of a polynucleic acid comprising one or more of SEQ ID NOs: 17 to 200.

6. The method according to any of statements 1 to 5, wherein said locus comprises one or more of a polynucleic acid comprising one or more of SEQ ID NOs: 68 to 140.

7. The method according to any of statements 1 to 6, wherein said locus comprises one or more of Zm00001d043358, Zm00001d043352, Zm00001d043356, and Zm00001d043357.

8. The method according to any of statements 1 to 7, wherein said locus comprises Zm00001d043358.

9. The method according to any of statements 1 to 8, wherein said locus comprises a polynucleic acid comprising one or more of SEQ ID NOs: 1, 5, 9, and 13 or one or more of SEQ ID NOs: 2, 6, 10, and 14, or a polynucleic acid comprising a sequence which is at least 80%, preferably at least 85%, more preferably at least 90%, most preferably at least 95% identical to a sequence as set forth in any one or more of SEQ ID NOs: 1, 5, 9, and 13 or SEQ ID NOs: 2, 6, 10, and 14.

10. The method according to any of statements 1 to 9, wherein said locus comprises a polynucleic acid comprising SEQ ID NOs: 1 or 2, or a polynucleic acid comprising a sequence which is at least 80%, preferably at least 85%, more preferably at least 90%, most preferably at least 95% identical to a sequence as set forth in any one or more of SEQ ID NOs: 1 or 2.

11. The method according to any of statements 1 to 10, comprising screening for the presence of any one or more of SEQ ID NOs: 17 to 200.

12. The method according to any of statements 1 to 11, comprising screening for the presence of any one or more of SEQ ID NOs: 68 to 140.

13. The method according to any of statements 1 to 12, comprising screening for the presence of any one or more of SEQ ID NOs: 70, 72, 76, 79, 86, 88, 92, 93, 99, 104, 105, 107, 108, 109, 115, and 134.

14. The method according to any of statements 1 to 13, comprising screening for the presence of any one or more of SEQ ID NOs: 1, 5, 9, and 13 or one or more of SEQ ID NOs: 2, 6, 10, and 14, or a (unique) fragment thereof, or comprising screening for the presence of a polynucleic acid comprising a sequence which is at least 80%, preferably at least 85%, more preferably at least 90%, most preferably at least 95% identical to a sequence as set forth in any one or more of SEQ ID NOs: 1, 5, 9, and 13 or one or more of SEQ ID NOs: 2, 6, 10, and 14, or a (unique) fragment thereof.

15. The method according to any of statements 1 to 14, comprising screening for the presence of SEQ ID NOs: 1 or 2, or a (unique) fragment thereof, or comprising screening for the presence of a polynucleic acid comprising a sequence which is at least 80%, preferably at least 85%, more preferably at least 90%, most preferably at least 95% identical to a sequence as set forth in SEQ ID NOs: 1 or 2, or a (unique) fragment thereof.

16. The method according to any of statements 1 to 14, comprising screening for the presence of one or more of Zm00001d043358, Zm00001d043352, Zm00001d043356, and/or Zm00001d043357, or a fragment thereof, wherein
Zm00001d043358
   has a genomic sequence of SEQ ID NO: 1 or 2, or a genomic sequence which is at least 80%, preferably at least 85%, more preferably at least 90%, most preferably at least 95% identical to SEQ ID NO: 1 or 2;
   has a coding sequence of SEQ ID NO: 201 or 3, or a coding sequence which is at least 80%, preferably at least 85%, more preferably at least 90%, most preferably at least 95% identical to SEQ ID NO: 201 or 3; and/or
   encodes a protein having a sequence of SEQ ID NO: 202 or 4, or a protein having a sequence which is at least 80%, preferably at least 85%, more preferably at least 90%, most preferably at least 95% identical to SEQ ID NO: 202 or 4;
Zm00001d043352
   has a genomic sequence of SEQ ID NO: 5 or 6, or a genomic sequence which is at least 80%, preferably at least 85%, more preferably at least 90%, most preferably at least 95% identical to SEQ ID NO: 5 or 6;
   has a coding sequence of SEQ ID NO: 203 or 7, or a coding sequence which is at least 80%, preferably at least 85%, more preferably at least 90%, most preferably at least 95% identical to SEQ ID NO: 203 or 7; and/or
   encodes a protein having a sequence of SEQ ID NO: 204 or 8, or a protein having a sequence which is at least 80%, preferably at least 85%, more preferably at least 90%, most preferably at least 95% identical to SEQ ID NO: 204 or 8;
Zm00001d043356
   has a genomic sequence of SEQ ID NO: 9 or 10, or a genomic sequence which is at least 80%, preferably at least 85%, more preferably at least 90%, most preferably at least 95% identical to SEQ ID NO: 9 or 10;
   has a coding sequence of SEQ ID NO: 205 or 11, or a coding sequence which is at least 80%, preferably at least 85%, more preferably at least 90%, most preferably at least 95% identical to SEQ ID NO: 205 or 11; and/or
   encodes a protein having a sequence of SEQ ID NO: 206 or 12, or a protein having a sequence which is at least 80%, preferably at least 85%, more preferably at least 90%, most preferably at least 95% identical to SEQ ID NO: 206 or 12;
Zm00001d043357
   has a genomic sequence of SEQ ID NO: 13 or 14, or a genomic sequence which is at least 80%, preferably at least 85%, more preferably at least 90%, most preferably at least 95% identical to SEQ ID NO: 13 or 14;
   has a coding sequence of SEQ ID NO: 207 or 15, or a coding sequence which is at least 80%, preferably at least 85%, more preferably at least 90%, most preferably at least 95% identical to SEQ ID NO: 207 or 15; and/or
   encodes a protein having a sequence of SEQ ID NO: 208 or 16, or a protein having a sequence which is at least 80%, preferably at least 85%, more preferably at least 90%, most preferably at least 95% identical to SEQ ID NO: 208 or 16.

17. The method according to any of statements 1 to 16, comprising screening for the presence of any one or more molecular markers (alleles) of Table 4 or Table 5.

18. A method for identifying a (maize) plant or plant part (having a restorer of fertility locus on chromosome 3), comprising screening for the presence of any one or more molecular markers (alleles) of Table 4 or Table 5.

19. A method for identifying a (maize) plant or plant part (having a restorer of fertility locus on chromosome 3), comprising screening for the presence of any one or more of SEQ ID NOs: 17 to 200.

20. A method for identifying a (maize) plant or plant part (having a restorer of fertility locus on chromosome 3), comprising screening for the presence of any one or more of SEQ ID NOs: 68 to 140.

21. A method for identifying a (maize) plant or plant part (having a restorer of fertility locus on chromosome 3), comprising screening for the presence of any one or more of SEQ ID NOs: 70, 72, 76, 79, 86, 88, 92, 93, 99, 104, 105, 107, 108, 109, 115, and 134.

22. A method for identifying a (maize) plant or plant part (having a restorer of fertility locus on chromosome 3), comprising screening for the presence of any one or more of SEQ ID NOs: 1, 5, 9, and 13, or a (unique) fragment thereof, or a polynucleic acid comprising a sequence which is at least 80%, preferably at least 85%, more preferably at least 90%, most preferably at least 95% identical to a sequence as set forth in any one or more of SEQ ID NOs: 1, 5, 9 and 13; or one or more of SEQ ID NOs: 2, 6, 10, and 14, or a (unique) fragment thereof, or a polynucleic acid comprising a sequence which is at least 80%, preferably at least 85%, more preferably at least 90%, most preferably at least 95% identical to a sequence as set forth in any one or more of SEQ ID NOs: 2, 6, 10, and 14, or a (unique) fragment thereof.

23. A method for identifying a (maize) plant or plant part (having a restorer of fertility locus on chromosome 3), comprising screening for the presence of SEQ ID NOs: 1 or 2, or a (unique) fragment thereof, or comprising screening for the presence of a polynucleic acid comprising a sequence which is at least 80%, preferably at least 85%, more preferably at least 90%, most preferably at least 95% identical to a sequence as set forth in SEQ ID NO: 1 or 2, or a (unique) fragment thereof.

24. A method for identifying a (maize) plant or plant part (having a restorer of fertility locus on chromosome 3), comprising screening for the presence of Zm00001d043358, Zm00001d043352, Zm00001d043356, and/or Zm00001d043357, or a fragment thereof, wherein
Zm00001d043358
   has a genomic sequence of SEQ ID NO: 1 or 2, or a genomic sequence which is at least 80%, preferably at least 85%, more preferably at least 90%, most preferably at least 95% identical to SEQ ID NO: 1 or 2;
   has a coding sequence of SEQ ID NO: 201 or 3, or a coding sequence which is at least 80%, preferably at least 85%, more preferably at least 90%, most preferably at least 95% identical to SEQ ID NO: 201 or 3; and/or
   encodes a protein having a sequence of SEQ ID NO: 202 or 4, or a protein having a sequence which is at least 80%, preferably at least 85%, more preferably at least 90%, most preferably at least 95% identical to SEQ ID NO: 202 or 4;
Zm00001d043352
   has a genomic sequence of SEQ ID NO: 5 or 6, or a genomic sequence which is at least 80%, preferably at least 85%, more preferably at least 90%, most preferably at least 95% identical to SEQ ID NO: 5 or 6;
   has a coding sequence of SEQ ID NO: 203 or 7, or a coding sequence which is at least 80%, preferably at least 85%, more preferably at least 90%, most preferably at least 95% identical to SEQ ID NO: 203 or 7; and/or
   encodes a protein having a sequence of SEQ ID NO: 204 or 8, or a protein having a sequence which is at least 80%, preferably at least 85%, more preferably at least 90%, most preferably at least 95% identical to SEQ ID NO: 204 or 8;
Zm00001d043356
   has a genomic sequence of SEQ ID NO: 9 or 10, or a genomic sequence which is at least 80%, preferably at least 85%, more preferably at least 90%, most preferably at least 95% identical to SEQ ID NO: 9 or 10;
   has a coding sequence of SEQ ID NO: 205 or 11, or a coding sequence which is at least 80%, preferably at least 85%, more preferably at least 90%, most preferably at least 95% identical to SEQ ID NO: 205 or 11; and/or
   encodes a protein having a sequence of SEQ ID NO: 206 or 12, or a protein having a sequence which is at least 80%, preferably at least 85%, more preferably at least 90%, most preferably at least 95% identical to SEQ ID NO: 206 or 12;
Zm00001d043357
   has a genomic sequence of SEQ ID NO: 13 or 14, or a genomic sequence which is at least 80%, preferably at least 85%, more preferably at least 90%, most preferably at least 95% identical to SEQ ID NO: 13 or 14;
   has a coding sequence of SEQ ID NO: 207 or 15, or a coding sequence which is at least 80%, preferably at least 85%, more preferably at least 90%, most preferably at least 95% identical to SEQ ID NO: 207 or 15; and/or
   encodes a protein having a sequence of SEQ ID NO: 208 or 16, or a protein having a sequence which is at least 80%, preferably at least 85%, more preferably at least 90%, most preferably at least 95% identical to SEQ ID NO: 208 or 16.

25. An (isolated) polynucleic acid comprising one or more molecular marker (allele) of Table 4 or Table 5, or the complement or reverse complement of said polynucleic acid.

26. An (isolated) polynucleic acid comprising one or more nucleotides corresponding to an SNP of Table 4, or the complement or reverse complement of said polynucleic acid.

27. An (isolated) polynucleic acid comprising at least 15 contiguous nucleotides comprised in a region corresponding to a region flanked by any of the indicated 5′ and 3′ positions of the table below and comprising the nucleotide corresponding to the position of the indicated SNP referenced to maize chromosome 3, B73 AGPv4

| 5' (−100 nt) | 5' (−50 nt) | SNP | 3' (+50 nt) | 3' (+100 nt) |
|---|---|---|---|---|
| 195629801 | 195629851 | 195629901 | 195629951 | 195630001 |
| 195639594 | 195639644 | 195639694 | 195639744 | 195639794 |
| 195677699 | 195677749 | 195677799 | 195677849 | 195677899 |
| 195678256 | 195678306 | 195678356 | 195678406 | 195678456 |
| 195680690 | 195680740 | 195680790 | 195680840 | 195680890 |
| 195732836 | 195732886 | 195732936 | 195732986 | 195733036 |
| 195733816 | 195733866 | 195733916 | 195733966 | 195734016 |
| 195783601 | 195783651 | 195783701 | 195783751 | 195783801 |
| 196069986 | 196070036 | 196070086 | 196070136 | 196070186 |
| 196198636 | 196198686 | 196198736 | 196198786 | 196198836 |
| 196244614 | 196244664 | 196244714 | 196244764 | 196244814 |
| 196653646 | 196653696 | 196653746 | 196653796 | 196653846 |
| 196693401 | 196693451 | 196693501 | 196693551 | 196693601 |
| 196702711 | 196702761 | 196702811 | 196702861 | 196702911 |
| 196703908 | 196703958 | 196704008 | 196704058 | 196704108 |
| 196703996 | 196704046 | 196704096 | 196704146 | 196704196 |
| 196704069 | 196704119 | 196704169 | 196704219 | 196704269 |
| 196704190 | 196704240 | 196704290 | 196704340 | 196704390 |
| 196705370 | 196705420 | 196705470 | 196705520 | 196705570 |
| 196706597 | 196706647 | 196706697 | 196706747 | 196706797 |
| 196706655 | 196706705 | 196706755 | 196706805 | 196706855 |
| 196707090 | 196707140 | 196707190 | 196707240 | 196707290 |
| 196707315 | 196707365 | 196707415 | 196707465 | 196707515 |
| 196707897 | 196707947 | 196707997 | 196708047 | 196708097 |
| 196773793 | 196773843 | 196773893 | 196773943 | 196773993 |
| 196774022 | 196774072 | 196774122 | 196774172 | 196774222 |
| 196774233 | 196774283 | 196774333 | 196774383 | 196774433 |
| 196774402 | 196774452 | 196774502 | 196774552 | 196774602 |
| 196774723 | 196774773 | 196774823 | 196774873 | 196774923 |
| 196774865 | 196774915 | 196774965 | 196775015 | 196775065 |
| 196775496 | 196775546 | 196775596 | 196775646 | 196775696 |
| 196776500 | 196776550 | 196776600 | 196776650 | 196776700 |
| 196776777 | 196776827 | 196776877 | 196776927 | 196776977 |
| 196839968 | 196840018 | 196840068 | 196840118 | 196840168 |
| 196840715 | 196840765 | 196840815 | 196840865 | 196840915 |
| 196841549 | 196841599 | 196841649 | 196841699 | 196841749 |
| 196841890 | 196841940 | 196841990 | 196842040 | 196842090 |
| 196842902 | 196842952 | 196843002 | 196843052 | 196843102 |
| 196843235 | 196843285 | 196843335 | 196843385 | 196843435 |
| 196843984 | 196844034 | 196844084 | 196844134 | 196844184 |
| 196851351 | 196851401 | 196851451 | 196851501 | 196851551 |
| 196853434 | 196853484 | 196853534 | 196853584 | 196853634 |
| 196853662 | 196853712 | 196853762 | 196853812 | 196853862 |
| 196880273 | 196880323 | 196880373 | 196880423 | 196880473 |
| 196985756 | 196985806 | 196985856 | 196985906 | 196985956 |
| 196985783 | 196985833 | 196985883 | 196985933 | 196985983 |
| 196987184 | 196987234 | 196987284 | 196987334 | 196987384 |
| 196988925 | 196988975 | 196989025 | 196989075 | 196989125 |
| 196989152 | 196989202 | 196989252 | 196989302 | 196989352 |
| 196989277 | 196989327 | 196989377 | 196989427 | 196989477 |
| 196989308 | 196989358 | 196989408 | 196989458 | 196989508 |
| 197453546 | 197453596 | 197453646 | 197453696 | 197453746 |
| 197453608 | 197453658 | 197453708 | 197453758 | 197453808 |
| 197454348 | 197454398 | 197454448 | 197454498 | 197454548 |
| 197454530 | 197454580 | 197454630 | 197454680 | 197454730 |
| 197454557 | 197454607 | 197454657 | 197454707 | 197454757 |
| 197454644 | 197454694 | 197454744 | 197454794 | 197454844 |
| 197454680 | 197454730 | 197454780 | 197454830 | 197454880 |
| 197454733 | 197454783 | 197454833 | 197454883 | 197454933 |
| 197454907 | 197454957 | 197455007 | 197455057 | 197455107 |
| 197454934 | 197454984 | 197455034 | 197455084 | 197455134 |
| 197456822 | 197456872 | 197456922 | 197456972 | 197457022 |
| 197457034 | 197457084 | 197457134 | 197457184 | 197457234 |
| 197457114 | 197457164 | 197457214 | 197457264 | 197457314 |
| 197457251 | 197457301 | 197457351 | 197457401 | 197457451 |
| 197457503 | 197457553 | 197457603 | 197457653 | 197457703 |
| 197459088 | 197459138 | 197459188 | 197459238 | 197459288 |
| 197487865 | 197487915 | 197487965 | 197488015 | 197488065 |
| 197488651 | 197488701 | 197488751 | 197488801 | 197488851 |
| 197488967 | 197489017 | 197489067 | 197489117 | 197489167 |
| 197524389 | 197524439 | 197524489 | 197524539 | 197524589 |
| 197524755 | 197524805 | 197524855 | 197524905 | 197524955 |
| 197525093 | 197525143 | 197525193 | 197525243 | 197525293 |
| 197525265 | 197525315 | 197525365 | 197525415 | 197525465 |
| 197525525 | 197525575 | 197525625 | 197525675 | 197525725 |
| 197525890 | 197525940 | 197525990 | 197526040 | 197526090 |
| 197526521 | 197526571 | 197526621 | 197526671 | 197526721 |
| 197526590 | 197526640 | 197526690 | 197526740 | 197526790 |
| 197527482 | 197527532 | 197527582 | 197527632 | 197527682 |
| 197527582 | 197527632 | 197527682 | 197527732 | 197527782 |
| 197528552 | 197528602 | 197528652 | 197528702 | 197528752 |
| 197556127 | 197556177 | 197556227 | 197556277 | 197556327 |
| 197609586 | 197609636 | 197609686 | 197609736 | 197609786 |
| 197609630 | 197609680 | 197609730 | 197609780 | 197609830 |
| 197611592 | 197611642 | 197611692 | 197611742 | 197611792 |
| 197611732 | 197611782 | 197611832 | 197611882 | 197611932 |
| 197611794 | 197611844 | 197611894 | 197611944 | 197611994 |
| 197613035 | 197613085 | 197613135 | 197613185 | 197613235 |
| 197613558 | 197613608 | 197613658 | 197613708 | 197613758 |
| 197614989 | 197615039 | 197615089 | 197615139 | 197615189 |
| 197615361 | 197615411 | 197615461 | 197615511 | 197615561 |
| 197631460 | 197631510 | 197631560 | 197631610 | 197631660 |
| 197631588 | 197631638 | 197631688 | 197631738 | 197631788 |
| 197632490 | 197632540 | 197632590 | 197632640 | 197632690 |
| 197632606 | 197632656 | 197632706 | 197632756 | 197632806 |
| 197633270 | 197633320 | 197633370 | 197633420 | 197633470 |
| 197633760 | 197633810 | 197633860 | 197633910 | 197633960 |
| 197638678 | 197638728 | 197638778 | 197638828 | 197638878 |
| 197638849 | 197638899 | 197638949 | 197638999 | 197639049 |
| 197639280 | 197639330 | 197639380 | 197639430 | 197639480 |
| 197651923 | 197651973 | 197652023 | 197652073 | 197652123 |
| 197652378 | 197652428 | 197652478 | 197652528 | 197652578 |
| 197653025 | 197653075 | 197653125 | 197653175 | 197653225 |
| 197654442 | 197654492 | 197654542 | 197654592 | 197654642 |
| 197687170 | 197687220 | 197687270 | 197687320 | 197687370 |
| 197687424 | 197687474 | 197687524 | 197687574 | 197687624 |
| 197688112 | 197688162 | 197688212 | 197688262 | 197688312 |
| 197688345 | 197688395 | 197688445 | 197688495 | 197688545 |
| 197688392 | 197688442 | 197688492 | 197688542 | 197688592 |
| 197692891 | 197692941 | 197692991 | 197693041 | 197693091 |
| 197692896 | 197692946 | 197692996 | 197693046 | 197693096 |
| 197694165 | 197694215 | 197694265 | 197694315 | 197694365 |
| 197695268 | 197695318 | 197695368 | 197695418 | 197695468 |
| 197695491 | 197695541 | 197695591 | 197695641 | 197695691 |
| 197695757 | 197695807 | 197695857 | 197695907 | 197695957 |
| 197696092 | 197696142 | 197696192 | 197696242 | 197696292 |
| 197696632 | 197696682 | 197696732 | 197696782 | 197696832 |
| 197696662 | 197696712 | 197696762 | 197696812 | 197696862 |
| 197697227 | 197697277 | 197697327 | 197697377 | 197697427 |
| 197697414 | 197697464 | 197697514 | 197697564 | 197697614 |
| 197698149 | 197698199 | 197698249 | 197698299 | 197698349 |
| 197698178 | 197698228 | 197698278 | 197698328 | 197698378 |
| 197708037 | 197708087 | 197708137 | 197708187 | 197708237 |
| 197708234 | 197708284 | 197708334 | 197708384 | 197708434 |
| 197757973 | 197758023 | 197758073 | 197758123 | 197758173 |
| 197760075 | 197760125 | 197760175 | 197760225 | 197760275 |
| 197761154 | 197761204 | 197761254 | 197761304 | 197761354 |
| 197761205 | 197761255 | 197761305 | 197761355 | 197761405 |
| 197776440 | 197776490 | 197776540 | 197776590 | 197776640 |
| 197777449 | 197777499 | 197777549 | 197777599 | 197777649 |
| 197777518 | 197777568 | 197777618 | 197777668 | 197777718 |
| 197778010 | 197778060 | 197778110 | 197778160 | 197778210 |
| 197781749 | 197781799 | 197781849 | 197781899 | 197781949 |
| 197781861 | 197781911 | 197781961 | 197782011 | 197782061 |
| 197784596 | 197784646 | 197784696 | 197784746 | 197784796 |
| 197785066 | 197785116 | 197785166 | 197785216 | 197785266 |
| 197785170 | 197785220 | 197785270 | 197785320 | 197785370 |
| 197786048 | 197786098 | 197786148 | 197786198 | 197786248 |
| 197786055 | 197786105 | 197786155 | 197786205 | 197786255 |
| 197787668 | 197787718 | 197787768 | 197787818 | 197787868 |
| 197805956 | 197806006 | 197806056 | 197806106 | 197806156 |
| 197806383 | 197806433 | 197806483 | 197806533 | 197806583 |
| 197812495 | 197812545 | 197812595 | 197812645 | 197812695 |
| 197813489 | 197813539 | 197813589 | 197813639 | 197813689 |
| 197813982 | 197814032 | 197814082 | 197814132 | 197814182 |
| 197840702 | 197840752 | 197840802 | 197840852 | 197840902 |
| 197840851 | 197840901 | 197840951 | 197841001 | 197841051 |
| 197855889 | 197855939 | 197855989 | 197856039 | 197856089 |
| 197859223 | 197859273 | 197859323 | 197859373 | 197859423 |
| 197860611 | 197860661 | 197860711 | 197860761 | 197860811 |
| 197861273 | 197861323 | 197861373 | 197861423 | 197861473 |
| 197895172 | 197895222 | 197895272 | 197895322 | 197895372 |
| 197902723 | 197902773 | 197902823 | 197902873 | 197902923 |
| 197902755 | 197902805 | 197902855 | 197902905 | 197902955 |
| 197902823 | 197902873 | 197902923 | 197902973 | 197903023 |
| 197903019 | 197903069 | 197903119 | 197903169 | 197903219 |
| 197903164 | 197903214 | 197903264 | 197903314 | 197903364 |
| 197903202 | 197903252 | 197903302 | 197903352 | 197903402 |
| 197903272 | 197903322 | 197903372 | 197903422 | 197903472 |
| 197903375 | 197903425 | 197903475 | 197903525 | 197903575 |
| 197903487 | 197903537 | 197903587 | 197903637 | 197903687 |
| 197903529 | 197903579 | 197903629 | 197903679 | 197903729 |
| 197903616 | 197903666 | 197903716 | 197903766 | 197903816 |
| 197903716 | 197903766 | 197903816 | 197903866 | 197903916 |
| 197903916 | 197903966 | 197904016 | 197904066 | 197904116 |
| 197904555 | 197904605 | 197904655 | 197904705 | 197904755 |
| 197906573 | 197906623 | 197906673 | 197906723 | 197906773 |
| 197907466 | 197907516 | 197907566 | 197907616 | 197907666 |
| 197907517 | 197907567 | 197907617 | 197907667 | 197907717 |
| 197907553 | 197907603 | 197907653 | 197907703 | 197907753 |
| 197907742 | 197907792 | 197907842 | 197907892 | 197907942 |
| 197909724 | 197909774 | 197909824 | 197909874 | 197909924 |
| 197948446 | 197948496 | 197948546 | 197948596 | 197948646 |
| 197948480 | 197948530 | 197948580 | 197948630 | 197948680 |
| 197948590 | 197948640 | 197948690 | 197948740 | 197948790 |
| 197948731 | 197948781 | 197948831 | 197948881 | 197948931 |
| 197948779 | 197948829 | 197948879 | 197948929 | 197948979 |
| 197973532 | 197973582 | 197973632 | 197973682 | 197973732 |
| 197974393 | 197974443 | 197974493 | 197974543 | 197974593 |
| 197994108 | 197994158 | 197994208 | 197994258 | 197994308 |
| 198023332 | 198023382 | 198023432 | 198023482 | 198023532 |
| 198023473 | 198023523 | 198023573 | 198023623 | 198023673 |

or the complement, or reverse complement of said polynucleic acid.

27. An (isolated) polynucleic acid comprising at least 15 contiguous nucleotides comprised in a region corresponding to a region flanked by any of the indicated 5′ and 3′ positions of the table below and comprising the nucleotide corresponding to the position of the indicated SNP referenced to maize chromosome 3, B73 AGPv4

| 5' (−100 nt) | 5' (−50 nt) | SNP | 3' (+50 nt) | 3' (+100 nt) |
|---|---|---|---|---|
| 197453608 | 197453658 | 197453708 | 197453758 | 197453858 |
| 197454530 | 197454580 | 197454630 | 197454680 | 197454780 |
| 197454733 | 197454783 | 197454833 | 197454883 | 197454983 |
| 197456822 | 197456872 | 197456922 | 197456972 | 197457072 |
| 197488651 | 197488701 | 197488751 | 197488801 | 197488901 |
| 197524389 | 197524439 | 197524489 | 197524539 | 197524639 |
| 197525525 | 197525575 | 197525625 | 197525675 | 197525775 |
| 197525890 | 197525940 | 197525990 | 197526040 | 197526140 |
| 197556127 | 197556177 | 197556227 | 197556277 | 197556377 |
| 197611794 | 197611844 | 197611894 | 197611944 | 197612044 |
| 197613035 | 197613085 | 197613135 | 197613185 | 197613285 |
| 197613558 | 197613608 | 197613658 | 197613708 | 197613808 |
| 197614989 | 197615039 | 197615089 | 197615139 | 197615239 |
| 197615361 | 197615411 | 197615461 | 197615511 | 197615611 |
| 197633760 | 197633810 | 197633860 | 197633910 | 197634010 |
| 197696092 | 197696142 | 197696192 | 197696242 | 197696342 |

or the complement, or reverse complement of said polynucleic acid.

29. An (isolated) polynucleic acid comprising at least 15 contiguous nucleotides comprised in a region corresponding to a region flanked by any of the indicated 5′ and 3′ positions of the table below and comprising the nucleotide corresponding to the position of the indicated SEQ ID NO

| SEQ | | | | | |
|---|---|---|---|---|---|
| ID | 5' | 5' | poly- | 3' | 3' |
| NO | (−100 nt) | (−50 nt) | morphism | (+50 nt) | (+100 nt) |
| 1 | -65 | −15 | 35 | 85 | 135 |
| 1 | 304 | 354 | 404 | 454 | 504 |
| 1 | 344 | 394 | 444-452 | 502 | 552 |
| 1 | 363 | 413 | 463 | 513 | 563 |
| 1 | 437 | 487 | 537 | 587 | 637 |
| 1 | 635 | 685 | 735 | 785 | 835 |
| 1 | 648 | 698 | 748-759 | 809 | 859 |
| 1 | 661 | 711 | 761 | 811 | 861 |
| 1 | 697 | 747 | 797 | 847 | 897 |
| 1 | 948 | 998 | 1048 | 1098 | 1148 |
| 1 | 956 | 1006 | 1056 | 1106 | 1156 |
| 1 | 956 | 1015 | 1065-1066 | 1116 | 1166 |
| 1 | 972 | 1022 | 1072-1073 | 1123 | 1173 |
| 1 | 971 | 1021 | 1071 | 1121 | 1171 |
| 1 | 1088 | 1138 | 1188 | 1238 | 1288 |
| 1 | 1118 | 1168 | 1218 | 1268 | 1318 |
| 1 | 1665 | 1715 | 1765 | 1815 | 1865 |
| 1 | 1669 | 1719 | 1769 | 1819 | 1869 |
| 1 | 1744 | 1794 | 1844 | 1894 | 1944 |
| 1 | 1756 | 1806 | 1856 | 1906 | 1956 |
| 1 | 1976 | 2026 | 2076 | 2126 | 2176 |
| 1 | 1989 | 2039 | 2089 | 2139 | 2189 |
| 1 | 2046 | 2096 | 2146 | 2196 | 2246 |
| 1 | 2068 | 2118 | 2168-2169 | 2219 | 2269 |
| 1 | 2114 | 2164 | 2214 | 2264 | 2314 |
| 1 | 2270 | 2320 | 2370 | 2420 | 2470 |
| 1 | 2482 | 2532 | 2582 | 2632 | 2682 |
| 1 | 2532 | 2582 | 2632-2637 | 2687 | 2737 |
| 1 | 2541 | 2591 | 2641 | 2691 | 2741 |
| 1 | 2543 | 2593 | 2643-2644 | 2694 | 2744 |
| 1 | 2596 | 2646 | 2696 | 2746 | 2796 |
| 1 | 2638 | 2688 | 2738 | 2788 | 2838 |
| 1 | 2743 | 2793 | 2843 | 2893 | 2943 |
| 1 | 2749 | 2799 | 2849 | 2899 | 2949 |
| 1 | 2854 | 2904 | 2954-2955 | 3005 | 3055 |
| 1 | 2904 | 2954 | 3004 | 3054 | 3104 |
| 1 | 2947 | 2997 | 3047 | 3097 | 3147 |
| 1 | 2968 | 3018 | 3068 | 3118 | 3168 |
| 1 | 3118 | 3168 | 3218 | 3268 | 3318 |
| 5 | 386 | 436 | 486 | 536 | 586 |
| 5 | 511 | 561 | 611 | 661 | 711 |
| 5 | 538 | 588 | 638 | 688 | 738 |
| 5 | 589 | 639 | 689 | 739 | 789 |
| 5 | 712 | 762 | 812 | 862 | 912 |
| 5 | 765 | 815 | 865 | 915 | 965 |
| 5 | 801 | 851 | 901 | 951 | 1001 |
| 5 | 888 | 938 | 988 | 1038 | 1088 |
| 5 | 915 | 965 | 1015 | 1065 | 1115 |
| 5 | 985 | 1035 | 1085 | 1135 | 1185 |
| 5 | 1097 | 1147 | 1197 | 1247 | 1297 |
| 5 | 1245 | 1295 | 1345 | 1395 | 1445 |
| 5 | 1361 | 1411 | 1461 | 1511 | 1561 |
| 5 | 1837 | 1887 | 1937 | 1987 | 2037 |
| 5 | 1899 | 1949 | 1999 | 2049 | 2099 |
| 5 | 2013 | 2063 | 2113-2115 | 2165 | 2215 |
| 5 | 2186 | 2236 | 2286 | 2336 | 2386 |
| 5 | 2193 | 2243 | 2293-2297 | 2347 | 2397 |
| 5 | 2299 | 2349 | 2399 | 2449 | 2499 |
| 5 | 2348 | 2398 | 2448-2450 | 2500 | 2550 |
| 5 | 2722 | 2772 | 2822-2823 | 2873 | 2923 |
| 5 | 2756 | 2806 | 2856 | 2906 | 2956 |
| 5 | 2826 | 2876 | 2926 | 2976 | 3026 |
| 5 | 2898 | 2948 | 2998 | 3048 | 3098 |
| 5 | 2929 | 2979 | 3029 | 3079 | 3129 |
| 5 | 2985 | 3035 | 3085 | 3135 | 3185 |
| 5 | 3002 | 3052 | 3102 | 3152 | 3202 |
| 5 | 3012 | 3062 | 3112 | 3162 | 3212 |
| 5 | 3020 | 3070 | 3120 | 3170 | 3220 |
| 5 | 3068 | 3118 | 3168 | 3218 | 3268 |
| 9 | 292 | 342 | 392-393 | 443 | 493 |
| 9 | 450 | 500 | 550 | 600 | 650 |
| 9 | 491 | 541 | 591 | 641 | 691 |
| 9 | 786 | 836 | 886-887 | 937 | 987 |
| 9 | 834 | 884 | 934 | 984 | 1034 |
| 9 | 857 | 907 | 957 | 1007 | 1057 |
| 9 | 997 | 1047 | 1097 | 1147 | 1197 |
| 9 | 1030 | 1080 | 1130 | 1180 | 1230 |
| 9 | 1195 | 1245 | 1295 | 1345 | 1395 |
| 9 | 1362 | 1412 | 1462 | 1512 | 1562 |
| 9 | 1367 | 1417 | 1467 | 1517 | 1567 |
| 9 | 1441 | 1491 | 1541 | 1591 | 1641 |
| 9 | 1484 | 1534 | 1584 | 1634 | 1684 |
| 9 | 1514 | 1564 | 1614 | 1664 | 1714 |
| 9 | 1613 | 1663 | 1713 | 1763 | 1813 |
| 9 | 1674 | 1724 | 1774 | 1824 | 1874 |
| 9 | 1695 | 1745 | 1795-1796 | 1846 | 1896 |
| 9 | 1715 | 1765 | 1815 | 1865 | 1915 |
| 9 | 1794 | 1844 | 1894-1895 | 1945 | 1995 |
| 9 | 1810 | 1860 | 1910 | 1960 | 2010 |
| 9 | 1854 | 1904 | 1954-1955 | 2005 | 2055 |
| 9 | 1900 | 1950 | 2000 | 2050 | 2100 |
| 9 | 1960 | 2010 | 2060 | 2110 | 2160 |
| 9 | 2081 | 2131 | 2181 | 2231 | 2281 |
| 9 | 2254 | 2304 | 2354 | 2404 | 2454 |
| 9 | 2272 | 2322 | 2372 | 2422 | 2472 |
| 9 | 2294 | 2344 | 2394-2399 | 2449 | 2499 |
| 9 | 2328 | 2378 | 2428 | 2478 | 2528 |
| 9 | 2339 | 2389 | 2439-2440 | 2490 | 2540 |
| 13 | 551 | 601 | 651-652 | 702 | 752 |
| 13 | 707 | 757 | 807 | 857 | 907 |

or the complement, or reverse complement of said polynucleic acid.

30. An (isolated) polynucleic acid comprising at least 15 contiguous nucleotides comprised in a region corresponding to a region flanked by any of the indicated 5′ and 3′ positions of the table below and comprising the nucleotide corresponding to the position of the indicated SEQ ID NO

| SEQ | | | | | |
|---|---|---|---|---|---|
| ID | 5' | 5' | poly- | 3' | 3' |
| NO | (−100 nt) | (−50 nt) | morphism | (+50 nt) | (+100 nt) |
| 2 | −65 | −15 | 35 | 85 | 135 |
| 2 | 304 | 354 | 404 | 454 | 504 |
| 2 | 343 | 393 | 443-444 | 494 | 544 |
| 2 | 354 | 404 | 454 | 504 | 554 |
| 2 | 428 | 478 | 528 | 578 | 628 |
| 2 | 626 | 676 | 726 | 776 | 826 |
| 2 | 638 | 688 | 738-739 | 789 | 839 |
| 2 | 640 | 690 | 740 | 790 | 840 |
| 2 | 676 | 726 | 776 | 826 | 876 |
| 2 | 927 | 977 | 1027 | 1077 | 1127 |
| 2 | 935 | 985 | 1035 | 1085 | 1135 |
| 2 | 945 | 995 | 1045 | 1095 | 1145 |
| 2 | 953 | 1003 | 1053-1059 | 1109 | 1159 |
| 2 | 958 | 1008 | 1058 | 1108 | 1158 |
| 2 | 1075 | 1125 | 1175 | 1225 | 1275 |
| 2 | 1108 | 1158 | 1208 | 1258 | 1308 |
| 2 | 1657 | 1707 | 1757 | 1807 | 1857 |
| 2 | 1661 | 1711 | 1761 | 1811 | 1861 |
| 2 | 1736 | 1786 | 1836 | 1886 | 1936 |
| 2 | 1748 | 1798 | 1848 | 1898 | 1948 |
| 2 | 1968 | 2018 | 2068 | 2118 | 2168 |
| 2 | 1981 | 2031 | 2081 | 2131 | 2181 |
| 2 | 2038 | 2088 | 2138 | 2188 | 2238 |
| 2 | 2061 | 2111 | 2161 | 2211 | 2261 |
| 2 | 2107 | 2157 | 2207 | 2257 | 2307 |
| 2 | 2262 | 2312 | 2362-2363 | 2413 | 2463 |
| 2 | 2474 | 2524 | 2574 | 2624 | 2674 |
| 2 | 2524 | 2574 | 2624-2629 | 2679 | 2729 |
| 2 | 2533 | 2583 | 2633 | 2683 | 2733 |
| 2 | 2535 | 2585 | 2635-2636 | 2686 | 2736 |
| 2 | 2588 | 2638 | 2688 | 2738 | 2788 |
| 2 | 2630 | 2680 | 2730 | 2780 | 2830 |
| 2 | 2734 | 2784 | 2834-2835 | 2885 | 2935 |
| 2 | 2740 | 2790 | 2840 | 2890 | 2940 |
| 2 | 2846 | 2896 | 2946-2947 | 2997 | 3047 |
| 2 | 2897 | 2947 | 2997 | 3047 | 3097 |
| 2 | 2940 | 2990 | 3040 | 3090 | 3140 |
| 2 | 2961 | 3011 | 3061 | 3111 | 3161 |
| 2 | 3111 | 3161 | 3211 | 3261 | 3311 |
| 6 | 386 | 436 | 486 | 536 | 586 |
| 6 | 511 | 561 | 611 | 661 | 711 |
| 6 | 538 | 588 | 638 | 688 | 738 |
| 6 | 589 | 639 | 689 | 739 | 789 |
| 6 | 712 | 762 | 812 | 862 | 912 |
| 6 | 765 | 815 | 865 | 915 | 965 |
| 6 | 801 | 851 | 901 | 951 | 1001 |
| 6 | 888 | 938 | 988 | 1038 | 1088 |
| 6 | 915 | 965 | 1015 | 1065 | 1115 |
| 6 | 985 | 1035 | 1085 | 1135 | 1185 |
| 6 | 1097 | 1147 | 1197 | 1247 | 1297 |
| 6 | 1245 | 1295 | 1345 | 1395 | 1445 |
| 6 | 1361 | 1411 | 1461 | 1511 | 1561 |
| 6 | 1837 | 1887 | 1937 | 1987 | 2037 |
| 6 | 1899 | 1949 | 1999 | 2049 | 2099 |
| 6 | 2012 | 2062 | 2112-2113 | 2163 | 2213 |
| 6 | 2183 | 2233 | 2283 | 2333 | 2383 |
| 6 | 2189 | 2239 | 2289-2290 | 2340 | 2390 |
| 6 | 2291 | 2341 | 2391 | 2441 | 2491 |
| 6 | 2339 | 2389 | 2439-2440 | 2490 | 2540 |
| 6 | 2710 | 2760 | 2810-2811 | 2861 | 2911 |
| 6 | 2743 | 2793 | 2843 | 2893 | 2943 |
| 6 | 2813 | 2863 | 2913 | 2963 | 3013 |
| 6 | 2885 | 2935 | 2985 | 3035 | 3085 |
| 6 | 2916 | 2966 | 3016 | 3066 | 3116 |
| 6 | 2972 | 3022 | 3072 | 3122 | 3172 |
| 6 | 2989 | 3039 | 3089 | 3139 | 3189 |
| 6 | 2999 | 3049 | 3099 | 3149 | 3199 |
| 6 | 3007 | 3057 | 3107 | 3157 | 3207 |
| 6 | 3055 | 3105 | 3155 | 3205 | 3255 |
| 10 | 293 | 343 | 393-397 | 447 | 497 |
| 10 | 455 | 505 | 555 | 605 | 655 |
| 10 | 496 | 546 | 596 | 646 | 696 |
| 10 | 791 | 841 | 891-892 | 942 | 992 |
| 10 | 839 | 889 | 939 | 989 | 1039 |
| 10 | 862 | 912 | 962 | 1012 | 1062 |
| 10 | 1002 | 1052 | 1102 | 1152 | 1202 |
| 10 | 1035 | 1085 | 1135 | 1185 | 1235 |
| 10 | 1200 | 1250 | 1300 | 1350 | 1400 |
| 10 | 1367 | 1417 | 1467 | 1517 | 1567 |
| 10 | 1372 | 1422 | 1472 | 1522 | 1572 |
| 10 | 1446 | 1496 | 1546 | 1596 | 1646 |
| 10 | 1503 | 1553 | 1603 | 1653 | 1703 |
| 10 | 1533 | 1583 | 1633 | 1683 | 1733 |
| 10 | 1632 | 1682 | 1732 | 1782 | 1832 |
| 10 | 1693 | 1743 | 1793 | 1843 | 1893 |
| 10 | 1715 | 1765 | 1815-1823 | 1873 | 1923 |
| 10 | 1743 | 1793 | 1843 | 1893 | 1943 |
| 10 | 1823 | 1873 | 1923 | 1973 | 2023 |
| 10 | 1839 | 1889 | 1939 | 1989 | 2039 |
| 10 | 1884 | 1934 | 1984-1991 | 2041 | 2091 |
| 10 | 1937 | 1987 | 2037 | 2087 | 2137 |
| 10 | 1997 | 2047 | 2097 | 2147 | 2197 |
| 10 | 2118 | 2168 | 2218 | 2268 | 2318 |
| 10 | 2291 | 2341 | 2391 | 2441 | 2491 |
| 10 | 2309 | 2359 | 2409 | 2459 | 2509 |
| 10 | 2331 | 2381 | 2431-2434 | 2484 | 2534 |
| 10 | 2363 | 2413 | 2463 | 2513 | 2563 |
| 10 | 2375 | 2425 | 2475-2476 | 2526 | 2576 |
| 14 | 552 | 602 | 652-654 | 704 | 754 |
| 14 | 710 | 760 | 810 | 860 | 910 |

or the complement, or reverse complement of said polynucleic acid.

31. The (isolated) polynucleic acid according to any of statements 25 to 30, comprising at most 500 nucleotides, preferably at most 200 nucleotides, more preferably at most 100 nucleotides, most preferably at most 50 nucleotides, such as at most 35 nucleotides.

32. An (isolated) polynucleic acid specifically hybridizing with the polynucleic acid according to any of statements 25 to 31, or the complement or reverse complement of said polynucleic acid.

33. The (isolated) polynucleic acid according to any of statements 25 to 32, which is a primer or a probe.

34. The (isolated) polynucleotide according to any of statements 25 to 33, which is an allele-specific primer or probe.

35. The (isolated) polynucleic acid according to any of statements 25 to 34 which is a KASP primer.

36. A primer or a probe comprising the (isolated) polynucleic acid according to any of statements 25 to 35.

37. The primer according to statement 36, which is an allele-specific primer.

38. The primer according to statement 36 or 37, which is a KASP primer.

39. A primer specifically hybridizing with a molecular marker (allele) of Table 4 or Table 5, or the complement or reverse complement thereof.

40. A primer capable of specifically detecting a molecular marker (allele) of Table 4 or Table 5.

41. A primer set capable of specifically detecting a molecular marker (allele) of Table 4 or Table 5.

42. A primer set capable of amplifying a polynucleic acid comprising a molecular marker (allele) of Table 4 or Table 5.

43. A (maize) plant or plant part comprising one or more molecular marker (allele) of Table 4 or Table 5, the locus as defined in any of statements 1 to 10, and/or a polynucleic acid as defined in any of statements 5, 6, 9, 10, or 25 to 30.

44. A method for generating a (maize) plant or plant part, comprising introducing in the genome of said plant or plant part a locus as defined in any of statements 1 to 10, or a (functional) fragment thereof.

45. The method according to statement 44, wherein introducing into the genome comprises transgenesis.

44. The method according to statements 44 or 45, wherein introducing into the genome comprises introgression.

47. The method according to any of statements 44 to 46, comprising transforming a plant or plant part, preferably a plant cell, more preferably a protoplast, with a polynucleic acid encoding a locus as defined in any of statements 1 to 10, and optionally regenerating a plant from said plant cell, preferably protoplast.

48. The method according to statement 47, wherein said polynucleic acid which is introduced has a different sequence than a corresponding polynucleic acid of the plant.

49. A method for generating a (maize) plant or plant part, comprising (a) providing a first (maize) plant identified according to any of statements 1 to 24 or generated according to any of statements 40 to 44, (b) crossing said first (maize) plant with a second (maize) plant having cytoplasmic male sterility; and optionally (d) harvesting said (maize) plant part from the progeny.

50. The method according to any of statements 1 to 24 or 44 to 49, wherein said plant part is a cell, tissue or organ.

51. The method according to any of statements 1 to 24 or 44 to 50, wherein said plant part is a protoplast.

52. The method according to any of statements 1 to 24 or 44 to 50, wherein said plant part is a seed.

53. The method according to any of statements 1 to 24, wherein said locus, polynucleic acid, or molecular marker (allele) is homozygous.

54. The method according to any of statements 1 to 24, wherein said locus, polynucleic acid, or molecular marker (allele) is heterozygous.

55. Use of a polynucleic acid, primer or probe, or primer set according to any of statements 5, 6, 9, 10, or 25 to 42 for identifying a (maize) plant or plant part.

56. Use of a polynucleic acid according to any of statements 5, 6, 9, 10, or 25 to 30, or a locus as defined in any of statements 1 to 10, for generating a (maize) plant or plant part.

57. The method according to any of statements 9 to 17, or 22 to 24, wherein the sequence which is at least 80%, preferably at least 85%, more preferably at least 90%, most preferably at least 95% identical to a sequence as set forth in any one or more of SEQ ID NOs: 1, 5, 9, or 13 comprises on or more, preferably all, of the respective associated (restorer) polymorphism(s) as listed in Table 5.

58. The method according to any of statements 9 to 17, or 22 to 24, wherein the sequence which is at least 80%, preferably at least 85%, more preferably at least 90%, most preferably at least 95% identical to a sequence as set forth in any one or more of SEQ ID NOs: 2, 6, 10, or 14 comprises on or more, preferably all, of the respective associated (maintainer) polymorphism(s) as listed in Table 5.

59. The method according to any of statements 1 to 24 or 57 to 58, which is a method for discriminating between a maize plant or plant part having said (haplotype associated with) cytoplasmic male sterility (fertility) restorer locus on chromosome 3 (RF-03-01) and a maize plant lacking said (haplotype associated with) cytoplasmic male sterility (fertility) restorer locus on chromosome 3 (RF-03-01).

60. The method according to any of statements 1 to 17 or 57 to 59, wherein said maize plant or plant part is identified as having said (haplotype associated with) cytoplasmic male sterility (fertility) restorer locus on chromosome 3 (RF-03-01) if said (haplotype associated with) cytoplasmic male sterility (fertility) restorer locus on chromosome 3 (RF-03-01) is detected.

61. The method according to any of statements 1 to 17 or 57 to 59, wherein said maize plant or plant part is identified as lacking said (haplotype associated with) cytoplasmic male sterility (fertility) restorer locus on chromosome 3 (RF-03-01) if said (haplotype associated with) cytoplasmic male sterility (fertility) restorer locus on chromosome 3 (RF-03-01) is not detected.

62. The method according to any of statements 18 or 59, wherein said maize plant or plant part is identified as having said (haplotype associated with) cytoplasmic male sterility (fertility) restorer locus on chromosome 3 (RF-03-01) if any one or more molecular marker allele associated with said restorer locus in Table 4 or 5 is identified.

63. The method according to any of statements 18 or 59, wherein said maize plant or plant part is identified as lacking said (haplotype associated with) cytoplasmic male sterility (fertility) restorer locus on chromosome 3 (RF-03-01) if any one or more molecular marker allele associated with said restorer locus in Table 4 or 5 is not identified.

64. The method according to any of statements 18 or 59, wherein said maize plant or plant part is identified as lacking said (haplotype associated with) cytoplasmic male sterility (fertility) restorer locus on chromosome 3 (RF-03-01) if any one or more molecular marker allele associated with the maintainer locus in Table 4 or 5 is identified.

65. The method according to any of statements 19 or 59, wherein said maize plant or plant part is identified as having said (haplotype associated with) cytoplasmic male sterility (fertility) restorer locus on chromosome 3 (RF-03-01) if any one or more of SEQ ID NOs: 17 to 200 having the restorer SNP is identified.

66. The method according to any of statements 19 or 59, wherein said maize plant or plant part is identified as lacking said (haplotype associated with) cytoplasmic male sterility (fertility) restorer locus on chromosome 3 (RF-03-01) if any one or more of SEQ ID NOs: 17 to 200 not having the restorer SNP is identified.

67. The method according to any of statements 19 or 59, wherein said maize plant or plant part is identified as lacking said (haplotype associated with) cytoplasmic male sterility (fertility) restorer locus on chromosome 3 (RF-03-01) if any one or more of SEQ ID NOs: 17 to 200 having the maintainer SNP is identified.

68. The method according to any of statements 20 or 59, wherein said maize plant or plant part is identified as having said (haplotype associated with) cytoplasmic male sterility (fertility) restorer locus on chromosome 3 (RF-03-01) if any one or more of SEQ ID NOs: 68 to 140 having the restorer SNP is identified.

69. The method according to any of statements 20 or 59, wherein said maize plant or plant part is identified as lacking said (haplotype associated with) cytoplasmic male sterility (fertility) restorer locus on chromosome 3 (RF-03-01) if any one or more of SEQ ID NOs: 68 to 140 not having the restorer SNP is identified.

70. The method according to any of statements 20 or 59, wherein said maize plant or plant part is identified as lacking said (haplotype associated with) cytoplasmic male sterility (fertility) restorer locus on chromosome 3 (RF-03-01) if any one or more of SEQ ID NOs: 68 to 140 having the maintainer SNP is identified.

71. The method according to any of statements 21 or 59, wherein said maize plant or plant part is identified as having said (haplotype associated with) cytoplasmic male sterility (fertility) restorer locus on chromosome 3 (RF-03-01) if any one or more of SEQ ID NOs: 70, 72, 76, 79, 86, 88, 92, 93, 99, 104, 105, 107, 108, 109, 115, and 134 having the restorer SNP is identified.

72. The method according to any of statements 21 or 59, wherein said maize plant or plant part is identified as lacking said (haplotype associated with) cytoplasmic male sterility (fertility) restorer locus on chromosome 3 (RF-03-01) if any one or more of SEQ ID NOs: 70, 72, 76, 79, 86, 88, 92, 93, 99, 104, 105, 107, 108, 109, 115, and 134 not having the restorer SNP is identified.

73. The method according to any of statements 21 or 59, wherein said maize plant or plant part is identified as lacking said (haplotype associated with) cytoplasmic male sterility (fertility) restorer locus on chromosome 3 (RF-03-01) if any one or more of SEQ ID NOs: 70, 72, 76, 79, 86, 88, 92, 93, 99, 104, 105, 107, 108, 109, 115, and 134 having the maintainer SNP is identified.

74. The method according to any of statements 22, 24, or 57 to 59, wherein said maize plant or plant part is identified as having said (haplotype associated with) cytoplasmic male sterility (fertility) restorer locus on chromosome 3 (RF-03-01) if any one or more of SEQ ID NOs: 1, 5, 9, or 13, or a polynucleic acid comprising a sequence which is at least 80%, preferably at least 85%, more preferably at least 90%, most preferably at least 95% identical to a sequence as set forth in any one or more of SEQ ID NOs: 1, 5, 9 or 13 is identified.

75. The method according to any of statements 22, 24, or 57 to 59, wherein said maize plant or plant part is identified as lacking said (haplotype associated with) cytoplasmic male sterility (fertility) restorer locus on chromosome 3 (RF-03-01) if any one or more of SEQ ID NOs: 1, 5, 9, or 13, or a polynucleic acid comprising a sequence which is at least 80%, preferably at least 85%, more preferably at least 90%, most preferably at least 95% identical to a sequence as set forth in any one or more of SEQ ID NOs: 1, 5, 9 or 13 is not identified.

76. The method according to any of statements 22, 24, or 57 to 59, wherein said maize plant or plant part is identified as lacking said (haplotype associated with) cytoplasmic male sterility (fertility) restorer locus on chromosome 3 (RF-03-01) if any one or more of SEQ ID NOs: 2, 6, 10, or 14, or a polynucleic acid comprising a sequence which is at least 80%, preferably at least 85%, more preferably at least 90%, most preferably at least 95% identical to a sequence as set forth in any one or more of SEQ ID NOs: 2, 6, 10 or 14 is identified.

77. The method according to any of statements 23 or 57 to 59, wherein said maize plant or plant part is identified as having said (haplotype associated with) cytoplasmic male sterility (fertility) restorer locus on chromosome 3 (RF-03-01) if SEQ ID NO: 1 or a polynucleic acid comprising a sequence which is at least 80%, preferably at least 85%, more preferably at least 90%, most preferably at least 95% identical to a sequence as set forth in SEQ ID NOs: 1 is identified.

78. The method according to any of statements 23 or 57 to 59, wherein said maize plant or plant part is identified as lacking said (haplotype associated with) cytoplasmic male sterility (fertility) restorer locus on chromosome 3 (RF-03-01) if SEQ ID NO: 1 or a polynucleic acid comprising a sequence which is at least 80%, preferably at least 85%, more preferably at least 90%, most preferably at least 95% identical to a sequence as set forth in SEQ ID NOs: 1 is not identified.

79. The method according to any of statements 23 or 57 to 59, wherein said maize plant or plant part is identified as lacking said (haplotype associated with) cytoplasmic male sterility (fertility) restorer locus on chromosome 3 (RF-03-01) if SEQ ID NO: 2 or a polynucleic acid comprising a sequence which is at least 80%, preferably at least 85%, more preferably at least 90%, most preferably at least 95% identical to a sequence as set forth in SEQ ID NOs: 2 is identified.

80. A (isolated) polynucleic acid having a sequence as set forth in any of SEQ ID NOs: 1, 5, 9, or 13, or a (unique) fragment thereof, or a polynucleic acid comprising a sequence which is at least 80%, preferably at least 85%, more preferably at least 90%, most preferably at least 95% identical to a sequence as set forth in any one or more of SEQ ID NOs: 1, 5, 9 and 13; or one or more of SEQ ID NOs: 2, 6, 10, and 14, or a (unique) fragment thereof, or a polynucleic acid comprising a sequence which is at least 80%, preferably at least 85%, more preferably at least 90%, most preferably at least 95% identical to a sequence as set forth in any one or more of SEQ ID NOs: 2, 6, 10, and 14, or a (unique) fragment thereof.

81. A (isolated) polynucleic acid of
Zm00001d043358
   having a genomic sequence of SEQ ID NO: 1 or 2, or a genomic sequence which is at least 80%, preferably at least 85%, more preferably at least 90%, most preferably at least 95% identical to SEQ ID NO: 1 or 2;
   having a coding sequence of SEQ ID NO: 201 or 3, or a coding sequence which is at least 80%, preferably at least 85%, more preferably at least 90%, most preferably at least 95% identical to SEQ ID NO: 201 or 3; and/or
   encoding a protein having a sequence of SEQ ID NO: 202 or 4, or a protein having a sequence which is at least 80%, preferably at least 85%, more preferably at least 90%, most preferably at least 95% identical to SEQ ID NO: 202 or 4;
Zm00001d043352
   having a genomic sequence of SEQ ID NO: 5 or 6, or a genomic sequence which is at least 80%, preferably at least 85%, more preferably at least 90%, most preferably at least 95% identical to SEQ ID NO: 5 or 6;
   having a coding sequence of SEQ ID NO: 203 or 7, or a coding sequence which is at least 80%, preferably at least 85%, more preferably at least 90%, most preferably at least 95% identical to SEQ ID NO: 203 or 7; and/or
   encoding a protein having a sequence of SEQ ID NO: 204 or 8, or a protein having a sequence which is at least 80%, preferably at least 85%, more preferably at least 90%, most preferably at least 95% identical to SEQ ID NO: 204 or 8;
Zm00001d043356
   having a genomic sequence of SEQ ID NO: 9 or 10, or a genomic sequence which is at least 80%, preferably at least 85%, more preferably at least 90%, most preferably at least 95% identical to SEQ ID NO: 9 or 10;
   having a coding sequence of SEQ ID NO: 205 or 11, or a coding sequence which is at least 80%, preferably at least 85%, more preferably at least 90%, most preferably at least 95% identical to SEQ ID NO: 205 or 11; and/or
   encoding a protein having a sequence of SEQ ID NO: 206 or 12, or a protein having a sequence which is at least 80%, preferably at least 85%, more preferably at least 90%, most preferably at least 95% identical to SEQ ID NO: 206 or 12; or
Zm00001d043357
   having a genomic sequence of SEQ ID NO: 13 or 14, or a genomic sequence which is at least 80%, preferably at least 85%, more preferably at least 90%, most preferably at least 95% identical to SEQ ID NO: 13 or 14;
   having a coding sequence of SEQ ID NO: 207 or 15, or a coding sequence which is at least 80%, preferably at least 85%, more preferably at least 90%, most preferably at least 95% identical to SEQ ID NO: 207 or 15; and/or
   encoding a protein having a sequence of SEQ ID NO: 208 or 16, or a protein having a sequence which is at least 80%, preferably at least 85%, more preferably at least 90%, most preferably at least 95% identical to SEQ ID NO: 208 or 16.

82. The polynucleic acid according to statement 80 or 81, wherein the sequence which is at least 80%, preferably at least 85%, more preferably at least 90%, most preferably at least 95% identical to the recited SEQ ID NOs comprises on or more, preferably all, of the respective associated (restorer) polymorphism(s) as listed in Table 5.

83. The polynucleic acid according to any of statement 80 or 81, wherein the sequence which is at least 80%, preferably at least 85%, more preferably at least 90%, most preferably at least 95% identical to the recited SEQ ID NOs comprises on or more, preferably all, of the respective associated (maintainer) polymorphism(s) as listed in Table 5.

84. The method according to statement 44 to 54, comprising introducing in the genome of said plant or plant part a locus as defined in any of statements 1 to 17 or a polynucleic acid as defined in any of statements 80 to 83, or a (functional) fragment thereof.

85. The method according to any of statements 44 to 54 or 84, wherein said polynucleic acid is a genomic polynucleic acid flanked by molecular markers ma0016fm86 and ma0004tr23.

86. The method according to statement 44 to 54 or 84, wherein said polynucleic acid is a genomic polynucleic acid flanked by molecular markers ma0000sa77 and ma0016fu05.

87. The method according to any of statements 44 to 54 or 84 to 86, wherein said locus or polynucleic acid comprises the maintainer polymorphisms of Table 5.

88. The method according to any of statements 44 to 54 or 84 to 87, wherein said locus or polynucleic acid comprises the maintainer polymorphisms of Table 4.

89. The method according to any of statements 44 to 54 or 84 to 86, wherein said locus or polynucleic acid comprises the restorer polymorphisms of Table 5.

90. The method according to any of statements 44 to 54 or 84 to 86 or 89, wherein said locus or polynucleic acid comprises the restorer polymorphisms of Table 4.

91. The method according to any of statements 44 to 54 or 84 to 90, comprising crossing a first maize plant and a second maize plant, and selecting offspring comprising a locus or polynucleic acid as defined in any of the previous statements.

92. The method according to statement 91, comprising selecting offspring not comprising a restorer locus or polynucleic acid comprising the restorer polymorphisms or SNPs as defined in any of the statements.

93. The method according to claim 91 or 92, wherein said first or second maize plant is a cytoplasmic male sterile maize plant.

94. Use of a polynucleic acid according to any of claims 25 to 42 or 80 to 83 for identifying a maize plant or plant part or for generating a maize plant or plant part.

95. Use of a polynucleic acid according to any of claims 25 to 42 or 80 to 83 for identifying a maize plant or plant part.

96. Use of a polynucleic acid according to any of claims 80 to 83 for generating a maize plant or plant part.

### BRIEF DESCRIPTION OF THE FIGURES

FIG. 1: GWAS analysis using rf4-lines based on Illumina Chip data (35K)

FIG. 2: Sequence alignment of the genomic sequence of Zm00001d043358 of the reference B73 genome (SEQ ID NO: 2) and Zm00001d043358 of an embodiment of the present invention (SEQ ID NO: 1).

FIG. 3: Sequence alignment of the genomic sequence of Zm00001d043352 of the reference B73 genome (SEQ ID NO: 6) and Zm00001d043352 of an embodiment of the present invention (SEQ ID NO: 5).

FIG. 4: Sequence alignment of the genomic sequence of Zm00001d043356 of the reference B73 genome (SEQ ID NO: 10) and Zm00001d043356 of an embodiment of the present invention (SEQ ID NO: 9).

FIG. 5: Sequence alignment of the genomic sequence of Zm00001d043357 of the reference B73 genome (SEQ ID NO: 14) and Zm00001d043357 of an embodiment of the present invention (SEQ ID NO: 13).

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

The term “plant” includes whole plants, including descendants or progeny thereof. As used herein unless clearly indicated otherwise, the term “plant” intends to mean a plant at any developmental stage. The term “plant part” includes any part or derivative of the plant, including particular plant tissues or structures, plant cells, plant protoplast, plant cell or tissue culture from which plants can be regenerated, plant calli, plant clumps and plant cells that are intact in plants or parts of plants, such as seeds, kernels, cobs, flowers, cotyledons, leaves, stems, buds, roots, root tips, stover, and the like. Plant parts may include processed plant parts or derivatives, including flower, oils, extracts etc. “Parts of a plant” are e.g. shoot vegetative organs/structures, e.g., leaves, stems and tubers; roots, flowers and floral organs/structures, e.g. bracts, sepals, petals, stamens, carpels, anthers and ovules; seed, including embryo, endosperm, and seed coat; fruit and the mature ovary; plant tissue, e.g. vascular tissue, ground tissue, and the like; and cells, e.g. guard cells, egg cells, pollen, trichomes and the like; and progeny of the same. Parts of plants may be attached to or separate from a whole intact plant. Such parts of a plant include, but are not limited to, organs, tissues, and cells of a plant, and preferably seeds. A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. “Plant cell culture” means cultures of plant units such as, for example, protoplasts, cell culture cells, cells in plant tissues, pollen, pollen tubes, ovules, embryo sacs, zygotes and embryos at various stages of development. “Plant material” refers to leaves, stems, roots, flowers or flower parts, fruits, pollen, egg cells, zygotes, seeds, cuttings, cell or tissue cultures, or any other part or product of a plant, including meal. This also includes callus or callus tissue as well as extracts (such as extracts from taproots) or samples. A “plant organ” is a distinct and visibly structured and differentiated part of a plant such as a root, stem, leaf, flower bud, or embryo. “Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In certain embodiments, the plant part or derivative is or comprises (functional) propagation material, such as germplasm, a seed, or plant embryo or other material from which a plant can be regenerated. In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, etc.

As used herein, the terms “progeny” and “progeny plant” refer to a plant generated from sexual reproduction from one or more parent plants. A progeny plant can be obtained by selfing a single parent plant, or by crossing two parental plants. For instance, a progeny plant can be obtained by selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1 s, F2 s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids. The term “progeny” can in certain embodiments be used interchangeably with “offspring”, in particular when the plant or plant material is derived from sexual crossing of parent plants. According to the present invention, progeny preferably refers to the F1 progeny.

As used herein, the terms “crossed” or “cross” or “crossing” means the fusion of gametes via pollination to produce progeny (i.e., cells, seeds, or plants). The term encompasses both sexual crosses (the pollination of one plant by another) and self-fertilization (selfing, self-pollination, i.e., when the pollen and ovule (or microspores and megaspores) are from the same plant or genetically identical plants). Preferably, crossing as referred to herein fertilization of one plant by another plant, i.e. not self-pollination.

As used herein, the term plant population may be used interchangeably with population of plants. A plant population preferably comprises a multitude of individual plants, such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000.

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In the case of haploid induction use of color markers, such as R Navajo, and other markers including transgenes visualized by the presences or absences of color within the seed evidence if the seed is an induced haploid seed. The use of R Navajo as a color marker and the use of transgenes is well known in the art as means to detect induction of haploid seed on the female plant. In other cases, a phenotype is the result of interactions among several genes, which in some embodiments also results from an interaction of the plant and/or plant cell with its environment.

As used herein, “maize” refers to a plant of the species Zea mays, preferably Zea mays ssp mays.

As used herein, the term “male sterile” plant (line, cultivar, or variety) has its ordinary meaning in the art. By means of further guidance, and without limitation, the term refers to a plant which is unable to produce offspring as a pollen donor, and may result from the failure to produce (functional) anthers, pollen, or gametes. Cytoplasmic male sterile plants have cytoplasmic genes, usually in the mitochondria, that encode factors that disrupt or prevent pollen development, making them male-sterile, with male sterility inherited maternally. The utilization of cytoplasmic male sterility for hybrid seed production typically requires three separate plant lines: the male-sterile line, an isogeneic male-fertile line for propagation (“maintainer line”) and a line for restoring fertility to the hybrid so that it can produce seed (“restorer line”). The male-sterile line is used as the receptive parent in a hybrid cross, the maintainer line is genetically identical to the male-sterile line, excepting that it lacks the cytoplasmic sterility factors, and the restorer line is any line that masks the cytoplasmic sterility factor. The restorer line is very important for those plants, such as grain sorghum or cotton, the useful crop of which is the seed itself or seed-associated structures. Genetic male sterility is similar to cytoplasmic male sterility, but differs in that the sterility factors are encoded in nuclear DNA. Typically, genetic male sterility refers to a change in a plant's genetic structure which results in its ability to produce and/or spread viable pollen. Genetic male sterile plant lines may occur naturally. It is also possible to create a male-sterile plant line using recombinant techniques. Whether naturally occurring or transgenic, male-sterile lines still require the use of a sister maintainer line for their propagation, which of necessity leads to a minimum of 50% male-fertile plants in propagated seed. This is a result of the genetics of male-sterility and maintainer lines. If the male-sterility factor is recessive, as most are, a male-sterile plant would have to be homozygous recessive in order to display the trait. Preferably, according to the invention male sterility refers to genetical male sterility. Preferably, according to the invention male sterility is not or does not encompass cytoplasmic male sterility.

Preferably, according to the invention CMS as referred to herein is CMS-C (or C-type CMS), although other types of CMS are also envisaged, including CMS-T and CMS-S.

As used herein, the term “restorer” or “restorer of fertility” means the gene(s) that restore(s) fertility to a CMS plant. The term “restorer” may also mean the plant or line carrying the restorer gene. By means of extension, the term restorer can be applied to a restorer locus (allele), haplotype, or genotype, meaning a locus (allele), haplotype, or genotype carrying the restores gene or being responsible for the restorer phenotype. According to the invention, the restorer gene, locus (allele), haplotype, genotype, or phenotype is associated/linked with the polymorphisms (alleles), polynucleic acids, or markers of the invention as described herein elsewhere. Accordingly, a restorer locus (allele) or fertility restorer locus (allele) refers to a genomic interval carrying the restorer gene(s), and is characterized by the presence of one or more of the polymorphisms (alleles), polynucleic acids, or molecular markers as described herein. According to the invention, the restorer is not (solely) or does not (solely) comprise Rf4.

The combination of any one or more of the marker(s) (allele(s)) of the invention may be referred to as a marker haplotype of the invention.

As used herein, the term “maintainer” may equally be used for the male fertile as well as the (isogenic) male sterile lines, and hence refers to a plant (or line) which does not have the restorer phenotype and/or comprise the restorer genotype, haplotype, or locus (allele) (all either heterozygous or homozygous), as opposed to the term “restorer”, which does have the restorer phenotype and/or comprises the restorer genotype, haplotype, or (allele) (all either heterozygous or homozygous), preferably the restorer phenotype, genotype, haplotype or locus (allele) of the present invention. Accordingly, the term “maintainer” may be used equally for the maintainer line sensu strictu, i.e. the isogenic fertile counterpart of the CMS line for use in “maintaining” the CMS line, as well as for the CMS line itself. In certain embodiments, the maintainer does not have the restorer phenotype and/or comprise the restorer genotype, haplotype, or locus (allele) of the invention, such as any one or more molecular markers (alleles) of the invention, in particular the molecular markers (alleles) associated/linked with the restorer phenotype, genotype, haplotype, or locus (allele) of the invention, which may be homozygous or heterozygous. In certain embodiments, the maintainer has a different restorer phenotype and/or comprise the restorer genotype, haplotype, or locus (allele) than the restorer phenotype and/or comprise the restorer genotype, haplotype, or locus (allele) of the invention, e.g. Rf4, which may be homozygous or heterozygous.

The term “locus” (loci plural) means a specific place or places or a site on a chromosome where for example a QTL/haplotype, a gene or genetic marker is found. As used herein, the term “quantitative trait locus” or “QTL” has its ordinary meaning known in the art. By means of further guidance, and without limitation, a QTL may refer to a region of DNA that is associated with the differential expression of a quantitative phenotypic trait in at least one genetic background, e.g., in at least one breeding population. The region of the QTL encompasses or is closely linked to the gene or genes that affect the trait in question.

As used herein, the term “allele” or “alleles” refers to one or more alternative forms, i.e. different nucleotide sequences, of a locus.

An “allele of a locus” can comprise multiple genes or other genetic factors within a contiguous genomic region or linkage group, such as a haplotype. An allele of a locus can denote a haplotype within a specified window wherein said window is a contiguous genomic region that can be defined, and tracked, with a set of one or more polymorphic markers. A haplotype can be defined by the unique fingerprint of alleles at each marker within the specified window. A locus may encode for one or more alleles that affect the expressivity of a continuously distributed (quantitative) phenotype. In certain embodiments, the locus, allele, polynucleic acid, or molecular marker (allele) as described herein may be homozygous. In certain embodiments, the locus, allele, polynucleic acid, or molecular marker (allele) as described herein may be heterozygous.

As used herein, the term “mutant alleles” or “mutation” of alleles include alleles having one or more mutations, such as insertions, deletions, stop codons, base changes (e.g., transitions or transversions), or alterations in splice junctions, which may or may not give rise to altered gene products. Modifications in alleles may arise in coding or non-coding regions (e.g. promoter regions, exons, introns or splice junctions).

A “marker” is a (means of finding a position on a) genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. The term marker locus is the locus (gene, sequence or nucleotide) that the marker detects. “Marker” or “molecular marker” or “marker locus” may also be used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population. With regard to a SNP marker, allele refers to the specific nucleotide base present at that SNP locus in that individual plant.

“Fine-mapping” refers to methods by which the position of a genomic region (e.g. QTL) can be determined more accurately (narrowed down) and by which the size of the introgression fragment comprising the QTL is reduced. For example Near Isogenic Lines for the QTL or haplotype (QTL/haplotype-NILs) can be made, which contain different, overlapping fragments of the introgression fragment within an otherwise uniform genetic background of the recurrent parent. Such lines can then be used to map on which fragment the QTL/haplotype is located and to identify a line having a shorter introgression fragment comprising the QTL/haplotype.

“Marker assisted selection” (of MAS) is a process by which individual plants are selected based on marker genotypes. “Marker assisted counter-selection” is a process by which marker genotypes are used to identify plants that will not be selected, allowing them to be removed from a breeding program or planting. Marker assisted selection uses the presence of molecular markers, which are genetically linked to a particular locus or to a particular chromosome region (e.g. introgression fragment, transgene, polymorphism, mutation, etc), to select plants for the presence of the specific locus or region (introgression fragment, transgene, polymorphism, mutation, etc). For example, a molecular marker genetically linked to a genomic region (e.g. haplotype) or gene (e.g. the RLK1 allele conferring pathogen resistance) as defined herein, can be used to detect and/or select plants comprising the HT2/HT3 on chromosome 8. The closer the genetic linkage of the molecular marker to the locus (e.g. about 7 cM, 6 cM, 5 cM, 4 cM, 3 cM, 2 cM, 1 cM, 0.5 cM or less), the less likely it is that the marker is dissociated from the locus through meiotic recombination. Likewise, the closer two markers are linked to each other (e.g. within 7 or 5 cM, 4 cM, 3 cM, 2 cM, 1 cM or less) the less likely it is that the two markers will be separated from one another (and the more likely they will co-segregate as a unit). A marker “within 7 cM or within 5 cM, 3 cM, 2 cM, or 1 cM” of another marker refers to a marker which genetically maps to within the 7 cM or 5 cM, 3 cM, 2 cM, or 1 cM region flanking the marker (i.e. either side of the marker). Similarly, a marker within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of another marker refers to a marker which is physically located within the 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less, of the genomic DNA region flanking the marker (i.e. either side of the marker). “LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favour the presence of linkage and a LOD score greater than 3.0 is considered evidence for linkage. A LOD score of +3 indicates 1000 to 1 odds that the linkage being observed did not occur by chance.

A “marker haplotype” refers to a combination of (marker) alleles at a (marker) locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

“Genetic markers” are nucleic acids that are polymorphic in a population and where the alleles of which can be detected and distinguished by one or more analytic methods, e.g., RFLP, AFLP, isozyme, SNP, SSR, and the like. The terms “molecular marker” and “genetic marker” are used interchangeably herein. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

As referred to herein, a polynucleic acid of the invention as described herein, is said to be flanked by certain molecular markers or molecular marker alleles if the polynucleic acid is comprised within a polynucleic acid wherein respectively a first marker (allele) is located upstream (i.e. 5′) of said polynucleic acid and a second marker (allele) is located downstream (i.e. 3′) of said polynucleic acid. Such first and second marker (allele) may border the polynucleic acid. The nucleic acid may equally comprise such first and second marker (allele), such as respectively at or near the 5′ and 3′ end, for instance respectively within 50 kb of the 5′ and 3′ end, preferably within 10 kb of the 5′ and 3′ end, such as within 5 kb of the 5′ and 3′ end, within 1 kb of the 5′ and 3′ end, or less.

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”. The term “indel” refers to an insertion or deletion, wherein one line may be referred to as having an inserted nucleotide or piece of DNA relative to a second line, or the second line may be referred to as having a deleted nucleotide or piece of DNA relative to the first line.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

An allele “negatively” correlates with a trait when it is linked to it and when presence of the allele is an indicator that a desired trait or trait form will not occur in a plant comprising the allele. An allele “positively” correlates with a trait when it is linked to it and when presence of the allele is an indicator that the desired trait or trait form will occur in a plant comprising the allele.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to gray leaf spot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosomal segment are also “genetically linked”, typically within a genetic recombination distance of less than or equal to 50 cM, e.g., about 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.75, 0.5, 0.25 cM or less. That is, two genetic elements within a single chromosomal segment undergo recombination during meiosis with each other at a frequency of less than or equal to about 50%, e.g., about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25% or less. “Closely linked” markers display a cross over frequency with a given marker of about 10% or less, e.g., 9%, 8%, 7%, 6%, 5%, 4%, 3%, 2%, 1%, 0.75%, 0.5%, 0.25% or less (the given marker locus is within about 10 cM of a closely linked marker locus, e.g., 9, 8, 7, 6, 5, 4, 3, 2, 1, 0.75, 0.5, 0.25 cM or less of a closely linked marker locus). Put another way, closely linked marker loci co-segregate at least about 90% the time, e.g., 91%, 92%, 93%, 94%, 95%, 96%, 97%, 98%, 99%, 99.5%, 99.75%, or more of the time.

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, a haplotype, a QTL, a transgene, or the like. In any case, offspring comprising the desired allele can be repeatedly backcrossed to a line having a desired genetic background and selected for the desired allele, to result in the allele becoming fixed in a selected genetic background. The process of “introgressing” is often referred to as “backcrossing” when the process is repeated two or more times. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

A genetic element, an introgression fragment, or a gene or allele conferring a trait is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only pants comprising the genetic element, locus, introgression fragment, gene or allele can be used, but also progeny/descendants from such plants which have been selected to retain the genetic element, locus, introgression fragment, gene or allele, can be used and are encompassed herein. Whether a plant (or genomic DNA, cell or tissue of a plant) comprises the same genetic element, locus, introgression fragment, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic plants may also be encompassed.

In certain embodiments, the polynucleic acid is introduced (and genomically integrated) recombinantly or transgenically. The polynucleic acid may be introduced (and genomically integrated) at the native locus, to replace an endogenous polynucleic acid (such as the polynucleic acid not conferring pathogen resistance), or may be introduced (and genomically integrated) at a locus different than the endogenous locus (e.g. by random integration in the genome). In certain embodiments, the method for generating a maize plant or plant part comprises transforming a plant or plant part, preferably a plant cell, more preferably a protoplast, with the polynucleic acid, which may be provided on a vector, as described herein elsewhere. In certain embodiments, the polynucleic acid has a different sequence than an endogenous polynucleic acid (such as an endogenous polynucleic acid not conferring pathogen resistance).

As used herein the terms “genetic engineering”, “transformation” and “genetic modification” are all used herein as synonyms for the transfer of isolated and cloned genes into the DNA, usually the chromosomal DNA or genome, of another organism.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). The term “transgenic” here means genetically modified by the introduction of a non-endogenous nucleic acid sequence. Typically a species-specific nucleic acid sequence is introduced in a form, arrangement or quantity into the cell in a location where the nucleic acid sequence does not occur naturally in the cell. This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Gene editing” or “genome editing” refers to genetic engineering in which in which DNA or RNA is inserted, deleted, modified or replaced in the genome of a living organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas9) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically introduce one or more of the molecular marker (allele) according to the invention as described herein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As uses herein, “hybridization” or “hybridizing” refers to a reaction in which one or more polynucleotides react to form a complex that is stabilized via hydrogen bonding between the bases of the nucleotide residues, i.e. a process in which a single-stranded nucleic acid molecule attaches itself to a complementary nucleic acid strand, i.e. agrees with this base pairing. Standard procedures for hybridization are described, for example, in Sambrook et al. (Molecular Cloning. A Laboratory Manual, Cold Spring Harbor Laboratory Press, 3rd edition 2001). The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence. Preferably this will be understood to mean an at least 50%, more preferably at least 55%, 60%, 65%, 70%, 75%, 80% or 85%, more preferably 90%, 91%, 92%, 93%, 94%, 95%, 96%, 97%, 98% or 99% of the bases of the nucleic acid strand form base pairs with the complementary nucleic acid strand. The possibility of such binding depends on the stringency of the hybridization conditions.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance a genetic element may be replaced by gene editing in which a recombination template is provided. The DNA may be cut upstream and downstream of a sequence which needs to be replaced. As such, the sequence to be replaced is excised from the DNA. Through HDR, the excised sequence is then replaced by the template. In certain embodiments, the marker (allele) of the invention as described herein may be provided on/as a template. By designing the system such that double strand breaks are introduced upstream and downstream of the corresponding region in the genome of a plant not comprising the marker (allele), this region is excised and can be replaced with the template comprising the marker (allele) of the invention. In this way, introduction of the marker (allele) of the invention in a plant need not involve multiple backcrossing, in particular in a plant of specific genetic background. Similarly, the polynucleic acid of the invention may be provided on/as a template. More advantageously however, the polynucleic acid of the invention may be generated without the use of a recombination template, but solely through the endonuclease action leading to a double strand DNA break which is repaired by NHEJ, resulting in the generation of indels.

In certain embodiments, the nucleic acid modification is effected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nucleases. The products are then separated by size, such as by HPLC. See also McCallum et al. “Targeted screening for induced mutations”; Nat Biotechnol. 2000 April; 18(4):455-7 and McCallum et al. “Targeting induced local lesions IN genomes (TILLING) for plant functional genomics”; Plant Physiol. 2000 June; 123(2):439-42.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. As used herein, the term “heterozygote” refers to an individual cell or plant having different alleles at one or more or all loci. When the term is used with reference to a specific locus or gene, it means at least that locus or gene has different alleles. As used herein, the term “heterozygous” means a genetic condition existing when different alleles reside at corresponding loci on homologous chromosomes. In certain embodiments, the haplotype and/or one or more marker(s) as described herein is/are homozygous. In certain embodiments, the haplotype and/or one or more marker(s) as described herein are heterozygous. In certain embodiments, the haplotype allele and/or one or more marker(s) allele(s) as described herein is/are homozygous. In certain embodiments, the haplotype allele and/or one or more marker(s) allele(s) as described herein are heterozygous.

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the B12seq program can be found in the readme file accompanying BLASTZ. B12seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq I .txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. The following command will generate an output file containing a comparison between two sequences: C:\B12seq -i c:\seql .txt -j c:\seq2.txt -p blastn -o c:\output.txt -q - 1 -r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the B12seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used. The terms “nucleotide sequence(s)”, “polynucleotide(s)”, “nucleic acid sequence(s)”, “nucleic acid(s)”, “nucleic acid molecule”, “polynucleic acid(s)” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

When reference is made to a nucleic acid sequence (e.g. DNA or genomic DNA) having “substantial sequence identity to” a reference sequence or having a sequence identity of at least 80%>, e.g. at least 85%, 90%, 95%, 98%> or 99%> nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The term polypeptide also refers to, and does not exclude, modifications of the polypeptide, e.g., glycosylation, acetylation, phosphorylation and the like. Such modifications are well described in basic texts and in more detailed monographs, as well as in the research literature.

Amino acid substitutions encompass amino acid alterations in which an amino acid is replaced with a different naturally-occurring amino acid residue. Such substitutions may be classified as “conservative<1>, in which an amino acid residue contained in the wild-type protein is replaced with another naturally-occurring amino acid of similar character, for example Gly<->Ala, Val<->Ile<->Leu, Asp<->Glu, Lys<->Arg, Asn<->Gln or Phe<->Trp<->Tyr. Substitutions encompassed by the present invention may also be “non-conservative”, in which an amino acid residue which is present in the wild-type protein is substituted with an amino acid with different properties, such as a naturally-occurring amino acid from a different group (e.g. substituting a charged or hydrophobic amino acid with alanine. “Similar amino acids”, as used herein, refers to amino acids that have similar amino acid side chains, i.e. amino acids that have polar, non-polar or practically neutral side chains. “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the center of the protein where they can interact with similar non-polar neighbours (“hydrophobic” amino acids”). Examples of amino acids that have polar side chains are arginine, asparagine, aspartate, cysteine, glutamine, glutamate, histidine, lysine, serine, and threonine (all hydrophilic, except for cysteine which is hydrophobic). Examples of amino acids that have non-polar side chains are alanine, glycine, isoleucine, leucine, methionine, phenylalanine, proline, and tryptophan (all hydrophobic, except for glycine which is neutral).

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5-terminus and a translation stop codon at the 3′-terminus. A coding sequence can include, but is not limited to mRNA, cDNA, recombinant nucleic acid sequences or genomic DNA, while introns may be present as well under certain circumstances.

A used herein, the term “endogenous” refers to a gene or allele which is present in its natural genomic location. The term “endogenous” can be used interchangeably with “native”. This does not however exclude the presence of one or more nucleic acid differences with the wild-type allele. In particular embodiments, the difference with a wild-type allele can be limited to less than 9 preferably less than 6, more particularly less than 3 nucleotide differences, such as 0 nucleotides difference. More particularly, the difference with the wildtype sequence can be in only one nucleotide. Preferably, the endogenous allele encodes a modified protein having less than 9, preferably less than 6, more particularly less than 3 and even more preferably only one or no amino acid difference with the wild-type protein.

A used herein, the term “exogenous polynucleotide” refers to a polynucleotide, such as a gene (or cDNA) or allele which is or has been recombinantly introduced in a cell (or plant). The exogenous polynucleotide may be episomal or genomically integrated. Integration may be random or site-directed. Integration may include replacement of a corresponding endogenous polynucleotide. It will be understood that an exogenous polynucleotide is not naturally present in the cell or plant.

As used herein, the B73 reference genome AGPv4 (or AGPv04) refers to the assembly B73 RefGen_v4 (also known as AGPv4, B73 RefGen_v4) as provided on the Maize Genetics and Genomics Database (https://www.maizegdb.org/genome/genome_assembly/Zm-B73-REFERENCE-GRAMENE-4.0).

Methods for screening for the presence of the polynucleic acid of the invention, or the (molecular) marker(s) (alleles) as described herein are known in the art. Without limitation, screening may encompass or comprise sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay), etc.

In an aspect, the invention relates to a method for identifying a plant, plant part or plant material, in particular a maize plant or plant part, comprising screening for the presence of or detecting or identifying a cytoplasmic male sterility restorer locus on chromosome 3, in particular RF-03-01, as described herein elsewhere.

In an aspect, the invention relates to a method for identifying a plant, plant part or plant material, in particular a maize plant or plant part, comprising screening for the absence of a cytoplasmic male sterility restorer locus on chromosome 3, in particular RF-03-01, as described herein elsewhere.

In an aspect, the invention relates to a method for identifying a plant, plant part or plant material, in particular a maize plant or plant part, comprising screening for the presence of or detecting or identifying a cytoplasmic male sterility maintainer locus on chromosome 3, in particular RF-03-01, as described herein elsewhere.

In an aspect, the invention relates to a method for identifying a plant, plant part or plant material, in particular a maize plant or plant part, comprising screening for the absence of a cytoplasmic male sterility maintainer locus on chromosome 3, in particular RF-03-01, as described herein elsewhere.

In an aspect, the invention relates to a method for identifying a plant, plant part or plant material, in particular a maize plant or plant part, comprising screening for the presence of or detecting or identifying (a haplotype associated with/linked) with a cytoplasmic male sterility restorer locus on chromosome 3, in particular RF-03-01, as described herein elsewhere.

In an aspect, the invention relates to a method for identifying a plant or plant part, in particular a maize plant or plant part, comprising screening for the absence of (a haplotype associated with/linked with) a cytoplasmic male sterility restorer locus on chromosome 3, in particular RF-03-01, as described herein elsewhere.

In an aspect, the invention relates to a method for identifying a plant, plant part or plant material, in particular a maize plant or plant part, comprising screening for the presence of or detecting or identifying (a haplotype associated with/linked with) a cytoplasmic male sterility maintainer locus on chromosome 3, in particular RF-03-01, as described herein elsewhere.

In an aspect, the invention relates to a method for identifying a plant, plant part or plant material, in particular a maize plant or plant part, comprising screening for the absence of (a haplotype associated with/linked with) a cytoplasmic male sterility maintainer locus on chromosome 3, in particular RF-03-01, as described herein elsewhere.

In an aspect, the invention relates to a method for identifying a plant, plant part or plant material, in particular a maize plant or plant part, comprising screening for the presence of or detecting or identifying one or more molecular markers of Table 4 or Table 5. In an aspect, the invention relates to a method for identifying a plant or plant part, in particular a maize plant or plant part, comprising screening for the presence of or detecting or identifying two or more molecular markers of Table 4 or Table 5. In an aspect, the invention relates to a method for identifying a plant or plant part, in particular a maize plant or plant part, comprising screening for the presence of or detecting or identifying three or more molecular markers of Table 4 or Table 5. In certain embodiments, the method is a method for identifying a plant or plant part comprising a cytoplasmic male sterility restorer locus. In certain embodiments, the method is a method for identifying a plant or plant part comprising a cytoplasmic male sterility maintainer locus.

In an aspect, the invention relates to a method for identifying a plant, plant part or plant material, in particular a maize plant or plant part, comprising screening for the presence of or detecting or identifying one or more molecular marker alleles of Table 4 or Table 5 (and having a polymorphism corresponding to or comprised in a cytoplasmic male sterility restorer locus). In an aspect, the invention relates to a method for identifying a plant or plant part, in particular a maize plant or plant part, comprising screening for the presence of or detecting or identifying two or more molecular marker alleles of Table 4 or Table 5 (and having a polymorphism corresponding to or comprised in a cytoplasmic male sterility restorer locus). In an aspect, the invention relates to a method for identifying a plant or plant part, in particular a maize plant or plant part, comprising screening for the presence of or detecting or identifying three or more molecular marker alleles of Table 4 or Table 5 (and having a polymorphism corresponding to or comprised in a cytoplasmic male sterility restorer locus).

In an aspect, the invention relates to a method for identifying a plant, plant part or plant material, in particular a maize plant or plant part, comprising screening for the presence of or detecting or identifying one or more molecular marker alleles of Table 4 or Table 5 (and having a polymorphism corresponding to or comprised in a cytoplasmic male sterility maintainer locus). In an aspect, the invention relates to a method for identifying a plant or plant part, in particular a maize plant or plant part, comprising screening for the presence of or detecting or identifying two or more molecular marker alleles of Table 4 or Table 5 (and having a polymorphism corresponding to or comprised in a cytoplasmic male sterility maintainer locus). In an aspect, the invention relates to a method for identifying a plant or plant part, in particular a maize plant or plant part, comprising screening for the presence of or detecting or identifying three or more molecular marker alleles of Table 4 or Table 5 (and having a polymorphism corresponding to or comprised in a cytoplasmic male sterility maintainer locus).

In an aspect, the invention relates to a method for identifying a plant, plant part or plant material, in particular a maize plant or plant part, comprising screening for the presence of or detecting or identifying one or more polynucleic acid sequences having a sequence as set forth in any of SEQ ID NOs: 1, 5, 9, 13, 2, 6, 10, or 14, or having a sequence which is at least 80%, preferably at least 85%, more preferably at least 90%, most preferably at least 95% identical to a sequence as set forth in any one or more of SEQ ID NOs: 1, 5, 9, 13, 2, 6, 10, or 14. In an aspect, the invention relates to a method for identifying a plant or plant part, in particular a maize plant or plant part, comprising screening for the presence of or detecting or identifying two or more polynucleic acid sequences having a sequence as set forth in any of SEQ ID NOs: 1, 5, 9, 13, 2, 6, 10, or 14, or having a sequence which is at least 80%, preferably at least 85%, more preferably at least 90%, most preferably at least 95% identical to a sequence as set forth in any one or more of SEQ ID NOs: 1, 5, 9, 13, 2, 6, 10, or 14. In an aspect, the invention relates to a method for identifying a plant or plant part, in particular a maize plant or plant part, comprising screening for the presence of or detecting or identifying three or more polynucleic acid sequences having a sequence as set forth in any of SEQ ID NOs: 1, 5, 9, 13, 2, 6, 10, or 14, or having a sequence which is at least 80%, preferably at least 85%, more preferably at least 90%, most preferably at least 95% identical to a sequence as set forth in any one or more of SEQ ID NOs: 1, 5, 9, 13, 2, 6, 10, or 14. In certain embodiments, the method is a method for identifying a plant or plant part comprising a cytoplasmic male sterility restorer locus. In certain embodiments, the method is a method for identifying a plant or plant part comprising a cytoplasmic male sterility maintainer locus.

In an aspect, the invention relates to a method for identifying a plant, plant part or plant material, in particular a maize plant or plant part, comprising screening for the presence of or detecting or identifying one or more polynucleic acid sequences having a sequence as set forth in any of SEQ ID NOs: 1, 5, 9, or 13, or having a sequence which is at least 80%, preferably at least 85%, more preferably at least 90%, most preferably at least 95% identical to a sequence as set forth in any one or more of SEQ ID NOs: 1, 5, 9, or 13. In an aspect, the invention relates to a method for identifying a plant or plant part, in particular a maize plant or plant part, comprising screening for the presence of or detecting or identifying two or more polynucleic acid sequences having a sequence as set forth in any of SEQ ID NOs: 1, 5, 9, or 13, or having a sequence which is at least 80%, preferably at least 85%, more preferably at least 90%, most preferably at least 95% identical to a sequence as set forth in any one or more of SEQ ID NOs: 1, 5, 9, or 13. In an aspect, the invention relates to a method for identifying a plant or plant part, in particular a maize plant or plant part, comprising screening for the presence of or detecting or identifying three or more polynucleic acid sequences having a sequence as set forth in any of SEQ ID NOs: 1, 5, 9, or 13, or having a sequence which is at least 80%, preferably at least 85%, more preferably at least 90%, most preferably at least 95% identical to a sequence as set forth in any one or more of SEQ ID NOs: 1, 5, 9, or 13. In certain embodiments, the method is a method for identifying a plant or plant part comprising a cytoplasmic male sterility restorer locus.

In an aspect, the invention relates to a method for identifying a plant, plant part or plant material, in particular a maize plant or plant part, comprising screening for the presence of or detecting or identifying one or more polynucleic acid sequences having a sequence as set forth in any of SEQ ID NOs: 2, 6, 10, or 14, or having a sequence which is at least 80%, preferably at least 85%, more preferably at least 90%, most preferably at least 95% identical to a sequence as set forth in any one or more of SEQ ID NOs: 2, 6, 10, or 14. In an aspect, the invention relates to a method for identifying a plant or plant part, in particular a maize plant or plant part, comprising screening for the presence of or detecting or identifying two or more polynucleic acid sequences having a sequence as set forth in any of SEQ ID NOs: 2, 6, 10, or 14, or having a sequence which is at least 80%, preferably at least 85%, more preferably at least 90%, most preferably at least 95% identical to a sequence as set forth in any one or more of SEQ ID NOs: 2, 6, 10, or 14. In an aspect, the invention relates to a method for identifying a plant or plant part, in particular a maize plant or plant part, comprising screening for the presence of or detecting or identifying three or more polynucleic acid sequences having a sequence as set forth in any of SEQ ID NOs: 2, 6, 10, or 14, or having a sequence which is at least 80%, preferably at least 85%, more preferably at least 90%, most preferably at least 95% identical to a sequence as set forth in any one or more of SEQ ID NOs: 2, 6, 10, or 14. In certain embodiments, the method is a method for identifying a plant or plant part comprising a cytoplasmic male sterility maintainer locus.

In an aspect, the invention relates to a method for identifying a plant, plant part or plant material, in particular a maize plant or plant part, comprising screening for the presence of or detecting or identifying one or more genes selected from Zm00001d043358, Zm00001d043352, Zm00001d043356, and Zm00001d043357 having respectively a coding sequence as set forth in any of SEQ ID NOs: 201, 203, 205, 207, 3, 7, 11, or 15, or having a coding sequence which is at least 80%, preferably at least 85%, more preferably at least 90%, most preferably at least 95% identical to a sequence as set forth in any one or more of SEQ ID NOs: 201, 203, 205, 207, 3, 7, 11, or 15. In an aspect, the invention relates to a method for identifying a plant or plant part, in particular a maize plant or plant part, comprising screening for the presence of or detecting or identifying two or more genes selected from Zm00001d043358, Zm00001d043352, Zm00001d043356, and Zm00001d043357 having respectively a coding sequence as set forth in any of SEQ ID NOs: 201, 203, 205, 207, 3, 7, 11, or 15, or having a coding sequence which is at least 80%, preferably at least 85%, more preferably at least 90%, most preferably at least 95% identical to a sequence as set forth in any one or more of SEQ ID NOs: 201, 203, 205, 207, 3, 7, 11, or 15. In an aspect, the invention relates to a method for identifying a plant or plant part, in particular a maize plant or plant part, comprising screening for the presence of or detecting or identifying three or more genes selected from Zm00001d043358, Zm00001d043352, Zm00001d043356, and Zm00001d043357 having respectively a coding sequence as set forth in any of SEQ ID NOs: 201, 203, 205, 207, 3, 7, 11, or 15, or having a coding sequence which is at least 80%, preferably at least 85%, more preferably at least 90%, most preferably at least 95% identical to a sequence as set forth in any one or more of SEQ ID NOs: 201, 203, 205, 207, 3, 7, 11, or 15. In certain embodiments, the method is a method for identifying a plant or plant part comprising a cytoplasmic male sterility restorer locus. In certain embodiments, the method is a method for identifying a plant or plant part comprising a cytoplasmic male sterility maintainer locus.

In an aspect, the invention relates to a method for identifying a plant, plant part or plant material, in particular a maize plant or plant part, comprising screening for the presence of or detecting or identifying one or more genes selected from Zm00001d043358, Zm00001d043352, Zm00001d043356, and Zm00001d043357 having respectively a coding sequence as set forth in any of SEQ ID NOs: 201, 203, 205, or 207, or having a coding sequence which is at least 80%, preferably at least 85%, more preferably at least 90%, most preferably at least 95% identical to a sequence as set forth in any one or more of SEQ ID NOs: 201, 203, 205, or 207. In an aspect, the invention relates to a method for identifying a plant or plant part, in particular a maize plant or plant part, comprising screening for the presence of or detecting or identifying two or more genes selected from Zm00001d043358, Zm00001d043352, Zm00001d043356, and Zm00001d043357 having respectively a coding sequence as set forth in any of SEQ ID NOs: 201, 203, 205, or 207, or having a coding sequence which is at least 80%, preferably at least 85%, more preferably at least 90%, most preferably at least 95% identical to a sequence as set forth in any one or more of SEQ ID NOs: 201, 203, 205, or 207. In an aspect, the invention relates to a method for identifying a plant or plant part, in particular a maize plant or plant part, comprising screening for the presence of or detecting or identifying three or more genes selected from Zm00001d043358, Zm00001d043352, Zm00001d043356, and Zm00001d043357 having respectively a coding sequence as set forth in any of SEQ ID NOs: 201, 203, 205, or 207, or having a coding sequence which is at least 80%, preferably at least 85%, more preferably at least 90%, most preferably at least 95% identical to a sequence as set forth in any one or more of SEQ ID NOs: 201, 203, 205, or 207. In certain embodiments, the method is a method for identifying a plant or plant part comprising a cytoplasmic male sterility restorer locus.

In an aspect, the invention relates to a method for identifying a plant, plant part or plant material, in particular a maize plant or plant part, comprising screening for the presence of or detecting or identifying one or more genes selected from Zm00001d043358, Zm00001d043352, Zm00001d043356, and Zm00001d043357 having respectively a coding sequence as set forth in any of SEQ ID NOs: 3, 7, 11, or 15, or having a coding sequence which is at least 80%, preferably at least 85%, more preferably at least 90%, most preferably at least 95% identical to a sequence as set forth in any one or more of SEQ ID NOs: 3, 7, 11, or 15. In an aspect, the invention relates to a method for identifying a plant or plant part, in particular a maize plant or plant part, comprising screening for the presence of or detecting or identifying two or more genes selected from Zm00001d043358, Zm00001d043352, Zm00001d043356, and Zm00001d043357 having respectively a coding sequence as set forth in any of SEQ ID NOs: 3, 7, 11, or 15, or having a coding sequence which is at least 80%, preferably at least 85%, more preferably at least 90%, most preferably at least 95% identical to a sequence as set forth in any one or more of SEQ ID NOs: 3, 7, 11, or 15. In an aspect, the invention relates to a method for identifying a plant or plant part, in particular a maize plant or plant part, comprising screening for the presence of or detecting or identifying three or more genes selected from Zm00001d043358, Zm00001d043352, Zm00001d043356, and Zm00001d043357 having respectively a coding sequence as set forth in any of SEQ ID NOs: 3, 7, 11, or 15, or having a coding sequence which is at least 80%, preferably at least 85%, more preferably at least 90%, most preferably at least 95% identical to a sequence as set forth in any one or more of SEQ ID NOs: 3, 7, 11, or 15. In certain embodiments, the method is a method for identifying a plant or plant part comprising a cytoplasmic male sterility maintainer locus.

In an aspect, the invention relates to a method for identifying a plant, plant part or plant material, in particular a maize plant or plant part, comprising screening for the presence of or detecting or identifying one or more genes selected from Zm00001d043358, Zm00001d043352, Zm00001d043356, and Zm00001d043357 encoding respectively a polypeptide having a sequence as set forth in any of SEQ ID NOs: 202, 204, 206, 208, 4, 8, 12, or 16, or having a sequence which is at least 80%, preferably at least 85%, more preferably at least 90%, most preferably at least 95% identical to a sequence as set forth in any one or more of SEQ ID NOs: 202, 204, 206, 208, 4, 8, 12, or 16. In an aspect, the invention relates to a method for identifying a plant or plant part, in particular a maize plant or plant part, comprising screening for the presence of or detecting or identifying two or more genes selected from Zm00001d043358, Zm00001d043352, Zm00001d043356, and Zm00001d043357 encoding respectively a polypeptide having a sequence as set forth in any of SEQ ID NOs: 202, 204, 206, 208, 4, 8, 12, or 16, or having a sequence which is at least 80%, preferably at least 85%, more preferably at least 90%, most preferably at least 95% identical to a sequence as set forth in any one or more of SEQ ID NOs: 202, 204, 206, 208, 4, 8, 12, or 16. In an aspect, the invention relates to a method for identifying a plant or plant part, in particular a maize plant or plant part, comprising screening for the presence of or detecting or identifying three or more genes selected from Zm00001d043358, Zm00001d043352, Zm00001d043356, and Zm00001d043357 encoding respectively a polypeptide having a sequence as set forth in any of SEQ ID NOs: 202, 204, 206, 208, 4, 8, 12, or 16, or having a sequence which is at least 80%, preferably at least 85%, more preferably at least 90%, most preferably at least 95% identical to a sequence as set forth in any one or more of SEQ ID NOs: 202, 204, 206, 208, 4, 8, 12, or 16. In certain embodiments, the method is a method for identifying a plant or plant part comprising a cytoplasmic male sterility restorer locus. In certain embodiments, the method is a method for identifying a plant or plant part comprising a cytoplasmic male sterility maintainer locus.

In an aspect, the invention relates to a method for identifying a plant, plant part or plant material, in particular a maize plant or plant part, comprising screening for the presence of or detecting or identifying one or more genes selected from Zm00001d043358, Zm00001d043352, Zm00001d043356, and Zm00001d043357 encoding respectively a polypeptide having a sequence as set forth in any of SEQ ID NOs: 202, 204, 206, or 208, or having a sequence which is at least 80%, preferably at least 85%, more preferably at least 90%, most preferably at least 95% identical to a sequence as set forth in any one or more of SEQ ID NOs: 202, 204, 206, or 208, preferably selected from Zm00001d043358, Zm00001d043352, and Zm00001d043357 encoding respectively a polypeptide having a sequence as set forth in any of SEQ ID NOs: 202, 204, or 208, or having a sequence which is at least 80%, preferably at least 85%, more preferably at least 90%, most preferably at least 95% identical to a sequence as set forth in any one or more of SEQ ID NOs: 202, 204, or 208. In an aspect, the invention relates to a method for identifying a plant or plant part, in particular a maize plant or plant part, comprising screening for the presence of or detecting or identifying two or more genes selected from Zm00001d043358, Zm00001d043352, Zm00001d043356, and Zm00001d043357 encoding respectively a polypeptide having a sequence as set forth in any of SEQ ID NOs: 202, 204, 206, or 208, or having a sequence which is at least 80%, preferably at least 85%, more preferably at least 90%, most preferably at least 95% identical to a sequence as set forth in any one or more of SEQ ID NOs: 202, 204, 206, or 208, preferably selected from Zm00001d043358, Zm00001d043352, and Zm00001d043357 encoding respectively a polypeptide having a sequence as set forth in any of SEQ ID NOs: 202, 204, or 208, or having a sequence which is at least 80%, preferably at least 85%, more preferably at least 90%, most preferably at least 95% identical to a sequence as set forth in any one or more of SEQ ID NOs: 202, 204, or 208. In an aspect, the invention relates to a method for identifying a plant or plant part, in particular a maize plant or plant part, comprising screening for the presence of or detecting or identifying three or more genes selected from Zm00001d043358, Zm00001d043352, Zm00001d043356, and Zm00001d043357 encoding respectively a polypeptide having a sequence as set forth in any of SEQ ID NOs: 202, 204, 206, or 208, or having a sequence which is at least 80%, preferably at least 85%, more preferably at least 90%, most preferably at least 95% identical to a sequence as set forth in any one or more of SEQ ID NOs: 202, 204, 206, or 208, preferably selected from Zm00001d043358, Zm00001d043352, and Zm00001d043357 encoding respectively a polypeptide having a sequence as set forth in any of SEQ ID NOs: 202, 204, or 208, or having a sequence which is at least 80%, preferably at least 85%, more preferably at least 90%, most preferably at least 95% identical to a sequence as set forth in any one or more of SEQ ID NOs: 202, 204, or 208. In certain embodiments, the method is a method for identifying a plant or plant part comprising a cytoplasmic male sterility restorer locus.

In an aspect, the invention relates to a method for identifying a plant, plant part or plant material, in particular a maize plant or plant part, comprising screening for the presence of or detecting or identifying one or more genes selected from Zm00001d043358, Zm00001d043352, and Zm00001d043357 encoding respectively a polypeptide having a sequence as set forth in any of SEQ ID NOs: 4, 8, or 16, or having a sequence which is at least 80%, preferably at least 85%, more preferably at least 90%, most preferably at least 95% identical to a sequence as set forth in any one or more of SEQ ID NOs: 4, 8, or 16. In an aspect, the invention relates to a method for identifying a plant or plant part, in particular a maize plant or plant part, comprising screening for the presence of or detecting or identifying two or more genes selected from Zm00001d043358, Zm00001d043352, and Zm00001d043357 encoding respectively a polypeptide having a sequence as set forth in any of SEQ ID NOs: 4, 8, or 16, or having a sequence which is at least 80%, preferably at least 85%, more preferably at least 90%, most preferably at least 95% identical to a sequence as set forth in any one or more of SEQ ID NOs: 4, 8, or 16. In an aspect, the invention relates to a method for identifying a plant or plant part, in particular a maize plant or plant part, comprising screening for the presence of or detecting or identifying three or more genes selected from Zm00001d043358, Zm00001d043352, and Zm00001d043357 encoding respectively a polypeptide having a sequence as set forth in any of SEQ ID NOs: 4, 8, or 16, or having a sequence which is at least 80%, preferably at least 85%, more preferably at least 90%, most preferably at least 95% identical to a sequence as set forth in any one or more of SEQ ID NOs: 4, 8, or 16. In certain embodiments, the method is a method for identifying a plant or plant part comprising a cytoplasmic male sterility maintainer locus.

In certain preferred embodiments, when reference is made herein to a sequence which is at least 80%, preferably at least 85%, more preferably at least 90%, most preferably at least 95% identical to a sequence as set forth in a particular SEQ ID NO, such sequence comprises one or more, preferably all, of the polymorphisms (such as SNPs, insertions, or deletions) associated with the maintainer or the restorer locus/allele (and comprised in that SEQ ID NO) as described herein elsewhere, in particular the polymorphisms as described in Table 5. For instance, a sequence which is at least 80%, preferably at least 85%, more preferably at least 90%, most preferably at least 95% identical to a sequence as set forth in SEQ ID NO: 1 may comprise one or more, preferably all restorer (or restorer-associated) polymorphism having identifier 1-39 in Table 5, e.g. a “g” at a position corresponding to position 35 of SEQ ID NO: 1. For instance, a sequence which is at least 80%, preferably at least 85%, more preferably at least 90%, most preferably at least 95% identical to a sequence as set forth in SEQ ID NO: 2 may comprise one or more, preferably all maintainer (or maintainer-associated) polymorphism having identifier 1-39 in Table 5, e.g. a “t” at a position corresponding to position 35 of SEQ ID NO: 2. For instance, a sequence which is at least 80%, preferably at least 85%, more preferably at least 90%, most preferably at least 95% identical to a sequence as set forth in SEQ ID NO: 5 may comprise one or more, preferably all restorer (or restorer-associated) polymorphism having identifier 40-69 in Table 5, e.g. a “a” at a position corresponding to position 486 of SEQ ID NO: 5. For instance, a sequence which is at least 80%, preferably at least 85%, more preferably at least 90%, most preferably at least 95% identical to a sequence as set forth in SEQ ID NO: 6 may comprise one or more, preferably all maintainer (or maintainer-associated) polymorphism having identifier 40-69 in Table 5, e.g. a “t” at a position corresponding to position 486 of SEQ ID NO: 6. For instance, a sequence which is at least 80%, preferably at least 85%, more preferably at least 90%, most preferably at least 95% identical to a sequence as set forth in SEQ ID NO: 9 may comprise one or more, preferably all restorer (or restorer-associated) polymorphism having identifier 70-98 in Table 5, e.g. a “tg” at a position corresponding to position 392-393 of SEQ ID NO: 9. For instance, a sequence which is at least 80%, preferably at least 85%, more preferably at least 90%, most preferably at least 95% identical to a sequence as set forth in SEQ ID NO: 10 may comprise one or more, preferably all maintainer (or maintainer-associated) polymorphism having identifier 70-98 in Table 5, e.g. a “gtggt” at a position corresponding to position 393-397 of SEQ ID NO: 10. For instance, a sequence which is at least 80%, preferably at least 85%, more preferably at least 90%, most preferably at least 95% identical to a sequence as set forth in SEQ ID NO: 13 may comprise one or more, preferably all restorer (or restorer-associated) polymorphism having identifier 99-100 in Table 5, e.g. a “cc” at a position corresponding to position 651-652 of SEQ ID NO: 13. For instance, a sequence which is at least 80%, preferably at least 85%, more preferably at least 90%, most preferably at least 95% identical to a sequence as set forth in SEQ ID NO: 14 may comprise one or more, preferably all maintainer (or maintainer-associated) polymorphism having identifier 99-100 in Table 5, e.g. a “cgc” at a position corresponding to position 652-654 of SEQ ID NO: 14.

While the above SEQ ID NOs are genomic sequences, the skilled person will understand that corresponding polymorphisms in SEQ ID NOs of coding sequences are also implied.

In an aspect, the invention relates to a method for identifying a plant, plant part or plant material, in particular a maize plant or plant part, comprising screening for the presence of or detecting or identifying one or more polynucleic acid sequences having a sequence as set forth in any of SEQ ID NOs: 17 to 200. In an aspect, the invention relates to a method for identifying a plant or plant part, in particular a maize plant or plant part, comprising screening for the presence of or detecting or identifying two or more polynucleic acid sequences having a sequence as set forth in any of SEQ ID NOs: 17 to 200. In an aspect, the invention relates to a method for identifying a plant or plant part, in particular a maize plant or plant part, comprising screening for the presence of or detecting or identifying three or more polynucleic acid sequences having a sequence as set forth in any of SEQ ID NOs: 17 to 200.

In an aspect, the invention relates to a method for identifying a plant, plant part or plant material, in particular a maize plant or plant part, comprising screening for the presence of or detecting or identifying one or more polynucleic acid sequences having a sequence as set forth in any of SEQ ID NOs: 17 to 200 and having a polymorphism corresponding to a cytoplasmic male sterility restorer locus. In an aspect, the invention relates to a method for identifying a plant or plant part, in particular a maize plant or plant part, comprising screening for the presence of or detecting or identifying two or more polynucleic acid sequences having a sequence as set forth in any of SEQ ID NOs: 17 to 200 and having a polymorphism corresponding to a cytoplasmic male sterility restorer locus. In an aspect, the invention relates to a method for identifying a plant or plant part, in particular a maize plant or plant part, comprising screening for the presence of or detecting or identifying three or more polynucleic acid sequences having a sequence as set forth in any of SEQ ID NOs: 17 to 200 and having a polymorphism corresponding to a cytoplasmic male sterility restorer locus.

In an aspect, the invention relates to a method for identifying a plant, plant part or plant material, in particular a maize plant or plant part, comprising screening for the presence of or detecting or identifying one or more polynucleic acid sequences having a sequence as set forth in any of SEQ ID NOs: 17 to 200 and having a polymorphism not corresponding to a cytoplasmic male sterility maintainer locus. In an aspect, the invention relates to a method for identifying a plant or plant part, in particular a maize plant or plant part, comprising screening for the presence of or detecting or identifying two or more polynucleic acid sequences having a sequence as set forth in any of SEQ ID NOs: 17 to 200 and having a polymorphism not corresponding to a cytoplasmic male sterility maintainer locus. In an aspect, the invention relates to a method for identifying a plant or plant part, in particular a maize plant or plant part, comprising screening for the presence of or detecting or identifying three or more polynucleic acid sequences having a sequence as set forth in any of SEQ ID NOs: 17 to 200 and having a polymorphism not corresponding to a cytoplasmic male sterility maintainer locus.

In an aspect, the invention relates to a method for identifying a plant, plant part or plant material, in particular a maize plant or plant part, comprising screening for the presence of or detecting or identifying one or more, two or more, or three or more of the following polymorphisms (positions correspond to maize reference B73 AGPv4 chromosome 3):

| Position | polymorphism | |
|---|---|---|
| AGPv4 | maintainer | restorer |
| 195629901 | ade | gua |
| 195639694 | thy | cyt |
| 195677799 | gua | ade |
| 195678356 | cyt | gua |
| 195680790 | gua | ade |
| 195732936 | gua | ade |
| 195733916 | cyt | ade |
| 195783701 | cyt | thy |
| 196070086 | cyt | thy |
| 196198736 | ade | gua |
| 196244714 | thy | cyt |
| 196653746 | cyt | thy |
| 196693501 | cyt | ade |
| 196702811 | ade | gua |
| 196704008 | ade | gua |
| 196704096 | thy | cyt |
| 196704169 | thy | cyt |
| 196704290 | thy | cyt |
| 196705470 | thy | ade |
| 196706697 | gua | ade |
| 196706755 | cyt | ade |
| 196707190 | ade | gua |
| 196707415 | thy | ade |
| 196707997 | gua | gua |
| 196773893 | cyt | thy |
| 196774122 | cyt | thy |
| 196774333 | gua | ade |
| 196774502 | gua | ade |
| 196774823 | cyt | thy |
| 196774965 | cyt | gua |
| 196775596 | gua | ade |
| 196776600 | ade | gua |
| 196776877 | cyt | thy |
| 196840068 | cyt | ade |
| 196840815 | gua | cyt |
| 196841649 | gua | cyt |
| 196841990 | cyt | ade |
| 196843002 | cyt | gua |
| 196843335 | cyt | gua |
| 196844084 | cyt | thy |
| 196851451 | thy | cyt |
| 196853534 | gua | cyt |
| 196853762 | cyt | thy |
| 196880373 | cyt | thy |
| 196985856 | cyt | ade |
| 196985883 | cyt | thy |
| 196987284 | cyt | thy |
| 196989025 | gua | thy |
| 196989252 | gua | ade |
| 196989377 | gua | ade |
| 196989408 | ade | gua |
| 197453646 | ade | gua |
| 197453708 | ade | gua |
| 197453708 | thy | cyt |
| 197454448 | gua | ade |
| 197454630 | thy | cyt |
| 197454657 | thy | cyt |
| 197454744 | thy | gua |
| 197454780 | cyt | gua |
| 197454833 | ade | gua |
| 197455007 | cyt | thy |
| 197455034 | thy | cyt |
| 197456922 | thy | cyt |
| 197457134 | ade | gua |
| 197457214 | cyt | thy |
| 197457351 | gua | ade |
| 197457603 | gua | ade |
| 197459188 | thy | cyt |
| 197487965 | gua | ade |
| 197488751 | cyt | thy |
| 197489067 | gua | ade |
| 197524489 | gua | thy |
| 197524855 | cyt | thy |
| 197525193 | gua | ade |
| 197525365 | gua | thy |
| 197525625 | thy | cyt |
| 197525990 | cyt | thy |
| 197526621 | ade | gua |
| 197526690 | gua | ade |
| 197527582 | gua | ade |
| 197527682 | cyt | gua |
| 197528652 | cyt | thy |
| 197556227 | gua | ade |
| 197609686 | gua | thy |
| 197609730 | ade | gua |
| 197611692 | ade | gua |
| 197611832 | gua | ade |
| 197611894 | gua | ade |
| 197613135 | ade | cyt |
| 197613658 | ade | cyt |
| 197613658 | thy | gua |
| 197615089 | gua | ade |
| 197615461 | cyt | thy |
| 197631560 | ade | gua |
| 197631688 | gua | ade |
| 197632590 | gua | ade |
| 197632706 | ade | gua |
| 197633370 | thy | cyt |
| 197633860 | gua | ade |
| 197638778 | thy | cyt |
| 197638949 | thy | cyt |
| 197639380 | gua | ade |
| 197652023 | cyt | thy |
| 197652478 | ade | gua |
| 197653125 | gua | thy |
| 197654542 | cyt | gua |
| 197687270 | cyt | thy |
| 197687524 | cyt | thy |
| 197688212 | gua | cyt |
| 197688445 | thy | cyt |
| 197688492 | thy | cyt |
| 197692991 | ade | gua |
| 197692996 | thy | cyt |
| 197694265 | thy | cyt |
| 197695368 | cyt | thy |
| 197695591 | ade | gua |
| 197695857 | thy | cyt |
| 197696192 | thy | ade |
| 197696732 | gua | thy |
| 197696762 | gua | ade |
| 197697327 | gua | ade |
| 197697514 | cyt | thy |
| 197698249 | gua | ade |
| 197698278 | gua | ade |
| 197708137 | thy | cyt |
| 197708334 | thy | ade |
| 197758073 | thy | gua |
| 197760175 | gua | ade |
| 197761254 | cyt | ade |
| 197761305 | ade | cyt |
| 197776540 | thy | cyt |
| 197777549 | ade | gua |
| 197777618 | thy | ade |
| 197778110 | ade | gua |
| 197781849 | cyt | ade |
| 197781961 | gua | ade |
| 197784696 | ade | gua |
| 197785166 | cyt | thy |
| 197785270 | cyt | gua |
| 197786148 | gua | ade |
| 197786155 | cyt | gua |
| 197787768 | cyt | thy |
| 197806056 | gua | ade |
| 197806483 | cyt | thy |
| 197812595 | gua | ade |
| 197813589 | thy | cyt |
| 197814082 | gua | ade |
| 197840802 | thy | cyt |
| 197840951 | gua | ade |
| 197855989 | cyt | thy |
| 197859323 | gua | cyt |
| 197860711 | ade | cyt |
| 197861373 | gua | ade |
| 197895272 | ade | gua |
| 197902823 | gua | ade |
| 197902855 | cyt | thy |
| 197902923 | cyt | thy |
| 197903119 | cyt | ade |
| 197903264 | thy | cyt |
| 197903302 | gua | cyt |
| 197903372 | cyt | thy |
| 197903475 | cyt | thy |
| 197903587 | gua | thy |
| 197903629 | gua | ade |
| 197903716 | thy | cyt |
| 197903816 | thy | gua |
| 197904016 | thy | thy |
| 197904655 | cy | ade |
| 197906673 | ade | gua |
| 197907566 | gua | ade |
| 197907617 | ade | cyt |
| 197907653 | thy | cyt |
| 197907842 | thy | cyt |
| 197909824 | ade | ade |
| 197948546 | ade | gua |
| 197948580 | cyt | thy |
| 197948690 | cyt | ade |
| 197948831 | gua | gua |
| 197948879 | ade | gua |
| 197973632 | cyt | cyt |
| 197974493 | ade | gua |
| 197994208 | thy | gua |
| 198023432 | thy | cyt |
| 198023573 | cyt | gua |

By means of example, the invention relates to a method for identifying a plant or plant part, in particular a maize plant or plant part, comprising screening for the presence of or detecting or identifying a polymorphism at a position corresponding to maize reference B73 AGPv4 chromosome 3 position 195629901. By means of example, the invention relates to a method for identifying a plant or plant part, in particular a maize plant or plant part, comprising screening for the presence of or detecting or identifying a SNP at a position corresponding to maize reference B73 AGPv4 chromosome 3 position 195629901. By means of example, the invention relates to a method for identifying a plant or plant part, in particular a maize plant or plant part, comprising screening for the presence of or detecting or identifying a SNP at a position corresponding to maize reference B73 AGPv4 chromosome 3 position 195629901, wherein if a G is detected, said maize plant or plant part is a restorer or comprises a restorer (gene(s), locus, haplotype, genome, or phenotype), in particular the restorer of the invention. By means of example, the invention relates to a method for identifying a plant or plant part, in particular a maize plant or plant part, comprising screening for the presence of or detecting or identifying a SNP at a position corresponding to maize reference B73 AGPv4 chromosome 3 position 195629901, wherein if a nucleotide other than G is detected, said maize plant or plant part is a not a restorer or does not comprise a restorer (gene(s), locus, haplotype, genome, or phenotype), in particular the restorer of the invention. By means of example, the invention relates to a method for identifying a plant or plant part, in particular a maize plant or plant part, comprising screening for the presence of or detecting or identifying a SNP at a position corresponding to maize reference B73 AGPv4 chromosome 3 position 195629901, wherein if a nucleotide other than G is detected, said maize plant or plant part is a maintainer or comprise a maintainer (gene(s), locus, haplotype, genome, or phenotype), in particular the maintainer of the invention. By means of example, the invention relates to a method for identifying a plant or plant part, in particular a maize plant or plant part, comprising screening for the presence of or detecting or identifying a SNP at a position corresponding to maize reference B73 AGPv4 chromosome 3 position 195629901, wherein if a nucleotide A is detected, said maize plant or plant part is a not a restorer or does not comprise a restorer (gene(s), locus, haplotype, genome, or phenotype), in particular the restorer of the invention. By means of example, the invention relates to a method for identifying a plant or plant part, in particular a maize plant or plant part, comprising screening for the presence of or detecting or identifying a SNP at a position corresponding to maize reference B73 AGPv4 chromosome 3 position 195629901, wherein if a nucleotide A is detected, said maize plant or plant part is a maintainer or comprise a maintainer (gene(s), locus, haplotype, genome, or phenotype), in particular the maintainer of the invention.

Corresponding embodiments apply to each of the other polymorphisms/SNPs.

It is to be understood that the indicated nucleotide positions are the nucleotide positions of the indicated AGPv04 B73 chromosome 3 positions and that the marker positions in the maize plants according to the invention correspond to the indicated marker positions, but are or comprise not necessarily identical positions in a different genome (e.g. from a different race or line). The skilled person will understand that corresponding nucleotide positions can be determined by suitable alignment, as is known in the art.

The nucleotides (SNPs) at the positions indicated for the restorer allele allow screening for or the identification of the restorer phenotype according to the invention. The nucleotides (SNPs) at the positions indicated for the maintainer allele allow screening for or the identification of the non-restorer phenotype (i.e. the restorer locus at maize chromosome is not present). It will be understood that for identification of the non-restorer allele the indicated SNP nucleotides may be different than those indicated in the Table (as long as these are different than the SNP nucleotides indicated for the restorer allele).

In an aspect, the invention relates to a method for identifying a plant or plant part, in particular a maize plant or plant part, comprising screening for the presence of or detecting or identifying one or more, two or more, or three or more of the following polymorphisms (positions correspond to the indicated positions of the respective SEQ ID NOs):

| | | | Position | | |
|---|---|---|---|---|---|
| | Posi- | | B73 | | |
| SEQ | tion | SEQ | (AGPv4) = | polymorphism | |
| ID | re- | ID | main- | re- | main- |
| NO: | storer | NO: | tainer | storer | tainer |
| 1 | 35 | 2 | 35 | g | t |
| 1 | 404 | 2 | 404 | t | c |
| 1 | 444- | 2 | 443- | gggac | cg |
| | 452 | | 444 | tttc | |
| 1 | 463 | 2 | 454 | c | t |
| 1 | 537 | 2 | 528 | g | c |
| 1 | 735 | 2 | 726 | g | a |
| 1 | 748- | 2 | 738- | tacttt | at |
| | 759 | | 739 | gtaaca | |
| 1 | 761 | 2 | 740 | t | a |
| 1 | 797 | 2 | 776 | a | g |
| 1 | 1048 | 2 | 1027 | g | t |
| 1 | 1056 | 2 | 1035 | a | c |
| 1 | 1065- | 2 | 1045 | tc | a |
| | 1066 | | | | |
| 1 | 1072- | 2 | 1053- | cc | ctt |
| | 1073 | | 1059 | | ctcc |
| 1 | 1071 | 2 | 1058 | g | c |
| 1 | 1188 | 2 | 1175 | c | t |
| 1 | 1218 | 2 | 1208 | t | c |
| 1 | 1765 | 2 | 1757 | a | g |
| 1 | 1769 | 2 | 1761 | g | a |
| 1 | 1844 | 2 | 1836 | t | g |
| 1 | 1856 | 2 | 1848 | t | a |
| 1 | 2076 | 2 | 2068 | c | t |
| 1 | 2089 | 2 | 2081 | g | a |
| 1 | 2146 | 2 | 2138 | t | c |
| 1 | 2168- | 2 | 2161 | tt | t |
| | 2169 | | | | |
| 1 | 2214 | 2 | 2207 | g | t |
| 1 | 2370 | 2 | 2362- | c | ct |
| | | | 2363 | | |
| 1 | 2582 | 2 | 2574 | c | g |
| 1 | 2632- | 2 | 2624- | tac | cca |
| | 2637 | | 2629 | tgt | ctg |
| 1 | 2641 | 2 | 2633 | a | t |
| 1 | 2643- | 2 | 2635- | cg | ac |
| | 2644 | | 2636 | | |
| 1 | 2696 | 2 | 2688 | t | c |
| 1 | 2738 | 2 | 2730 | c | a |
| 1 | 2843 | 2 | 2834- | g | gt |
| | | | 2835 | | |
| 1 | 2849 | 2 | 2840 | t | c |
| 1 | 2954- | 2 | 2946- | tt | tt |
| | 2955 | | 2947 | | |
| 1 | 3004 | 2 | 2997 | g | a |
| 1 | 3047 | 2 | 3040 | a | t |
| 1 | 3068 | 2 | 3061 | c | a |
| 1 | 3218 | 2 | 3211 | a | g |
| 5 | 486 | 6 | 486 | a | t |
| 5 | 611 | 6 | 611 | g | a |
| 5 | 638 | 6 | 638 | a | g |
| 5 | 689 | 6 | 689 | g | a |
| 5 | 812 | 6 | 812 | c | t |
| 5 | 865 | 6 | 865 | c | g |
| 5 | 901 | 6 | 901 | c | a |
| 5 | 988 | 6 | 988 | g | a |
| 5 | 1015 | 6 | 1015 | c | t |
| 5 | 1085 | 6 | 1085 | c | t |
| 5 | 1197 | 6 | 1197 | t | c |
| 5 | 1345 | 6 | 1345 | c | t |
| 5 | 1461 | 6 | 1461 | c | t |
| 5 | 1937 | 6 | 1937 | c | t |
| 5 | 1999 | 6 | 1999 | c | t |
| 5 | 2113- | 6 | 2112- | gca | gt |
| | 2115 | | 2113 | | |
| 5 | 2286 | 6 | 2283 | c | t |
| 5 | 2293- | 6 | 2289- | ct | gt |
| | 2297 | | 2290 | acg | |
| 5 | 2399 | 6 | 2391 | c | g |
| 5 | 2448- | 6 | 2439- | ctc | cc |
| | 2450 | | 2440 | | |
| 5 | 2822- | 6 | 2810- | tt | tt |
| | 2823 | | 2811 | | |
| 5 | 2856 | 6 | 2843 | g | a |
| 5 | 2926 | 6 | 2913 | a | g |
| 5 | 2998 | 6 | 2985 | t | c |
| 5 | 3029 | 6 | 3016 | t | c |
| 5 | 3085 | 6 | 3072 | t | c |
| 5 | 3102 | 6 | 3089 | t | c |
| 5 | 3112 | 6 | 3099 | c | a |
| 5 | 3120 | 6 | 3107 | t | g |
| 5 | 3168 | 6 | 3155 | t | a |
| 9 | 392- | 10 | 393- | tg | gtg |
| | 393 | | 397 | | gt |
| 9 | 550 | 10 | 555 | g | a |
| 9 | 591 | 10 | 596 | t | g |
| 9 | 886- | 10 | 891- | ct | tc |
| | 887 | | 892 | | |
| 9 | 934 | 10 | 939 | a | g |
| 9 | 957 | 10 | 962 | t | c |
| 9 | 1097 | 10 | 1102 | c | t |
| 9 | 1130 | 10 | 1135 | c | t |
| 9 | 1295 | 10 | 1300 | a | g |
| 9 | 1462 | 10 | 1467 | t | c |
| 9 | 1467 | 10 | 1472 | t | g |
| 9 | 1541 | 10 | 1546 | g | a |
| 9 | 1584 | 10 | 1603 | g | t |
| 9 | 1614 | 10 | 1633 | c | t |
| 9 | 1713 | 10 | 1732 | c | t |
| 9 | 1774 | 10 | 1793 | a | g |
| 9 | 1795- | 10 | 1815- | tt | tttt |
| | 1796 | | 1823 | | tgttt |
| 9 | 1815 | 10 | 1843 | a | c |
| 9 | 1894- | 10 | 1923 | tt | t |
| | 1895 | | | | |
| 9 | 1910 | 10 | 1939 | a | t |
| 9 | 1954- | 10 | 1984- | gt | tttg |
| | 1955 | | 1991 | | acac |
| 9 | 2000 | 10 | 2037 | t | c |
| 9 | 2060 | 10 | 2097 | t | c |
| 9 | 2181 | 10 | 2218 | c | t |
| 9 | 2354 | 10 | 2391 | a | g |
| 9 | 2372 | 10 | 2409 | a | t |
| 9 | 2394- | 10 | 2431- | ctg | aaca |
| | 2399 | | 2434 | ttt | |
| 9 | 2428 | 10 | 2463 | a | g |
| 9 | 2439- | 10 | 2475- | ct | tt |
| | 2440 | | 2476 | | |
| 13 | 651- | 14 | 652- | cc | cgc |
| | 652 | | 654 | | |
| 13 | 807 | 14 | 810 | t | c |

By means of example, the invention relates to a method for identifying a plant or plant part, in particular a maize plant or plant part, comprising screening for the presence of or detecting or identifying a polymorphism at a position corresponding to position 35 of SEQ ID NO: 1, or a sequence which is at least 80%, preferably at least 85%, more preferably at least 90%, most preferably at least 95% identical to a sequence as set forth in SEQ ID NO: 1 (and comprising the polymorphism). By means of example, the invention relates to a method for identifying a plant or plant part, in particular a maize plant or plant part, comprising screening for the presence of or detecting or identifying a polymorphism at a position corresponding to position 35 of SEQ ID NO: 2, or a sequence which is at least 80%, preferably at least 85%, more preferably at least 90%, most preferably at least 95% identical to a sequence as set forth in SEQ ID NO: 2 (and comprising the polymorphism).

By means of example, the invention relates to a method for identifying a plant or plant part, in particular a maize plant or plant part, comprising screening for the presence of or detecting or identifying a polymorphism at a position corresponding to position 35 of SEQ ID NO: 1, or a sequence which is at least 80%, preferably at least 85%, more preferably at least 90%, most preferably at least 95% identical to a sequence as set forth in SEQ ID NO: 1 (and comprising the polymorphism), wherein if a G is detected said maize plant or plant part is a maintainer or is not a restorer or comprises a maintainer (gene(s), locus, haplotype, genome, or phenotype) or does not comprise a restorer (gene(s), locus, haplotype, genome, or phenotype), in particular the maintainer/restorer of the invention. By means of example, the invention relates to a method for identifying a plant or plant part, in particular a maize plant or plant part, comprising screening for the presence of or detecting or identifying a polymorphism at a position corresponding to position 35 of SEQ ID NO: 2, or a sequence which is at least 80%, preferably at least 85%, more preferably at least 90%, most preferably at least 95% identical to a sequence as set forth in SEQ ID NO: 2 (and comprising the polymorphism), wherein if a T is detected said maize plant or plant part is restorer or comprises a restorer (gene(s), locus, haplotype, genome, or phenotype), in particular the restorer of the invention.

Corresponding embodiments apply to each of the other polymorphisms, SNPs, insertions, deletions, and substitutions.

Screening for the respective polymorphisms can be achieved by means knows in the art, such as for instance KASP, as described herein elsewhere. KASP primers may for instance be developed to discriminate between the restorer and non-restorer/maintainer polymorphisms.

The methods for identifying a (maize) plant or plant part as described herein may comprise screening of a sample obtained from a (maize) plant or plant part, in particular a sample comprising genomic DNA of the (maize) plant or plant part. Accordingly, the method may comprise the step of obtaining a sample (comprising genomic DNA) from a (maize) plant or plant part, or providing a sample (comprising genomic DNA) obtained from a (maize) plant or plant part. Methods for screening or identifying markers are well known in the art, as also described herein elsewhere.

It will be understood that the methods for identifying a (maize) plant or plant part as described herein allow for discriminating between plants or plant parts having a cytoplasmic male sterility restorer or a cytoplasmic male sterility maintainer genotype, haplotype, and/or phenotype based on the identity of the polymorphisms or polymorphic alleles described herein. Accordingly, the molecular marker(s) (allele(s)) of the present invention can be advantageously used to identify maize plants as being a restorer or having a restorer gene, locus (allele), haplotype, genotype or phenotype or as not being a restorer or not having a restorer gene, locus (allele), haplotype, genotype or phenotype, in particular the restorer of the invention. As also described herein elsewhere, such plants or plant parts may nevertheless comprise other restorer genes or loci.

In an aspect, the methods for identifying a (maize) plant or plant part as described herein are methods for discriminating between a (maize) plant or plant part having the cytoplasmic male sterility restorer of the invention as described herein elsewhere and a (maize) plant or plant part lacking the cytoplasmic male sterility restorer of the invention as described herein elsewhere. In an aspect, the methods for identifying a (maize) plant or plant part as described herein are methods for identifying a (maize) plant or plant part having the cytoplasmic male sterility restorer of the invention as described herein elsewhere. In an aspect, the methods for identifying a (maize) plant or plant part as described herein are methods for identifying a (maize) plant or plant part lacking the cytoplasmic male sterility restorer of the invention as described herein elsewhere.

In certain embodiments, a (maize) plant or plant part is identified as having the cytoplasmic male sterility restorer of the invention as described herein elsewhere if the restorer locus, haplotype, marker(s) (allele(s)), SNPs, etc. are detected (in the genome of the plant or plant part), e.g. as provided in Tables 4 or 5, SEQ ID NOs: 1, 5, 9, o 13, etc. In certain embodiments, a (maize) plant or plant part is identified as lacking the cytoplasmic male sterility restorer of the invention as described herein elsewhere if the restorer locus, haplotype, marker(s) (allele(s)), SNPs, etc. are not detected (in the genome of the plant or plant part), e.g. as provided in Tables 4 or 5, SEQ ID NOs: 1, 5, 9, o 13, etc. In certain embodiments, a (maize) plant or plant part is identified as lacking the cytoplasmic male sterility restorer of the invention as described herein elsewhere if the maintainer locus, haplotype, marker(s) (allele(s)), SNPs, etc. are detected (in the genome of the plant or plant part), e.g. as provided in Tables 4 or 5, SEQ ID NOs: 2, 6, 10, o 14, etc.

Underlying the present invention is the identification of a CMS (cytoplasmic male sterility) restorer locus, in particular located on maize chromosome 3. Accordingly, the methods for identifying plants or plant parts as described herein can be methods for identifying plants or plant parts comprising said CMS restorer locus or alternatively methods for identifying plants or plant parts not comprising said CMS restorer locus. Such identification can be based on the polymorphisms described herein, in particular the polymorphic alleles associated with/linked with the restorer locus or alternatively the polymorphic alleles associated with/linked with the maintainer locus.

Accordingly, the methods for identifying plants or plant parts as described herein can be methods for identifying plants or plant parts comprising the CMS maintainer locus or alternatively methods for identifying plants or plant parts not comprising the CMS maintainer locus.

In certain embodiments, the restorer locus of the invention is comprised on maize chromosome 3 in a genomic interval corresponding to (nucleic acid) position 195629901 to 198023573 of the B73 reference maize genome AGPv4, or a fragment thereof. In certain embodiments, the restorer locus comprises on maize chromosome 3 a genomic interval corresponding to (nucleic acid) position 195629901 to 198023573 of the B73 reference maize genome AGPv4, or a fragment thereof. In certain embodiments, the restorer locus is flanked on maize chromosome 3 by (nucleic acid) positions corresponding to positions 195629901 and 198023573 of the B73 reference maize genome AGPv4, or a fragment thereof.

In certain embodiments, the restorer locus of the invention is comprised on maize chromosome 3 in a genomic interval corresponding to (nucleic acid) position 197453646 to 197698278 of the B73 reference maize genome AGPv4, or a fragment thereof. In certain embodiments, the restorer locus comprises on maize chromosome 3 a genomic interval corresponding to (nucleic acid) position 197453646 to 197698278 of the B73 reference maize genome AGPv4, or a fragment thereof. In certain embodiments, the restorer locus is flanked on maize chromosome 3 by (nucleic acid) positions corresponding to positions 197453646 and 197698278 of the B73 reference maize genome AGPv4, or a fragment thereof.

In certain embodiments, the restorer locus of the invention is comprised on maize chromosome 3 in a genomic interval corresponding to (nucleic acid) position 195629901 to 197698278 of the B73 reference maize genome AGPv4, or a fragment thereof. In certain embodiments, the restorer locus comprises on maize chromosome 3 a genomic interval corresponding to (nucleic acid) position 195629901 to 197698278 of the B73 reference maize genome AGPv4, or a fragment thereof. In certain embodiments, the restorer locus is flanked on maize chromosome 3 by (nucleic acid) positions corresponding to positions 195629901 and 197698278 of the B73 reference maize genome AGPv4, or a fragment thereof.

In certain embodiments, the restorer locus of the invention is comprised on maize chromosome 3 in a genomic interval corresponding to (nucleic acid) position 197453646 to 198023573 of the B73 reference maize genome AGPv4, or a fragment thereof. In certain embodiments, the restorer locus comprises on maize chromosome 3 a genomic interval corresponding to (nucleic acid) position 197453646 to 198023573 of the B73 reference maize genome AGPv4, or a fragment thereof. In certain embodiments, the restorer locus is flanked on maize chromosome 3 by (nucleic acid) positions corresponding to positions 197453646 and 198023573 of the B73 reference maize genome AGPv4, or a fragment thereof.

In certain embodiments, the molecular marker(s) (allele(s)) are selected from Table 4 or 5. As indicated in Tables 4 and 5, all markers are polymorphic and are capable of discriminating between the restorer and non-restorer (or maintainer). Accordingly, identification of a restorer entails identification of one or more restorer-associated/linked polymorphisms as indicated in Tables 4 and 5, whereas identification of a non-restorer/maintainer entails identification of one or more non-restorer/maintainer-associated/linked polymorphisms as indicated in Tables 4 and 5.

As referred to herein, a polynucleic acid or locus of the invention as described herein, is said to be flanked by certain molecular markers or molecular marker alleles if the polynucleic acid/locus is comprised within a polynucleic acid wherein respectively a first marker (allele) is located upstream (i.e. 5′) of said polynucleic acid and a second marker (allele) is located downstream (i.e. 3′) of said polynucleic acid. Such first and second marker (allele) may border the polynucleic acid. The nucleic acid may equally comprise such first and second marker (allele), such as respectively at or near the 5′ and 3′ end, for instance respectively within 50 kb of the 5′ and 3′ end, preferably within 10 kb of the 5′ and 3′ end, such as within 5 kb of the 5′ and 3′ end, within 1 kb of the 5′ and 3′ end, or less.

In certain embodiments, the methods of the invention for identifying a maize plant or plant part comprise screening for the presence of or detecting or identifying one or more molecular marker (allele) selected from SEQ ID Nos: 17 to 200, wherein n is the corresponding nucleotide for the restorer polymorphism (SNP) (as indicated in Table 4).

In certain embodiments, the methods of the invention for identifying a maize plant or plant part comprise screening for the presence of or detecting or identifying one or more molecular marker (allele) selected from SEQ ID Nos: 17 to 200, wherein n is the corresponding nucleotide for the non-restorer/maintainer polymorphism (SNP) (as indicated in Table 4).

In certain embodiments, the methods of the invention for identifying a maize plant or plant part comprise screening for the absence of one or more molecular marker (allele) selected from SEQ ID Nos: 17 to 200, wherein n is the corresponding nucleotide for the restorer polymorphism (SNP) (as indicated in Table 4).

In certain embodiments, the methods of the invention for identifying a maize plant or plant part comprise screening for the absence of one or more molecular marker (allele) selected from SEQ ID Nos: 17 to 200, wherein n is the corresponding nucleotide for the non-restorer/maintainer polymorphism (SNP) (as indicated in Table 4).

In certain embodiments, the methods of the invention for identifying a maize plant or plant part comprise screening for the presence of or detecting or identifying one or more molecular marker (allele) selected from SEQ ID Nos: 68 to 140, wherein n is the corresponding nucleotide for the restorer polymorphism (SNP) (as indicated in Table 4).

In certain embodiments, the methods of the invention for identifying a maize plant or plant part comprise screening for the presence of or detecting or identifying one or more molecular marker (allele) selected from SEQ ID Nos: 68 to 140, wherein n is the corresponding nucleotide for the non-restorer/maintainer polymorphism (SNP) (as indicated in Table 4).

In certain embodiments, the methods of the invention for identifying a maize plant or plant part comprise screening for the absence of one or more molecular marker (allele) selected from SEQ ID Nos: 68 to 140, wherein n is the corresponding nucleotide for the restorer polymorphism (SNP) (as indicated in Table 4).

In certain embodiments, the methods of the invention for identifying a maize plant or plant part comprise screening for the absence of one or more molecular marker (allele) selected from SEQ ID Nos: 68 to 140, wherein n is the corresponding nucleotide for the non-restorer/maintainer polymorphism (SNP) (as indicated in Table 4).

In certain embodiments, the methods of the invention for identifying a maize plant or plant part comprise screening for the presence of or detecting or identifying one or more molecular marker (allele) selected from SEQ ID Nos: 70, 72, 76, 79, 86, 88, 92, 93, 99, 104, 105, 107, 108, 109, 115, and 134, wherein n is the corresponding nucleotide for the restorer polymorphism (SNP) (as indicated in Table 4).

In certain embodiments, the methods of the invention for identifying a maize plant or plant part comprise screening for the presence of or detecting or identifying one or more molecular marker (allele) selected from SEQ ID Nos: 70, 72, 76, 79, 86, 88, 92, 93, 99, 104, 105, 107, 108, 109, 115, and 134, wherein n is the corresponding nucleotide for the non-restorer/maintainer polymorphism (SNP) (as indicated in Table 4).

In certain embodiments, the methods of the invention for identifying a maize plant or plant part comprise screening for the absence of one or more molecular marker (allele) selected from SEQ ID Nos: 70, 72, 76, 79, 86, 88, 92, 93, 99, 104, 105, 107, 108, 109, 115, and 134, wherein n is the corresponding nucleotide for the restorer polymorphism (SNP) (as indicated in Table 4).

In certain embodiments, the methods of the invention for identifying a maize plant or plant part comprise screening for the absence of one or more molecular marker (allele) selected from SEQ ID Nos: 70, 72, 76, 79, 86, 88, 92, 93, 99, 104, 105, 107, 108, 109, 115, and 134, wherein n is the corresponding nucleotide for the non-restorer/maintainer polymorphism (SNP) (as indicated in Table 4).

In certain embodiments, the methods of the invention for identifying a maize plant or plant part comprise screening for the presence of or detecting or identifying any one or more of SEQ ID NOs: 1, 5, 9, and 13, or a (unique) fragment thereof, or a sequence which is at least 80%, preferably at least 85%, more preferably at least 90%, most preferably at least 95% identical to a sequence as set forth in any one or more of SEQ ID NOs: 1, 5, 9, and 13, or a (unique) fragment thereof, or one or more of SEQ ID NOs: 2, 6, 10, and 14, or a (unique) fragment thereof, or a sequence which is at least 80%, preferably at least 85%, more preferably at least 90%, most preferably at least 95% identical to a sequence as set forth in any one or more of SEQ ID NOs: 2, 6, 10, and 14, or a (unique) fragment thereof. In certain embodiments, the methods of the invention for identifying a maize plant or plant part comprise screening for the presence of or detecting or identifying any one or more of SEQ ID NOs: 1, 5, 9, and 13, or a (unique) fragment thereof, or a sequence which is at least 80%, preferably at least 85%, more preferably at least 90%, most preferably at least 95% identical to a sequence as set forth in any one or more of SEQ ID NOs: 1, 5, 9, and 13, or a (unique) fragment thereof, wherein the presence indicates that the plant or plant part is a restorer or comprises a restorer (gene(s), locus (allele), haplotype, genome, or phenotype). In certain embodiments, the methods of the invention for identifying a maize plant or plant part comprise screening for the presence of or detecting or identifying any one or more of SEQ ID NOs: 1, 5, 9, and 13, or a (unique) fragment thereof, or a sequence which is at least 80%, preferably at least 85%, more preferably at least 90%, most preferably at least 95% identical to a sequence as set forth in any one or more of SEQ ID NOs: 1, 5, 9, and 13, or a (unique) fragment thereof, wherein the absence indicates that the plant or plant part is a not a restorer or does not comprise a restorer (gene(s), locus (allele), haplotype, genome, or phenotype). In certain embodiments, the methods of the invention for identifying a maize plant or plant part comprise screening for the presence of or detecting or identifying any one or more of SEQ ID NOs: 1, 5, 9, and 13, or a (unique) fragment thereof, or a sequence which is at least 80%, preferably at least 85%, more preferably at least 90%, most preferably at least 95% identical to a sequence as set forth in any one or more of SEQ ID NOs: 1, 5, 9, and 13, or a (unique) fragment thereof, wherein the absence indicates that the plant or plant part is a maintainer or comprises a maintainer (gene(s), locus (allele), haplotype, genome, or phenotype).

In certain embodiments, the methods of the invention for identifying a maize plant or plant part comprise screening for the presence of or detecting or identifying any one or more of SEQ ID NOs: 2, 6, 10, and 14, or a (unique) fragment thereof, or a sequence which is at least 80%, preferably at least 85%, more preferably at least 90%, most preferably at least 95% identical to a sequence as set forth in any one or more of SEQ ID NOs: 2, 6, 10, and 14, or a (unique) fragment thereof, wherein the presence indicates that the plant or plant part is not a restorer or does not comprise a restorer (gene(s), locus (allele), haplotype, genome, or phenotype). In certain embodiments, the methods of the invention for identifying a maize plant or plant part comprise screening for the presence of or detecting or identifying any one or more of SEQ ID NOs: 2, 6, 10, and 14, or a (unique) fragment thereof, or a sequence which is at least 80%, preferably at least 85%, more preferably at least 90%, most preferably at least 95% identical to a sequence as set forth in any one or more of SEQ ID NOs: 2, 6, 10, and 14, or a (unique) fragment thereof, wherein the presence indicates that the plant or plant part is a maintainer or comprises a maintainer (gene(s), locus (allele), haplotype, genome, or phenotype). In certain embodiments, the methods of the invention for identifying a maize plant or plant part comprise screening for the presence of or detecting or identifying any one or more of SEQ ID NOs: 2, 6, 10, and 14, or a (unique) fragment thereof, or a sequence which is at least 80%, preferably at least 85%, more preferably at least 90%, most preferably at least 95% identical to a sequence as set forth in any one or more of SEQ ID NOs: 2, 6, 10, and 14, or a (unique) fragment thereof, wherein the absence indicates that the plant or plant part is a restorer or comprises a restorer (gene(s), locus (allele), haplotype, genome, or phenotype).

In an aspect, the invention relates to an (isolated) polynucleic acid comprising or consisting of any one or more of the sequences, molecular markers or molecular marker alleles as described herein elsewhere, or a fragment thereof, and/or the complement thereof or the reverse complement thereof.

In certain embodiments, the polynucleotide or polynucleic acid according to the invention as described herein is an isolated polynucleotide or polynucleic acid.

In an aspect, the invention relates to an (isolated) polynucleic acid comprising or consisting of a (unique) fragment of any of the sequences, molecular markers or molecular marker alleles as described herein elsewhere, or the complement thereof or the reverse complement thereof. Preferably said polynucleic acid is at least 15 nucleotides, more preferably at least 20 nucleotides, such as at least 25, 30, 35, 40, 45, 50, 75, 100, 125, 150, 175, 200 or more nucleotides. In certain embodiments, the polynucleic acid is at most 500 nucleotides, preferably at most 250 nucleotides, such as at most 200, 150, 100, or 50 nucleotides. In certain embodiments, the polynucleic acid has from 15 to 500 nucleotides, such as from 20 to 250 nucleotides or from 20 to 100 nucleotides, such as from 20 to 50 nucleotides.

In an aspect, the invention relates to an (isolated) polynucleic acid (specifically) hybridizing with any of the sequences, molecular markers or molecular marker alleles as described herein elsewhere, or the complement thereof or the reverse complement thereof. Preferably said polynucleic acid is at least 15 nucleotides, more preferably at least 20 nucleotides, such as at least 25, 30, 35, 40, 45, 50, 75, 100, 125, 150, 175, 200 or more nucleotides. In certain embodiments, the polynucleic acid is at most 500 nucleotides, preferably at most 250 nucleotides, such as at most 200, 150, 100, or 50 nucleotides. In certain embodiments, the polynucleic acid has from 15 to 500 nucleotides, such as from 20 to 250 nucleotides or from 20 to 100 nucleotides, such as from 20 to 50 nucleotides.

In an aspect, the invention relates to a polynucleic acid comprising a molecular marker (allele) of Table 4 or Table 5, or a (unique) fragment thereof, and/or the complement or reverse complement thereof. In an aspect, the invention relates to a polynucleic acid comprising a restorer molecular marker (allele) of Table 4 or Table 5, or a (unique) fragment thereof, and/or the complement or reverse complement thereof. In an aspect, the invention relates to a polynucleic acid comprising a non-restorer/maintainer molecular marker (allele) of Table 4 or Table 5, or a (unique) fragment thereof, and/or the complement or reverse complement thereof.

According to certain embodiments, when reference is made to a fragment of a polynucleic acid or protein, such fragment comprises respectively at least 15 nucleotides or amino acids, preferably at least 20 nucleotides or amino acids.

It will be understood that the polynucleic acids according to the invention comprises or specifically hybridizes with one or more of the molecular marker (allele) and additional 5′ and/or 3′ contiguous nucleotides (naturally) flanking the respective marker (allele) (or the complement or reverse complement thereof). In this context, the amount of flanking may in certain embodiments be at least 14 or 15 nucleotides (which may or may not be entirely 5′ or entirely 3′ flanking nucleotides, such as for instance 5 3′ flanking nucleotides plus 10 5′ flanking nucleotides. In certain embodiments, the molecular marker (allele) of the present invention (or the complement thereof) is the most 5′ nucleotide of the polynucleic acid. In certain embodiments, the molecular marker (allele) of the present invention (or the complement thereof) is the second most 5′ nucleotide of the polynucleic acid. In certain embodiments, the molecular marker (allele) of the present invention (or the complement thereof) is the third most 5′ nucleotide of the polynucleic acid. In certain embodiments, the molecular marker (allele) of the present invention (or the complement thereof) is the most 3′ nucleotide of the polynucleic acid. In certain embodiments, the molecular marker (allele) of the present invention (or the complement thereof) is the second most 3′ nucleotide of the polynucleic acid. In certain embodiments, the molecular marker (allele) of the present invention (or the complement thereof) is the third most 3′ nucleotide of the polynucleic acid. Such terminally located markers (such as SNPs) advantageously allow for the development of allele specific primers, such as for use in KASP.

In an aspect, the invention relates to a polynucleic acid comprising or comprised in any of SEQ ID NOs: 1 to 208, or a (unique) fragment thereof, and/or the complement thereof or the reverse complement thereof. In an aspect, the invention relates to a polynucleic acid specifically hybridizing with a polynucleic acid comprising or comprised in any of SEQ ID NOs: 1 to 208, or a (unique) fragment thereof, and/or the complement thereof or the reverse complement thereof. It will be understood that such polynucleic acids comprise at least one or more of the polymorphic nucleotides, insertions, deletions, or substitutions of the invention as referred to herein elsewhere and contiguous 5′ and/or 3″ flanking sequences, as described herein elsewhere.

In certain embodiments, the polynucleic acid comprises or is comprised in any of SEQ ID NOs: 17 to 200, wherein n is the corresponding nucleotide for the restorer polymorphism (SNP) (as indicated in Table 4).

In certain embodiments, the polynucleic acid comprises or is comprised in any of SEQ ID Nos: 17 to 200, wherein n is the corresponding nucleotide for the non-restorer/maintainer polymorphism (SNP) (as indicated in Table 4).

In certain embodiments, the polynucleic acid comprises or is comprised in any of SEQ ID Nos: 68 to 140, wherein n is the corresponding nucleotide for the restorer polymorphism (SNP) (as indicated in Table 4).

In certain embodiments, the polynucleic acid comprises or is comprised in any of SEQ ID Nos: 68 to 140, wherein n is the corresponding nucleotide for the non-restorer/maintainer polymorphism (SNP) (as indicated in Table 4).

In certain embodiments, the polynucleic acid comprises or is comprised in any of SEQ ID Nos: 70, 72, 76, 79, 86, 88, 92, 93, 99, 104, 105, 107, 108, 109, 115, and 134, wherein n is the corresponding nucleotide for the restorer polymorphism (SNP) (as indicated in Table 4).

In certain embodiments, the polynucleic acid comprises or is comprised in any of SEQ ID Nos: 70, 72, 76, 79, 86, 88, 92, 93, 99, 104, 105, 107, 108, 109, 115, and 134, wherein n is the corresponding nucleotide for the non-restorer/maintainer polymorphism (SNP) (as indicated in Table 4).

In certain embodiments, the polynucleic acid comprises or is comprised in any of SEQ ID NOs: 1-3, 5-7, 9-11, 13-15, 201, 203, 205, or 207, or the complement thereof, or the reverse complement thereof, or a (unique) fragment thereof, or comprises or is comprised in a sequence which is at least 80%, preferably at least 85%, more preferably at least 90%, most preferably at least 95% identical to a sequence as set forth in any one or more of SEQ ID NOs: 1-3, 5-7, 9-11, 13-15, 201, 203, 205, or 207, or the complement thereof, or the reverse complement thereof, or a (unique) fragment thereof. In certain embodiments, the polynucleic acid comprises or is comprised in a polynucleic acid encoding a protein of SEQ ID NOs: 4, 8, 12, 16, 202, 204, 206, 208, or the complement thereof, or the reverse complement thereof, or a (unique) fragment thereof, or comprises or is comprised in a polynucleic acid encoding a protein having a sequence which is at least 80%, preferably at least 85%, more preferably at least 90%, most preferably at least 95% identical to a sequence as set forth in any one or more of SEQ ID NOs: 4, 8, 12, 16, 202, 204, 206, 208, or the complement thereof, or the reverse complement thereof, or a (unique) fragment thereof. In certain embodiments, the polynucleic acid comprises or is comprised in any of SEQ ID NOs: 1, 5, 9, 13, 201, 203, 205, or 207, preferably SEQ ID NO: 1 or 201, or the complement thereof, or the reverse complement thereof, or a (unique) fragment thereof, or comprises or is comprised in a sequence which is at least 80%, preferably at least 85%, more preferably at least 90%, most preferably at least 95% identical to a sequence as set forth in any one or more of SEQ ID NOs: 11, 5, 9, 13, 201, 203, 205, or 207, preferably SEQ ID NO: 1 or 201, or the complement thereof, or the reverse complement thereof, or a (unique) fragment thereof. In certain embodiments, the polynucleic acid comprises or is comprised in a polynucleic acid encoding a protein of SEQ ID NOs: 202, 204, 206, 208, preferably SEQ ID NO: 202, or the complement thereof, or the reverse complement thereof, or a (unique) fragment thereof, or comprises or is comprised in a polynucleic acid encoding a protein having a sequence which is at least 80%, preferably at least 85%, more preferably at least 90%, most preferably at least 95% identical to a sequence as set forth in any one or more of SEQ ID NOs: 202, 204, 206, 208, preferably SEQ ID NO: 202, or the complement thereof, or the reverse complement thereof, or a (unique) fragment thereof. In certain embodiments, the polynucleic acid comprises or is comprised in any of SEQ ID NOs: 2-3, 6-7, 10-11, 14-15, preferably SEQ ID NO: 2, or the complement thereof, or the reverse complement thereof, or a (unique) fragment thereof, or comprises or is comprised in a sequence which is at least 80%, preferably at least 85%, more preferably at least 90%, most preferably at least 95% identical to a sequence as set forth in any one or more of SEQ ID NOs: 2-3, 6-7, 10-11, 14-15, preferably SEQ ID NO: 2, or the complement thereof, or the reverse complement thereof, or a (unique) fragment thereof. In certain embodiments, the polynucleic acid comprises or is comprised in a polynucleic acid encoding a protein of SEQ ID NOs: 4, 8, 12, 16, preferably SEQ ID NO: 4, or the complement thereof, or the reverse complement thereof, or a (unique) fragment thereof, or comprises or is comprised in a polynucleic acid encoding a protein having a sequence which is at least 80%, preferably at least 85%, more preferably at least 90%, most preferably at least 95% identical to a sequence as set forth in any one or more of SEQ ID NOs: 4, 8, 12, 16, preferably SEQ ID NO: 4, or the complement thereof, or the reverse complement thereof, or a (unique) fragment thereof. Such polynucleic acids are suitable for identifying plants or plant parts as well as for generating plants as described herein elsewhere.

In certain embodiments, the polynucleic acid comprises or consists of at least 15 nucleotides, such as 16, 17, 18, 19, 20, 21, 22, 23, 24, or 25 nucleotides, such as at least 30, 35, 40, 45, or 50 nucleotides, such as at least 100, 200, 300, or 500 nucleotides.

In certain embodiments, the polynucleic acid comprises or consists of a polynucleic acid as defined in numbered statements 27 to 30, referred to herein elsewhere.

In certain embodiments, the polynucleic acid comprises or consists of at most 1500 nucleotides, such as 1200, 1000, 800, 600, 400, 200 nucleotides, such as at most 100, 80, 60, 50, 40, or 30 nucleotides.

In certain embodiments, the polynucleic acid comprises or consists of at least 15 nucleotides, such as 16, 17, 18, 19, 20, 21, 22, 23, 24, or 25 nucleotides, such as at least 30, 35, 40, 45, or 50 nucleotides, such as at least 100, 200, 300, or 500 nucleotides, and the polynucleic acid comprises at most 1500 nucleotides, such as 1200, 1000, 800, 600, 400, 200 nucleotides, such as at most 100, 80, 60, 50, 40, or 30 nucleotides.

In certain embodiments, the (isolated) polynucleotide has a length ranging from 15 to 500 nucleotides, preferably 15 to 100 nucleotides, preferably 15 to 50 nucleotides, more preferably 15 to 35 nucleotides.

In certain embodiments, the (isolated) polynucleotide is a primer or a probe.

In certain embodiments, the (isolated) polynucleotide is an allele-specific primer or probe.

In certain embodiments, the (isolated) is a KASP (Kompetitive allele specific PCR) primer. Primers, including KASP primers, are well-known in the art and can be designed by the skilled person according to known criteria. By means of further guidance, and without limitation, KASP is performed with two (or more) allele-specific primers (which may be the forward primers) and generally one common primer (which may be the reverse primer). The allele-specific primers are typically elongated with tail sequences (in which a different tail sequence is provided for each allele-specific primer). The tail sequences allow incorporation of a fluorescently labelled complementary sequence, to thereby fluorescently distinguish the different alleles.

In certain embodiments, the length of the tail sequence is comprised in the total primer length. In certain embodiments, the length of the tail sequence is not comprised in the total primer length.

In certain embodiments, the polynucleic acid is a (PCR) primer or (hybridization) probe. In certain embodiments, the polynucleic acid is an allele-specific primer or probe. In certain embodiments, the polynucleic acid is a KASP primer.

In an aspect, the invention relates to a (isolated) polynucleic acid comprising a (molecular) marker (allele) of the invention, or the complement or the reverse complement of a (molecular) marker (allele) of the invention. In certain embodiments, the invention relates to a polynucleic acid comprising at least 10 contiguous nucleotides, preferably at least 15 contiguous nucleotides or at least 20 contiguous nucleotides of a (molecular) marker (allele) of the invention, or the complement or the reverse complement of a (molecular) marker (allele) of the invention. In certain embodiments, the polynucleic acid is capable of discriminating between a (molecular) marker (allele) of the invention and a non-molecular marker allele, such as to specifically hybridise with a (molecular) marker allele of the invention. In certain embodiments, the polynucleic acid or the complement or reverse complement thereof does not (substantially) hybridise with or bind to (genomic) DNA originating from maize inbred line B73. In certain embodiments, the sequence of the polynucleic acid or the complement or reverse complement thereof does not occur or is not present in maize inbred line B73.

In an aspect, the invention relates to a kit comprising one or more of the polynucleotides as described herein, such as one or more of the primers or probes as described herein. The skilled person will understand that the polynucleotides may be comprised for instance in a single receptacle, such as a single vial, or in separate receptacles, such as separate vials.

It will be understood that “specifically hybridizing” means that the polynucleic acid hybridises with the (molecular) marker allele (such as under stringent hybridisation conditions, as defined herein elsewhere), but does not (substantially) hybridise with a polynucleic acid not comprising the marker allele or is (substantially) incapable of being used as a PCR primer. By means of example, in a suitable readout, the hybridization signal with the marker allele or PCR amplification of the marker allele is at least 5 times, preferably at least 10 times stronger or more than the hybridisation signal with a non-marker allele, or any other sequence.

In an aspect, the invention relates to a set of primers or probes as described above, such as a set of allele-specific primers or probes. In certain embodiments, the set may further comprise a (common) forward or reverse primer (depending on whether the allele-specific primers are reverse or forward primers).

In an aspect, the invention relates to a kit comprising such polynucleic acids, such as primers (comprising forward (such as one or more allele-specific or alternatively common primers) and/or reverse primers (such as a common or alternatively one or more allele-specific primers)) and/or probes (such as one or more allele-specific probe). The kit may further comprise instructions for use.

It will be understood that in embodiments relating to a set of forward and reverse primers, only one of both primers (forward or reverse) may need to be capable of discriminating between a (molecular) marker allele of the invention and a non-marker allele, and hence may be unique. The other primer may or may not be capable of discriminating between a (molecular) marker allele of the invention and a non-marker allele, and hence may or may not be unique.

In an aspect, the invention relates to a vector comprising a (isolated) polynucleic acid according to the invention as described herein. In certain embodiments, the vector is a (plant) expression vector. In certain embodiments, the vector is an inducible (plant) expression vector. In certain embodiments, the expression is tissue- or organ-specific. In certain embodiments, the expression is developmentally specific. In certain embodiments, the expression is tissue- or organ-specific and developmentally specific.

In certain embodiments, the vector comprises any of SEQ ID NOs: 1-3, 5-7, 9-11, 13-15, 201, 203, 205, or 207, or the complement thereof, or the reverse complement thereof, or a (unique) fragment thereof, or a sequence which is at least 80%, preferably at least 85%, more preferably at least 90%, most preferably at least 95% identical to a sequence as set forth in any one or more of SEQ ID NOs: 1-3, 5-7, 9-11, 13-15, 201, 203, 205, or 207, or the complement thereof, or the reverse complement thereof, or a (unique) fragment thereof. In certain embodiments, the vector comprises a polynucleic acid encoding a protein of SEQ ID NOs: 4, 8, 12, 16, 202, 204, 206, or 208, or the complement thereof, or the reverse complement thereof, or a (unique) fragment thereof, or a sequence which is at least 80%, preferably at least 85%, more preferably at least 90%, most preferably at least 95% identical to a sequence as set forth in any one or more of SEQ ID NOs: 4, 8, 12, 16, 202, 204, 206, or 208, or the complement thereof, or the reverse complement thereof, or a (unique) fragment thereof. In certain embodiments, the vector comprises any of SEQ ID NOs: 1, 5, 9, 13, 201, 203, 205, or 207, preferably SEQ ID NO: 1 or 201, or the complement thereof, or the reverse complement thereof, or a (unique) fragment thereof, or a sequence which is at least 80%, preferably at least 85%, more preferably at least 90%, most preferably at least 95% identical to a sequence as set forth in any one or more of SEQ ID NOs:1, 5, 9, 13, 201, 203, 205, or 207, preferably SEQ ID NO: 1 or 201, or the complement thereof, or the reverse complement thereof, or a (unique) fragment thereof. In certain embodiments, the vector comprises a polynucleic acid encoding a protein of SEQ ID NOs: 202, 204, 206, 208, preferably SEQ ID NO: 202, or the complement thereof, or the reverse complement thereof, or a (unique) fragment thereof, or a sequence which is at least 80%, preferably at least 85%, more preferably at least 90%, most preferably at least 95% identical to a sequence as set forth in any one or more of SEQ ID NOs: 202, 204, 206, 208, preferably SEQ ID NO: 202, or the complement thereof, or the reverse complement thereof, or a (unique) fragment thereof. In certain embodiments, the vector comprises any of SEQ ID NOs: 2-3, 6-7, 10-11, 14-15, preferably SEQ ID NO: 2, or the complement thereof, or the reverse complement thereof, or a (unique) fragment thereof, or a sequence which is at least 80%, preferably at least 85%, more preferably at least 90%, most preferably at least 95% identical to a sequence as set forth in any one or more of SEQ ID NOs: 2-3, 6-7, 10-11, 14-15, preferably SEQ ID NO: 2, or the complement thereof, or the reverse complement thereof, or a (unique) fragment thereof. In certain embodiments, the vector comprises a polynucleic acid encoding a protein of SEQ ID NOs: 4, 8, 12, 16, preferably SEQ ID NO: 4, or the complement thereof, or the reverse complement thereof, or a (unique) fragment thereof, or a sequence which is at least 80%, preferably at least 85%, more preferably at least 90%, most preferably at least 95% identical to a sequence as set forth in any one or more of SEQ ID NOs: 4, 8, 12, 16, preferably SEQ ID NO: 4, or the complement thereof, or the reverse complement thereof, or a (unique) fragment thereof. Such polynucleic acids are suitable for identifying plants or plant parts as well as for generating plants as described herein elsewhere.

As used herein, a “vector” has its ordinary meaning in the art, and may for instance be a plasmid, a cosmid, a phage or an expression vector, a transformation vector, shuttle vector, or cloning vector; it may be double- or single-stranded, linear or circular; or it may transform a prokaryotic or eukaryotic host, either via integration into its genome or extrachromosomally. The nucleic acid according to the invention is preferably operatively linked in a vector with one or more regulatory sequences which allow the transcription, and, optionally, the expression, in a prokaryotic or eukaryotic host cell. A regulatory sequence—preferably DNA—may be homologous or heterologous to the nucleic acid according to the invention. For example, the nucleic acid is under the control of a suitable promoter or terminator. Suitable promoters may be promoters which are constitutively induced (example: 35S promoter from the “Cauliflower mosaic virus” (Odell et al., 1985); those promoters which are tissue-specific are especially suitable (example: Pollen-specific promoters, Chen et al. (2010), Zhao et al. (2006), or Twell et al. (1991)), or are development-specific (example: blossom-specific promoters). Suitable promoters may also be synthetic or chimeric promoters which do not occur in nature, are composed of multiple elements, and contain a minimal promoter, as well as—upstream of the minimum promoter—at least one cis-regulatory element which serves as a binding location for special transcription factors. Chimeric promoters may be designed according to the desired specifics and are induced or repressed via different factors. Examples of such promoters are found in Gurr & Rushton (2005) or Venter (2007). For example, a suitable terminator is the nos-terminator (Depicker et al., 1982). The vector may be introduced via conjugation, mobilization, biolistic transformation, agrobacteria-mediated transformation, transfection, transduction, vacuum infiltration, or electroporation. The vector may be a plasmid, a cosmid, a phage or an expression vector, a transformation vector, shuttle vector, or cloning vector; it may be double- or single-stranded, linear or circular. The vector may transform a prokaryotic or eukaryotic host, either via integration into its genome or extrachromosomally.

As used herein, the term “operatively linked” or “operably linked” means connected in a common nucleic acid molecule in such a manner that the connected elements are positioned and oriented relative to one another such that a transcription of the nucleic acid molecule may occur. A DNA which is operatively linked with a promoter is under the transcriptional control of this promoter.

In an aspect, the invention relates to the use of the polynucleic acid or vector according to the invention as described herein for generating a maize plant or plant part.

In certain embodiments, the vector is an expression vector. The nucleic acid is preferably operatively linked in a vector with one or more regulatory sequences which allow the transcription, and optionally the expression, in a prokaryotic or eukaryotic host cell. A regulatory sequence may be homologous or heterologous to the nucleic acid. For example, the nucleic acid is under the control of a suitable promoter or terminator. Suitable promoters may be promoters which are constitutively induced, for example, the 35S promoter from the “Cauliflower mosaic virus” (Odell et al., 1985. Identification of DNA sequences required for activity of the cauliflower mosaic virus 35S promoter.) Tissue-specific promoters, e.g. pollen-specific promoters as described in Chen et al. (2010. Molecular Biology Reports 37(2):737-744), Zhao et al. (2006. Planta 224(2): 405-412), or Twell et al. (1991. Genes & Development 5(3): 496-507), are particularly suitable, as are development-specific promoters, e.g. blossom-specific promoters. Suitable promoters may also be synthetic or chimeric promoters which do not occur in nature, and which are composed of multiple elements. Such synthetic or chimeric promoter may contain a minimal promoter, as well as at least one cis-regulatory element which serves as a binding location for special transcription factors. Chimeric promoters may be designed according to the desired specifics and can be induced or repressed via different factors. Examples of such promoters are found in Gurr & Rushton (2005. Trends in Biotechnology 23(6): 275-282) or Venter (2007. Trends in Plant Science: 12(3):, 118-124). For example, a suitable terminator is the nos-terminator (Depicker et al., 1982. Journal of Molecular and Applied Genetics 1(6): 561-573).

In certain embodiments, the vector is a conditional expression vector. In certain embodiments, the vector is a constitutive expression vector. In certain embodiments, the vector is a tissue-specific expression vector, such as a pollen-specific expression vector. In certain embodiments, the vector is an inducible expression vector. All such vectors are well-known in the art. Methods for preparation of the described vectors are commonplace to the person skilled in the art (Sambrook et al., 2001).

Also envisaged herein is a host cell, such as a plant cell, which comprises a nucleic acid as described herein, preferably an induction-promoting nucleic acid or a nucleic acid encoding a double-stranded RNA as described herein, or a vector as described herein. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

The host cell may be a prokaryotic (for example, bacterial) or eukaryotic cell (for example, a plant cell or a yeast cell). For example, the host cell may be an agrobacterium, such as Agrobacterium tumefaciens or Agrobacterium rhizogenes. Preferably, the host cell is a plant cell.

In an aspect, the invention relates to the use of the polynucleic acid or vector according to the invention as described herein for identifying a maize plant or plant part.

A nucleic acid described herein or a vector described herein may be introduced in a host cell via well-known methods, which may depend on the selected host cell, including, for example, conjugation, mobilization, biolistic transformation, agrobacteria-mediated transformation, transfection, transduction, vacuum infiltration, or electroporation. In particular, methods for introducing a nucleic acid or a vector in an agrobacterium cell are well-known to the skilled person and may include conjugation or electroporation methods. Also methods for introducing a nucleic acid or a vector into a plant cell are known (Sambrook et al., 2001) and may include diverse transformation methods such as biolistic transformation and agrobacterium-mediated transformation.

In particular embodiments, the present invention relates to a transgenic plant cell which comprises a nucleic acid as described herein, in particular an induction-promoting nucleic acid or a nucleic acid encoding a double-stranded RNA as described herein, as a transgene or a vector as described herein. In further embodiments, the present invention relates to a transgenic plant or a part thereof which comprises the transgenic plant cell.

For example, such a transgenic plant cell or transgenic plant is a plant cell or plant which is, preferably stably, transformed with a nucleic acid as described herein, in particular an induction-promoting nucleic acid or a nucleic acid encoding a double-stranded RNA as described herein, or a vector as described herein.

Preferably, the nucleic acid in the transgenic plant cell is operatively linked with one or more regulatory sequences which allow the transcription, and optionally the expression, in the plant cell. A regulatory sequence may be homologous or heterologous to the nucleic acid. The total structure made up of the nucleic acid according to the invention and the regulatory sequence(s) may then represent the transgene.

In an aspect, the invention relates to the use of one or more of the (molecular) marker (allele) described herein for identifying a plant or plant part having a fertility restorer (gene, locus, haplotype, genotype, or phenotype). In an aspect, the invention relates to the use of one or more of the (molecular) marker (allele) described herein which are able to detect at least one diagnostic marker allele for identifying a plant or plant part, such as having having a fertility restorer (gene, locus, haplotype, genotype, or phenotype). In an aspect, the invention relates to the detection of one or more of the (molecular) marker alleles described herein for identifying a plant or plant part having having a fertility restorer (gene, locus, haplotype, genotype, or phenotype).

In an aspect, the invention relates to a (maize) plant or plant part identified by the methods of the invention as described herein. In particular embodiments this includes plant material obtained from said plant or plant part.

In an aspect, the invention relates to a (maize) plant or plant part comprising one or more of the (molecular) markers (alleles), polynucleic acids, loci, or vectors of the invention as described herein.

In an aspect, the invention relates to a (maize) plant or plant part comprising one or more of the (molecular) marker (alleles) of Table 4 or 5.

In an aspect, the invention relates to a (maize) plant or plant part comprising one or more polynucleic acid of SEQ ID NO: 1 to 208. In an aspect, the invention relates to a (maize) plant or plant part comprising one or more polynucleic acid of SEQ ID NO: 17 to 200. In certain embodiments, the polynucleic acid of SEQ ID Nos: 17-200 corresponds to a polynucleic acid of a restorer of the invention. In certain embodiments, the polynucleic acid of SEQ ID Nos: 17-200 corresponds to a polynucleic acid of a non-restorer/maintainer of the invention. The skilled person will appreciate that the discrimination between restorer and non-restorer/maintainer can be made based on the identity of “n” in SEQ ID Nos 17-200, as also described herein elsewhere (such as for instance based on Table 4). In an aspect, the invention relates to a (maize) plant or plant part comprising one or more polynucleic acid of SEQ ID NO: 68 to 140. In an aspect, the invention relates to a (maize) plant or plant part comprising one or more polynucleic acid of SEQ ID NO: 70, 72, 76, 79, 86, 88, 92, 93, 99, 104, 105, 107, 108, 109, 115, and/or 134. In an aspect, the invention relates to a (maize) plant or plant part comprising one or more polynucleic acid of SEQ ID NO: 1, 5, 9, or 13, or a sequence which is at least 80%, preferably at least 85%, more preferably at least 90%, most preferably at least 95% identical to a sequence as set forth in any one or more of SEQ ID NOs: 1, 5, 9, or 13, or one or more of SEQ ID NOs: 2, 6, 10, and 14, or a sequence which is at least 80%, preferably at least 85%, more preferably at least 90%, most preferably at least 95% identical to a sequence as set forth in any one or more of SEQ ID NOs: 2, 6, 10, and 14. In an aspect, the invention relates to a (maize) plant or plant part comprising one or more polynucleic acid encoding Zm00001d043358, Zm00001d043352, Zm00001d043356, and Zm00001d043357, having respectively a coding sequence as set forth in any of SEQ ID NOs: 201, 203, 205, and 207, or a sequence which is at least 80%, preferably at least 85%, more preferably at least 90%, most preferably at least 95% identical to a sequence as set forth in any one or more of SEQ ID NOs: 201, 203, 205, and 207, or having respectively a coding sequence as set forth in any of SEQ ID NOs: 3, 7, 11, and 15, or a sequence which is at least 80%, preferably at least 85%, more preferably at least 90%, most preferably at least 95% identical to a sequence as set forth in any one or more of SEQ ID NOs: 3, 7, 11, and 15. In an aspect, the invention relates to a (maize) plant or plant part comprising one or more polypeptide having a sequence as set forth in any of SEQ ID NOs: 202, 204, 206, or 208, or a sequence which is at least 80%, preferably at least 85%, more preferably at least 90%, most preferably at least 95% identical to a sequence as set forth in any one or more of SEQ ID NOs: 202, 204, 206, or 208, or having a sequence as set forth in any of SEQ ID NOs: 4, 8, 12, or 16, or a sequence which is at least 80%, preferably at least 85%, more preferably at least 90%, most preferably at least 95% identical to a sequence as set forth in any one or more of SEQ ID NOs: 4, 8, 12, or 16. Preferably, the plant or plant part comprises a polynucleic acid encoding the polypeptide, which may be provided on a vector or may be genomically integrated.

In certain embodiments, the markers (alleles), polynucleic acids, or loci as defined herein are homozygous. Accordingly, in diploid plants the two alleles are identical (at least with respect to the particular marker (allele), polynucleic acid, or locus), in tetraploid plants the four alleles are identical, and in hexaploid plants the six alleles are identical with respect to the marker (allele), polynucleic acid, or locus. In certain embodiments, the marker (allele), polynucleic acid, or locus as defined herein is heterozygous. Accordingly, in diploid plants the two alleles are not identical, in tetraploid plants the four alleles are not identical (for instance only one, two, or three alleles comprise the specific marker (allele), polynucleic acid, or locus), and in hexaploid plants the six alleles are not identical with respect to the mutation or marker (for instance only one, two, three, four or five alleles comprise the specific marker (allele), polynucleic acid, or locus). Similar considerations apply in case of pseudopolyploid pants.

In an aspect, the invention relates to a method for generating a (maize) plant or plant part comprising introducing in (the genome of) said plant or plant part a polypeptide, polynucleic acid, locus (allele), or (molecular) marker (allele) of the invention as defined herein, or a (functional) fragment thereof. Preferably, introduction into the plant or plant part is genomic introduction. In certain embodiments however, introduction is non-genomic introduction, such as episomal introduction. In certain embodiments, introduction is achieved by means of a vector, as is known in the art and as also described herein elsewhere.

In an aspect, the invention relates to a method for generating a (maize) plant or plant part comprising introducing in (the genome of) said plant or plant part one or more polynucleic acid of SEQ ID NO: 1 to 208. In an aspect, the invention relates to a method for generating a (maize) plant or plant part comprising introducing in (the genome of) said plant or plant part one or more polynucleic acid of SEQ ID NO: 17 to 200. In certain embodiments, the polynucleic acid of SEQ ID Nos: 17-200 corresponds to a polynucleic acid of a restorer of the invention. In certain embodiments, the polynucleic acid of SEQ ID Nos: 17-200 corresponds to a polynucleic acid of a non-restorer/maintainer of the invention. The skilled person will appreciate that the discrimination between restorer and non-restorer/maintainer can be made based on the identity of “n” in SEQ ID Nos 17-200, as also described herein elsewhere (such as for instance based on Table 4). In an aspect, the invention relates to a method for generating a (maize) plant or plant part comprising introducing in (the genome of) said plant or plant part one or more polynucleic acid of SEQ ID NO: 68 to 140. In an aspect, the invention relates to a method for generating a (maize) plant or plant part comprising introducing in (the genome of) said plant or plant part one or more polynucleic acid of SEQ ID NO: 70, 72, 76, 79, 86, 88, 92, 93, 99, 104, 105, 107, 108, 109, 115, and/or 134. In an aspect, the invention relates to a method for generating a (maize) plant or plant part comprising introducing in (the genome of) said plant or plant part one or more polynucleic acid of SEQ ID NO: 1, 5, 9, or 13, or a sequence which is at least 80%, preferably at least 85%, more preferably at least 90%, most preferably at least 95% identical to a sequence as set forth in any one or more of SEQ ID NOs: 1, 5, 9, or 13, or one or more of SEQ ID NOs: 2, 6, 10, and 14, or a sequence which is at least 80%, preferably at least 85%, more preferably at least 90%, most preferably at least 95% identical to a sequence as set forth in any one or more of SEQ ID NOs: 2, 6, 10, and 14. In an aspect, the invention relates to a method for generating a (maize) plant or plant part comprising introducing in (the genome of) said plant or plant part one or more polynucleic acid encoding Zm00001d043358, Zm00001d043352, Zm00001d043356, and Zm00001d043357, having respectively a coding sequence as set forth in any of SEQ ID NOs: 201, 203, 205, and 207, or a sequence which is at least 80%, preferably at least 85%, more preferably at least 90%, most preferably at least 95% identical to a sequence as set forth in any one or more of SEQ ID NOs: 201, 203, 205, and 207, or having respectively a coding sequence as set forth in any of SEQ ID NOs: 3, 7, 11, and 15, or a sequence which is at least 80%, preferably at least 85%, more preferably at least 90%, most preferably at least 95% identical to a sequence as set forth in any one or more of SEQ ID NOs: 3, 7, 11, and 15. In an aspect, the invention relates to a method for generating a (maize) plant or plant part comprising introducing in said plant or plant part one or more polypeptide having a sequence as set forth in any of SEQ ID NOs: 202, 204, 206, or 208, or a sequence which is at least 80%, preferably at least 85%, more preferably at least 90%, most preferably at least 95% identical to a sequence as set forth in any one or more of SEQ ID NOs: 202, 204, 206, or 208, or having a sequence as set forth in any of SEQ ID NOs: 4, 8, 12, or 16, or a sequence which is at least 80%, preferably at least 85%, more preferably at least 90%, most preferably at least 95% identical to a sequence as set forth in any one or more of SEQ ID NOs: 4, 8, 12, or 16. Preferably, the plant or plant part comprises a polynucleic acid encoding the polypeptide, which may be provided on a vector or may be genomically integrated. In an aspect, the invention relates to a method for generating a (maize) plant or plant part comprising introducing in (the genome of) said plant or plant part one or more polynucleic acid encoding a polypeptide having a sequence as set forth in any of SEQ ID NOs: 202, 204, 206, or 208, or a sequence which is at least 80%, preferably at least 85%, more preferably at least 90%, most preferably at least 95% identical to a sequence as set forth in any one or more of SEQ ID NOs: 202, 204, 206, or 208, or having a sequence as set forth in any of SEQ ID NOs: 4, 8, 12, or 16, or a sequence which is at least 80%, preferably at least 85%, more preferably at least 90%, most preferably at least 95% identical to a sequence as set forth in any one or more of SEQ ID NOs: 4, 8, 12, or 16.

The skilled person will understand that preferably genomic sequences are introduced.

In certain embodiments, introducing (into the genome) as referred to herein comprises transgenesis.

In certain embodiments, introducing (into the genome) as referred to herein comprises transformation.

In certain embodiments, introducing (into the genome) as referred to herein comprises recombination, such as homologous recombination.

In certain embodiments, introducing (into the genome) as referred to herein comprises mutagenesis.

In certain embodiments, introducing (into the genome) as referred to herein comprises introgression. In certain embodiments, introducing into the genome as referred to herein does not comprise introgression.

In certain embodiments, introducing into the genome as referred to herein comprises introducing into the genome in a plant part. In certain embodiments, the plant part is a plant organ. In certain embodiments, the plant part is a plant tissue. In certain embodiments, the plant part is a plant cell. In certain embodiments, the plant part is a protoplast.

In certain embodiments, introducing into the genome as referred to herein comprises introducing into the genome in vitro. In certain embodiments, introducing into the genome as referred to herein comprises introducing into the genome in vivo.

In certain embodiments, the method for generating a maize plant or plant part comprises transforming a plant or plant part, preferably a plant cell, more preferably a protoplast, with a polynucleic acid as described herein elsewhere, and optionally regenerating a plant from said plant cell, preferably protoplast.

In certain embodiments, the transformed plant or plant part does not endogenously comprise the polynucleic acid according to the invention as described herein.

In certain embodiments, the transformed plant or plant part does not endogenously comprise the one or more molecular marker (alleles) according to the invention as described herein.

In certain embodiments, the methods for obtaining or generating plants or plant parts as described herein according to the invention involve or comprise transgenesis and/or gene editing, such as including CRISPR/Cas, TALEN, ZFN, meganucleases; (induced) mutagenesis, which may or may not be random mutagenesis, such as TILLING.

In certain embodiments, the methods for obtaining plants or plant parts as described herein according to the invention do not involve or comprise transgenesis, gene editing, and/or mutagenesis.

In certain embodiments, the methods for obtaining plants or plant parts as described herein according to the invention involve, comprise or consist of breeding and/or selection.

In certain embodiments, the methods for obtaining plants or plant parts as described herein according to the invention do not involve, comprise or consist of breeding.

In an aspect, the invention relates to a maize plant or plant part obtained or obtainable by the methods according to the invention as described herein, such as the methods for identifying a maize plant or plant part or the methods for generating a maize plant or plant part. The invention also relates to the progeny of such plants.

In an aspect, the invention relates to a maize plant or plant part comprising a polynucleic acid according to the invention as described herein. In certain embodiments, the polynucleic acid allele is homozygous. In certain embodiments, the polynucleic acid allele is heterozygous.

In an aspect, the invention relates to a maize plant or plant part comprising any one or more molecular marker (allele) according to the invention as described herein. In certain embodiments, the molecular marker (allele) is homozygous. In certain embodiments, the molecular marker (allele) allele is heterozygous.

In certain embodiments, the maize plant is not a maize variety. In certain embodiments, the plant is not exclusively obtained by means of an essentially biological process. In certain embodiments, the plant is obtained by a method which contains at least one step other than crossing, i.e. the screening for the presence of a polynucleotide as described herein.

As described herein elsewhere, in certain embodiments such (maize) plant or plant part does not comprise endogenously the recited polynucleic acids.

In certain embodiments, the maize plant or plant part is transgenic, gene-edited, or mutagenized. In certain embodiments, the maize plant or plant part is transgenic, gene-edited, or mutagenized in order to comprise the one or more molecular marker (allele), or one or more of the polynucleic acids according to the invention as described herein.

In an aspect, the invention relates to a method for generating a (maize) plant or plant part, comprising (a) providing a first (maize) plant according to the invention or identified according to the invention or generated according to the invention, (b) crossing said first (maize) plant with a second maize plant having cytoplasmic male sterility; and optionally (d) harvesting said (maize) plant part from the progeny.

In an aspect, the invention relates to a method for generating a (maize) plant or plant part, comprising (a) providing a first (maize) plant according to the invention or identified according to the invention or generated according to the invention, (b) crossing said first (maize) plant with a second maize plant having cytoplasmic male sterility; and optionally (d) harvesting said (maize) plant part from the progeny, wherein said first plant is a restorer (preferably a restorer of the present invention).

In an aspect, the invention relates to a method for generating a (maize) plant or plant part, comprising (a) providing a first (maize) plant according to the invention or identified according to the invention or generated according to the invention, (b) crossing said first (maize) plant with a second maize plant having cytoplasmic male sterility; and optionally (d) harvesting said (maize) plant part from the progeny, wherein said second plant is a restorer (preferably a restorer of the present invention).

In an aspect, the invention relates to a method for generating a (maize) plant or plant part, comprising (a) providing a first (maize) plant according to the invention or identified according to the invention or generated according to the invention, (b) crossing said first (maize) plant with a second maize plant having cytoplasmic male sterility; and optionally (d) harvesting said (maize) plant part from the progeny, wherein said first plant is a not a restorer (preferably not a restorer of the present invention).

In an aspect, the invention relates to a method for generating a (maize) plant or plant part, comprising (a) providing a first (maize) plant according to the invention or identified according to the invention or generated according to the invention, (b) crossing said first (maize) plant with a second maize plant having cytoplasmic male sterility; and optionally (d) harvesting said (maize) plant part from the progeny, wherein said second plant is a not a restorer (preferably not a restorer of the present invention).

In an aspect, the invention relates to a method for generating a (maize) plant or plant part, comprising (a) providing a first (maize) plant according to the invention or identified according to the invention or generated according to the invention, (b) crossing said first (maize) plant with a second maize plant having cytoplasmic male sterility; and optionally (d) harvesting said (maize) plant part from the progeny, wherein said first plant is a restorer (preferably a restorer of the present invention) and wherein said second plant is not a restorer (preferably not a restorer of the present invention).

In an aspect, the invention relates to a method for generating a (maize) plant or plant part, comprising (a) providing a first (maize) plant according to the invention or identified according to the invention or generated according to the invention, (b) crossing said first (maize) plant with a second maize plant having cytoplasmic male sterility; and optionally (d) harvesting said (maize) plant part from the progeny, wherein said first plant is a not a restorer (preferably not a restorer of the present invention) and wherein said second plant is a restorer (preferably a restorer of the present invention).

In an aspect, the invention relates to a method for generating a (maize) plant or plant part, comprising crossing a first (maize) plant with a second maize plant, and selecting offspring comprising any one or more of the loci, haplotypes, polynucleic acids, marker(s) (allele(s)), polymorphisms, or SNPs as described herein.

In an aspect, the invention relates to a method for generating a (maize) plant or plant part, comprising crossing a first (maize) plant with a second maize plant, and selecting offspring lacking any one or more of the loci, haplotypes, polynucleic acids, marker(s) (allele(s)), polymorphisms, or SNPs as described herein.

In an aspect, the invention relates to a method for generating a (maize) plant or plant part, comprising crossing a first (maize) plant with a second maize plant, and selecting offspring comprising any one or more of the restorer (-associated) loci, haplotypes, polynucleic acids, marker(s) (allele(s)), polymorphisms, or SNPs as described herein.

In an aspect, the invention relates to a method for generating a (maize) plant or plant part, comprising crossing a first (maize) plant with a second maize plant, and selecting offspring lacking any one or more of the restorer (-associated) loci, haplotypes, polynucleic acids, marker(s) (allele(s)), polymorphisms, or SNPs as described herein.

In an aspect, the invention relates to a method for generating a (maize) plant or plant part, comprising crossing a first (maize) plant with a second maize plant, and selecting offspring comprising any one or more of the maintainer (-associated) loci, haplotypes, polynucleic acids, marker(s) (allele(s)), polymorphisms, or SNPs as described herein.

In an aspect, the invention relates to a method for generating a (maize) plant or plant part, comprising crossing a first (maize) plant with a second maize plant, and selecting offspring lacking any one or more of the maintainer (-associated) loci, haplotypes, polynucleic acids, marker(s) (allele(s)), polymorphisms, or SNPs as described herein.

Preferably, the first or second plant is a cytoplasmic male sterile plant.

The restorer or maintainer loci, haplotypes, polynucleic acids, marker(s) (allele(s)), polymorphisms, or SNPs may comprise the respective markers, polymorphisms or SNPs as listed in Tables 4 or 5.

In certain embodiments, plants or plant parts are selected which do not comprise the restorer locus of the invention.

In certain embodiments, plants or plant parts are selected which comprise the restorer locus of the invention.

The aspects and embodiments of the invention are further supported by the following non-limiting examples. The following examples, including the experiments conducted and the results achieved, are provided for illustrative purposes only and are not constructed as limiting the present invention.

### EXAMPLES

### Example 1: Identification of Maize Restorer Genotype on Chromosome 3

In the last years, a higher fraction of restoring lines in the female breeding pool 4 has been observed for CMSC, but only part of these lines carries the restorer RF4.

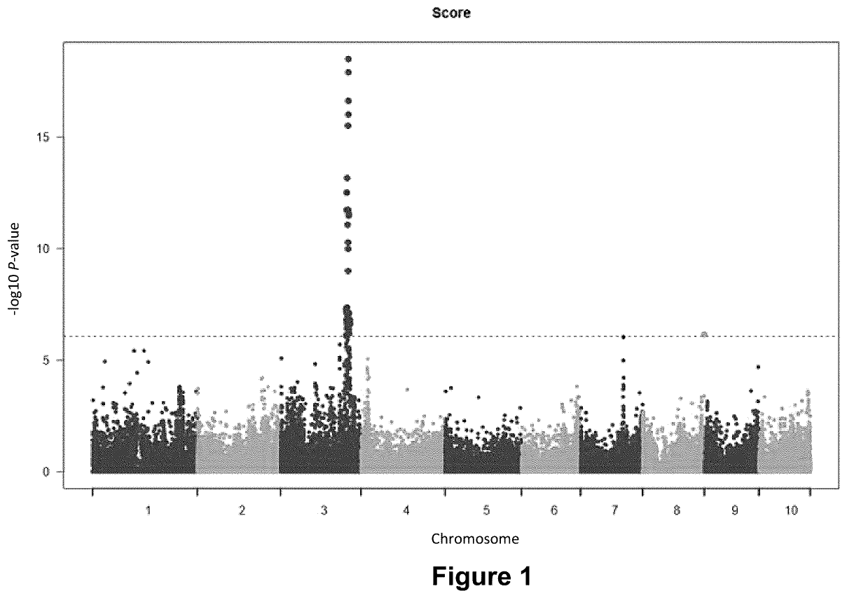

The present inventors tested 374 corn lines with phenotype of the pool 4 for the presence of RF4 und the respective restoration phenotype (Table 1). The data show that one or more other restorers seems to be present in the pool. A GWAS analysis using all pool 4 lines not carrying RF4 confirmed this assumption and showed a clear hit on chromosome 3 (FIG. 1). The new restorer is named RF-03-01.

TABLE 1
Analysis of presence/absence of RF4 restorer (RF4+/rf4−)

| Haplotype | Maintainer | Restorer |
|---|---|---|
| RF4+ | 1 | 45 |
| rf4− | 174 | 93 |

Analysis of 600k data of 143 restoring and not restoring pool4 lines (all RF4 free) showed a genomic region of 0.25 Mb linked to restoration. Within the analyzed material, only two haplotypes were present, one of them explaining restoration. The haplotypes contain 64 polymorphic 600k markers (Table 4: identifiers 52, 53, 55, 57-71, 73-75, 78-88, 90, 94-124) which are in high linkage disequilibrium (LD). 14% of all pool 4 lines (including RF4 carriers) carry the RF-03-01 restorer haplotype.

TABLE 2
Haplotype analysis

| Haplotype | Maintainer | Restorer |
|---|---|---|
| A | 100 | 22 |
| B | 0 | 21 |

The restoring fraction of haplotype A can only be explained by further minor restorers which could not be identified until now.

The region on chromosome 3 contains 6 genes according the AGPv4 annotation https://www.maizegdb.org/genome/assembly/Zm-B73-REFERENCE-GRAMENE-4.0), 4 of them being expressed in pollen, one being involved in mitochondrial organization and thus representing the most prominent candidate gene (Zm00001d043358). All four genes are polymorphic between the two haplotypes.

TABLE 3
List of candidate genes

| | | SEQ ID NO: | | | | | |
|---|---|---|---|---|---|---|---|
| | | Restorer haplotype | | | Reference B73 | | |
| | | genomic | | | genomic | | |
| Gene | Function | DNA | cDNA | protein | DNA | CDNA | protein |
| Zm00001d043358 | mitochondrion | 1 | 201 | 202 | 2 | 3 | 4 |
| | organization | | | | | | |
| Zm00001d043352 | rRNA | 5 | 203 | 204 | 6 | 7 | 8 |
| | processing | | | | | | |
| Zm00001d043356 | aromatic | 9 | 205 | 206 | 10 | 11 | 12 |
| | amino acid | | | | | | |
| | family | | | | | | |
| | biosynthetic | | | | | | |
| | process | | | | | | |
| Zm00001d043357 | Plastocyanin- | 13 | 207 | 208 | 14 | 15 | 16 |
| | like, | | | | | | |
| | Electron | | | | | | |
| | carrier | | | | | | |

16 KASP markers (Table 4: identifier 54, 56, 60, 63, 70, 72, 76, 77, 83, 88, 89, 91, 92, 93, 99, and 118) were developed which can be used to detect the haplotype, partly by conversion of 600k markers, partly by using new SNPs within the candidate genes.

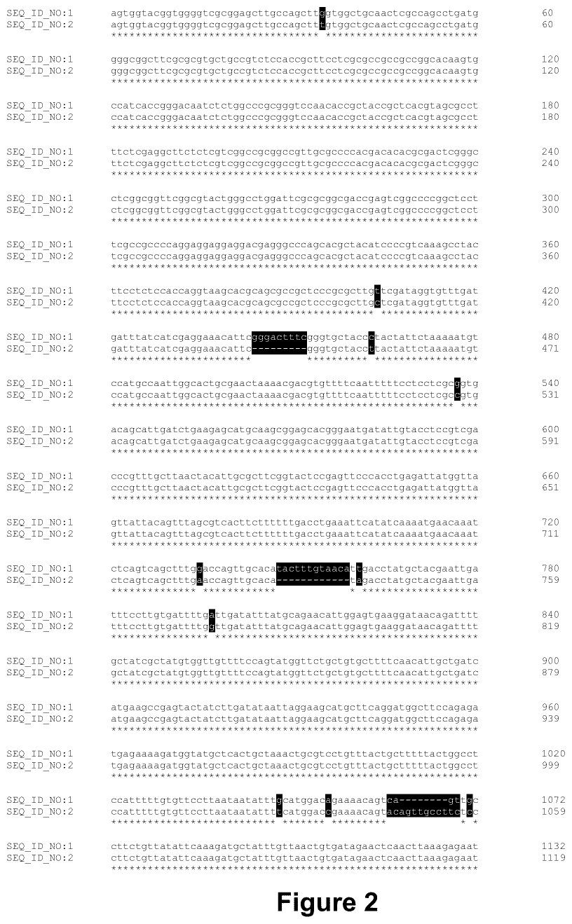

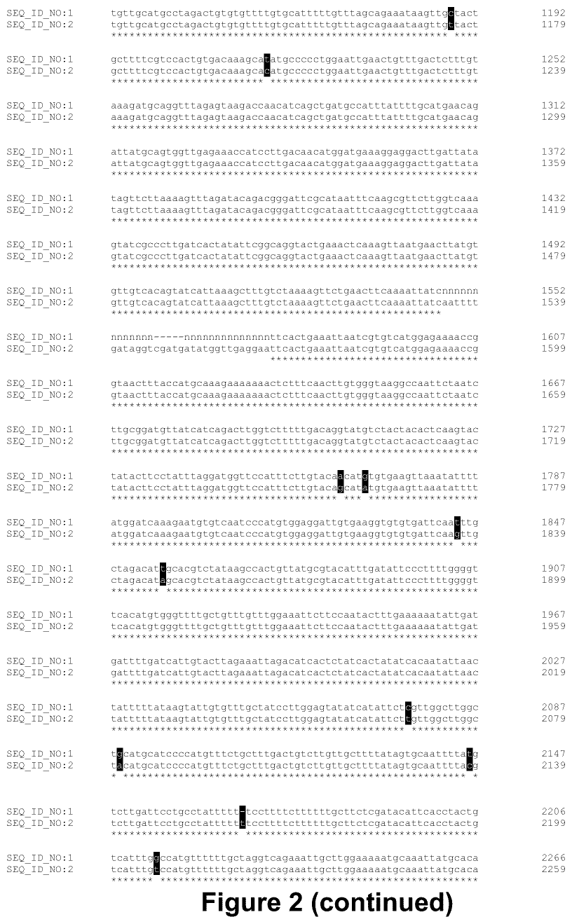

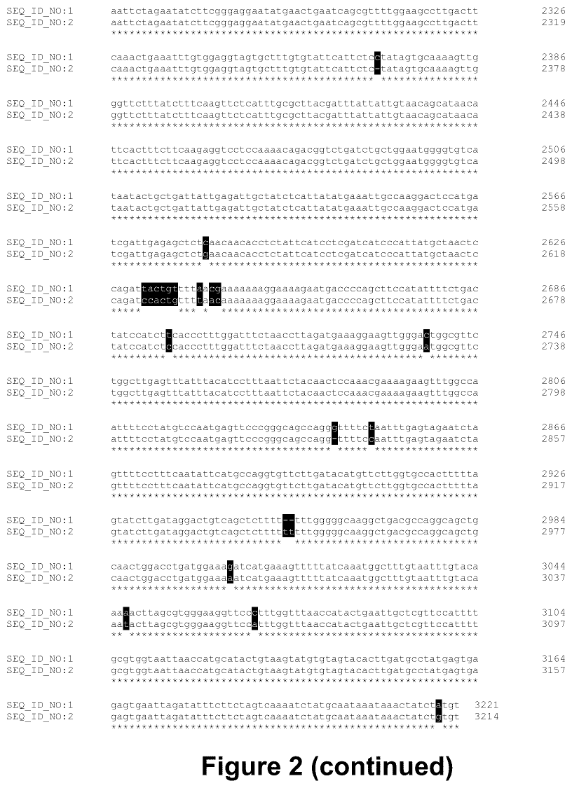

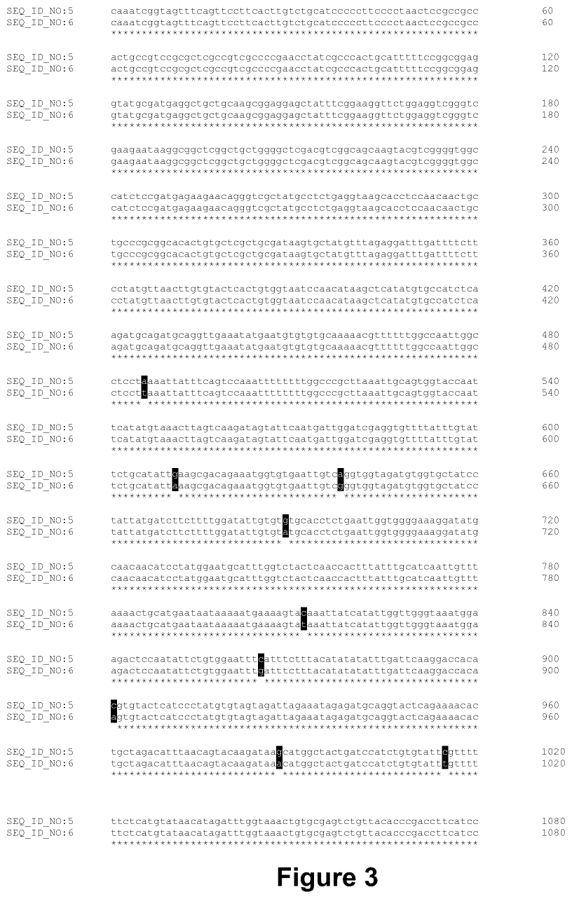

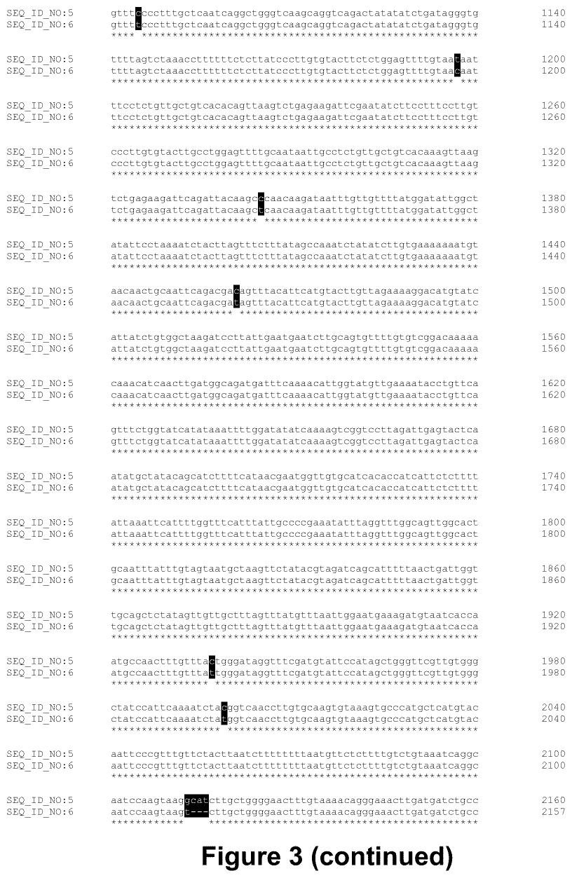

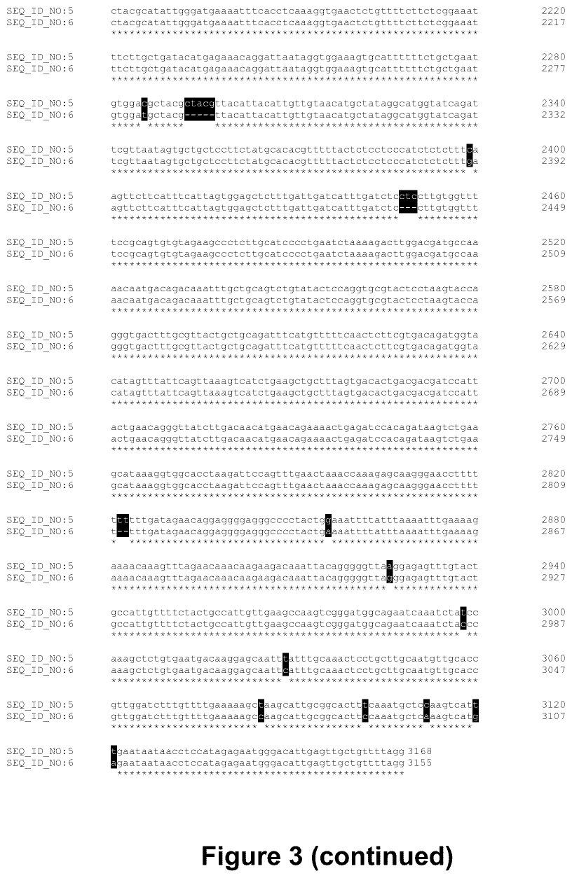

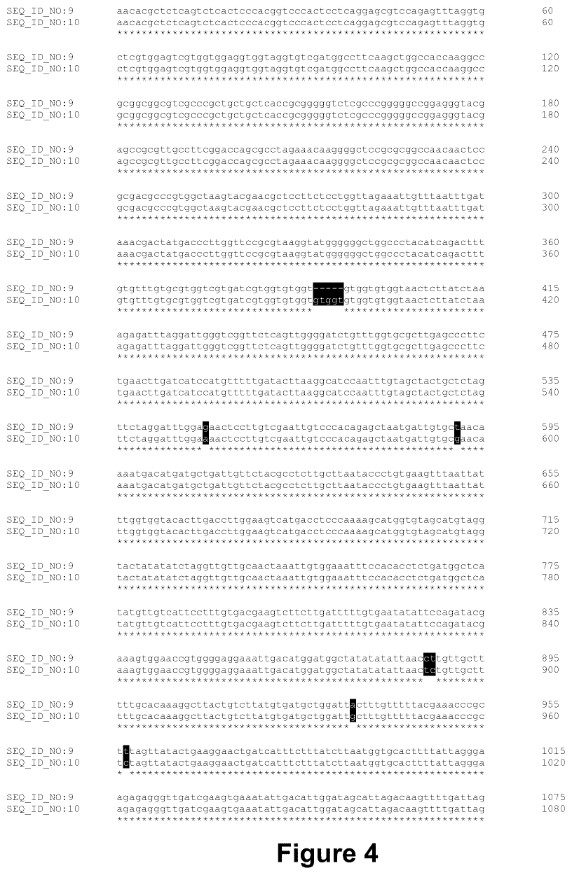

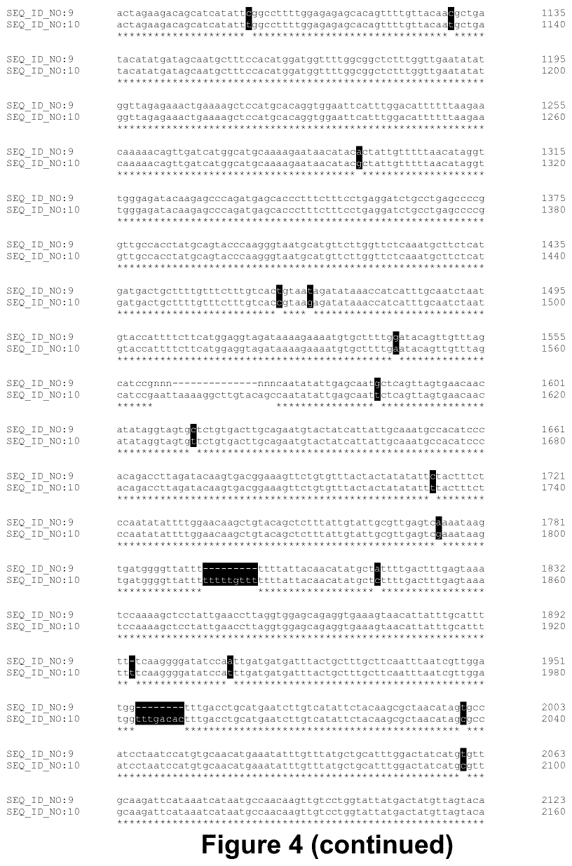

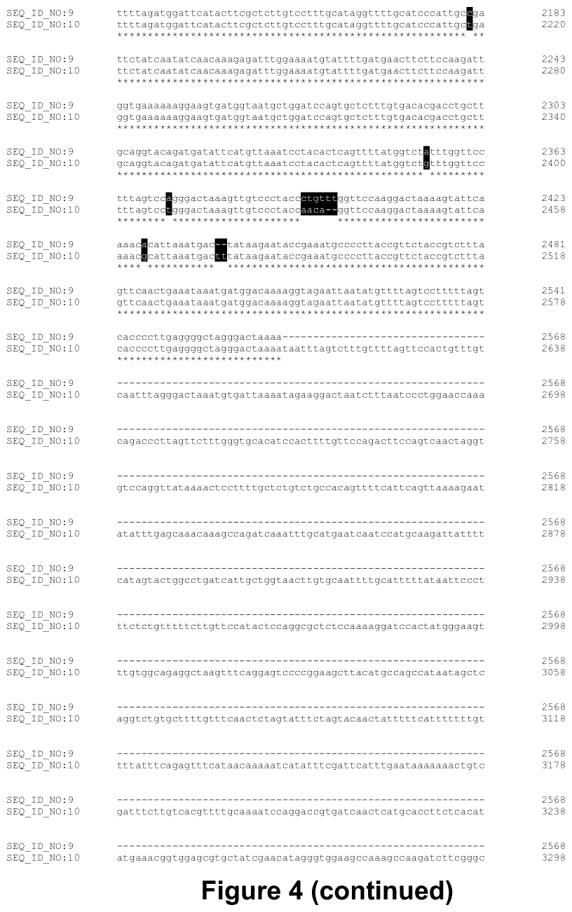

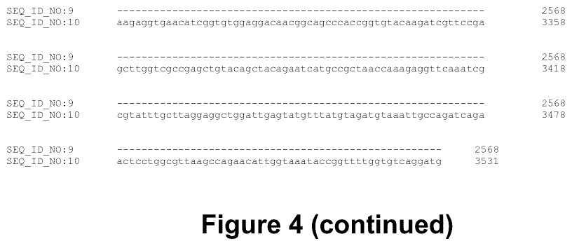

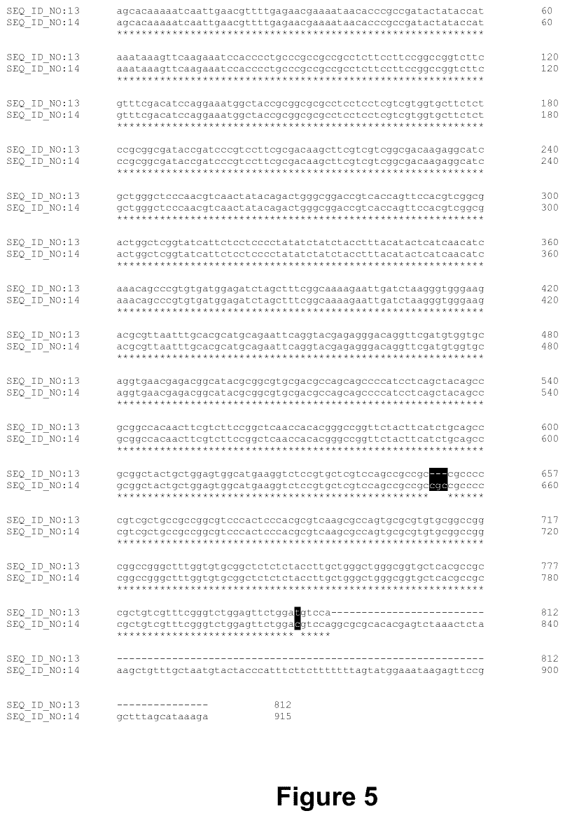

Using these markers in combination with known RF4 markers, it is possible to detect the most important restorers in pool 4 and to decide on the most reasonable conversion strategy for a given line in the process of development of hybrid corn lines. Generally all markers as listed in Table 4 can be used for this purpose. Further, FIGS. 2-5 show sequence alignments of the genomic DNA of the candidate genes derived from the RF-03-01 restorer genotype and from a reference genotype (1B73). Highlighted in black with white letters are polymorphisms which are additionally suitable to detect undesired restorer genotype. With regard to the list of markers according to Table 4 markers with the identifiers 52-124 showed to be 100% associated to the restorer locus on chromosome 3. Therefore, the region from position 197453646 to 197698278 referenced to B73 AGPv4 is most suitable as target site for marker-associated identification of RF-03-01 restorer genotype. Markers with identifiers 1-51 and 125-184 corresponding to regions from position 195629901 to 196989408 and from position 197708137 to 198023573 can also be used for identification, because the underlying polymorphisms can be found in most genotypes (major alleles), however it is not 100% linked.

RF-03-01 is also present in the flint pool used as male, although its impact to restoration is lower than in pool 4. Anyhow, also in this pool a good knowledge of restorer genes should be achieved, because restoring male lines are important for the usefulness of cms. In case only RF-03-01 is present, but not RF4, the restoration may be too weak or may fail in some environments, because this restorer is not as stable as RF4. Therefore, a good genotyping approach in addition to phenotyping is important to secure production.

TABLE 4
List of markers; marker sequences can be found in sequence
listing under respective SEQ ID NO as indicated in last column.
Further, in the sequence listing under identifier <223> it is
defined where in the marker sequence the polymorphism is located
(number following the @ gives the position in the sequence).
Position ″n″ thus corresponds to the polymorphism capable
of discriminating between restorer and non-restorer/maintainer.

| | | | | | SEQ |
|---|---|---|---|---|---|
| | | Position | polymorphism | | ID |
| Identifier | Marker name | AGPv4 | maintainer | restorer | NO: |
| 1 | ma0016fm86 | 195629901 | ade | gua | 17 |
| 2 | ma0016fn05 | 195639694 | thy | cy | 18 |
| 3 | ma0016fn29 | 195677799 | gua | ade | 19 |
| 4 | ma0016fn27 | 195678356 | cyt | gua | 20 |
| 5 | ma0016fn16 | 195680790 | gua | ade | 21 |
| 6 | ma0004tk22 | 195732936 | gua | ade | 22 |
| 7 | ma0004tk05 | 195733916 | cyt | ade | 23 |
| 8 | ma0016fn55 | 195783701 | cyt | thy | 24 |
| 9 | ma0016fp29 | 196070086 | cyt | thy | 25 |
| 10 | ma0004tm29 | 196198736 | ade | gua | 26 |
| 11 | ma0004tm38 | 196244714 | thy | cy | 27 |
| 12 | ma0016fq46 | 196653746 | cyt | thy | 28 |
| 13 | ma0000ba98 | 196693501 | cyt | ade | 29 |
| 14 | ma0000na46 | 196702811 | ade | gua | 30 |
| 15 | ma0001gr36 | 196704008 | ade | gua | 31 |
| 16 | ma0000kr37 | 196704096 | thy | cyt | 32 |
| 17 | ma0001gr37 | 196704169 | thy | cyt | 33 |
| 18 | ma0001gr41 | 196704290 | thy | cyt | 34 |
| 19 | ma0016fq61 | 196705470 | thy | ade | 35 |
| 20 | ma0022xj91 | 196706697 | gua | ade | 36 |
| 21 | ma0016fq64 | 196706755 | cyt | ade | 37 |
| 22 | ma0001jh28 | 196707190 | ade | gua | 38 |
| 23 | ma0004tn40 | 196707415 | thy | ade | 39 |
| 24 | ma0004tn32 | 196707997 | gua | gua | 40 |
| 25 | ma0016fq88 | 196773893 | cyt | thy | 41 |
| 26 | ma0004tn60 | 196774122 | cyt | thy | 42 |
| 27 | ma0016fq93 | 196774333 | gua | ade | 43 |
| 28 | ma0016fq89 | 196774502 | gua | ade | 44 |
| 29 | ma0016fq94 | 196774823 | cyt | thy | 45 |
| 30 | ma0004tn62 | 196774965 | cyt | gua | 46 |
| 31 | ma0004tn31 | 196775596 | gua | ade | 47 |
| 32 | ma0016fq50 | 196776600 | ade | gua | 48 |
| 33 | ma0004tn30 | 196776877 | cyt | thy | 49 |
| 34 | ma0000xk01 | 196840068 | cyt | ade | 50 |
| 35 | ma0011cv03 | 196840815 | gua | cyt | 51 |
| 36 | ma0016fq79 | 196841649 | gua | cyt | 52 |
| 37 | ma0004tn51 | 196841990 | cyt | ade | 53 |
| 38 | ma0004tn53 | 196843002 | cyt | gua | 54 |
| 39 | ma0000cm74 | 196843335 | cyt | gua | 55 |
| 40 | ma0000jt04 | 196844084 | cyt | thy | 56 |
| 41 | ma0016fq81 | 196851451 | thy | cyt | 57 |
| 42 | ma0016fq76 | 196853534 | gua | cyt | 58 |
| 43 | ma0016fq97 | 196853762 | cyt | thy | 59 |
| 44 | ma0004tn72 | 196880373 | cyt | thy | 60 |
| 45 | ma0004tn96 | 196985856 | cyt | ade | 61 |
| 46 | ma0016fr46 | 196985883 | cyt | thy | 62 |
| 47 | ma0011rt36 | 196987284 | cyt | thy | 63 |
| 48 | ma0022vg84 | 196989025 | gua | thy | 64 |
| 49 | ma0001hr21 | 196989252 | gua | ade | 65 |
| 50 | ma0012wu17 | 196989377 | gua | ade | 66 |
| 51 | ma0001hr20 | 196989408 | ade | gua | 67 |
| 52 | ma0000sa77 | 197453646 | ade | gua | 68 |
| 53 | ma0004tp73 | 197453708 | ade | gua | 69 |
| 54 | ma61758s03 | 197453708 | thy | cyt | 70 |
| 55 | ma0000qw65 | 197454448 | gua | ade | 71 |
| 56 | ma61758s02 | 197454630 | thy | cyt | 72 |
| 57 | ma0016ft38 | 197454657 | thy | cyt | 73 |
| 58 | ma0016ft37 | 197454744 | thy | gua | 74 |
| 59 | ma0000wv70 | 197454780 | cyt | gua | 75 |
| 60 | ma0004tp85 | 197454833 | ade | gua | 76 |
| 61 | ma0012sk12 | 197455007 | cyt | thy | 77 |
| 62 | ma0022mm17 | 197455034 | thy | cyt | 78 |
| 63 | ma0004tp78 | 197456922 | thy | cyt | 79 |
| 64 | ma0022mm15 | 197457134 | ade | gua | 80 |
| 65 | ma0004tp79 | 197457214 | cyt | thy | 81 |
| 66 | ma0004tp82 | 197457351 | gua | ade | 82 |
| 67 | ma0016ft40 | 197457603 | gua | ade | 83 |
| 68 | ma0016ft28 | 197459188 | thy | cyt | 84 |
| 69 | ma0004tq11 | 197487965 | gua | ade | 85 |
| 70 | ma0004tq10 | 197488751 | cyt | thy | 86 |
| 71 | ma0012aq25 | 197489067 | gua | ade | 87 |
| 72 | ma61757s01 | 197524489 | gua | thy | 88 |
| 73 | ma0016ft65 | 197524855 | cyt | thy | 89 |
| 74 | ma0004tq05 | 197525193 | gua | ade | 90 |
| 75 | ma0004tq06 | 197525365 | gua | thy | 91 |
| 76 | ma61757s03 | 197525625 | thy | cyt | 92 |
| 77 | ma61757s04 | 197525990 | cyt | thy | 93 |
| 78 | ma0004tq13 | 197526621 | ade | gua | 94 |
| 79 | ma0016ft76 | 197526690 | gua | ade | 95 |
| 80 | ma0016ft75 | 197527582 | gua | ade | 96 |
| 81 | ma0022xj97 | 197527682 | cyt | gua | 97 |
| 82 | ma0004tq07 | 197528652 | cyt | thy | 98 |
| 83 | ma0016ft73 | 197556227 | gua | ade | 99 |
| 84 | ma0004tq16 | 197609686 | gua | thy | 100 |
| 85 | ma0016ft77 | 197609730 | ade | gua | 10 |
| 86 | ma0016ft86 | 197611692 | ade | gua | 102 |
| 87 | ma0010yr95 | 197611832 | gua | ade | 103 |
| 88 | ma0011dy20 | 197611894 | gua | ade | 104 |
| 89 | ma61755s04 | 197613135 | ade | cyt | 105 |
| 90 | ma0016ft80 | 197613658 | ade | cyt | 106 |
| 91 | ma61755s03 | 197613658 | thy | gua | 107 |
| 92 | ma61755s02 | 197615089 | gua | ade | 108 |
| 93 | ma61755s01 | 197615461 | cyt | thy | 109 |
| 94 | ma0016ft81 | 197631560 | ade | gua | 110 |
| 95 | ma0016ft78 | 197631688 | gua | ade | 111 |
| 96 | ma0016ft83 | 197632590 | gua | ade | 112 |
| 97 | ma0004tq35 | 197632706 | ade | gua | 113 |
| 98 | ma0004tq36 | 197633370 | thy | cyt | 114 |
| 99 | ma0004tq32 | 197633860 | gua | ade | 115 |
| 100 | ma0022xj98 | 197638778 | thy | cyt | 116 |
| 101 | ma0004tq24 | 197638949 | thy | cyt | 117 |
| 102 | ma0004tq26 | 197639380 | gua | ade | 118 |
| 103 | ma0004tq30 | 197652023 | cyt | thy | 119 |
| 104 | ma0016ft97 | 197652478 | ade | gua | 120 |
| 105 | ma0004tq33 | 197653125 | gua | thy | 121 |
| 106 | ma0016ft92 | 197654542 | cyt | gua | 122 |
| 107 | ma0004tq39 | 197687270 | cyt | thy | 123 |
| 108 | ma0022mm26 | 197687524 | cyt | thy | 124 |
| 109 | ma0016fu15 | 197688212 | gua | cyt | 125 |
| 110 | ma0010yr98 | 197688445 | thy | cyt | 126 |
| 111 | ma0022mm25 | 197688492 | thy | cyt | 127 |
| 112 | ma0016ft98 | 197692991 | ade | gua | 128 |
| 113 | ma0004tq38 | 197692996 | thy | cyt | 129 |
| 114 | ma0004tq54 | 197694265 | thy | cyt | 130 |
| 115 | ma0016fu20 | 197695368 | cyt | thy | 131 |
| 116 | ma0016fu17 | 197695591 | ade | gua | 132 |
| 117 | ma0004tq57 | 197695857 | thy | cyt | 133 |
| 118 | ma0016fu11 | 197696192 | thy | ade | 134 |
| 119 | ma0016fu10 | 197696732 | gua | thy | 135 |
| 120 | ma0016fu09 | 197696762 | gua | ade | 136 |
| 121 | ma0004tq43 | 197697327 | gua | ade | 137 |
| 122 | ma0016fu01 | 197697514 | cyt | thy | 138 |
| 123 | ma0004tq42 | 197698249 | gua | ade | 139 |
| 124 | ma0016fu05 | 197698278 | gua | ade | 140 |
| 125 | ma0016ft99 | 197708137 | thy | cyt | 141 |
| 126 | ma0016fu07 | 197708334 | thy | ade | 142 |
| 127 | ma0010yt02 | 197758073 | thy | gua | 143 |
| 128 | ma0000gu42 | 197760175 | gua | ade | 144 |
| 129 | ma52981s02 | 197761254 | cyt | ade | 145 |
| 130 | ma52981s01 | 197761305 | ade | cyt | 146 |
| 131 | ma0004tq83 | 197776540 | thy | cyt | 147 |
| 132 | ma0004tq66 | 197777549 | ade | gua | 148 |
| 133 | ma0016fu32 | 197777618 | thy | ade | 149 |
| 134 | ma0011zk10 | 197778110 | ade | gua | 150 |
| 135 | ma0004tq62 | 197781849 | cyt | ade | 151 |
| 136 | ma0004tq68 | 197781961 | gua | ade | 152 |
| 137 | ma0004tq78 | 197784696 | ade | gua | 153 |
| 138 | ma0004tq59 | 197785166 | cyt | thy | 154 |
| 139 | ma0022mm29 | 197785270 | cyt | gua | 155 |
| 140 | ma53009s01 | 197786148 | gua | ade | 156 |
| 141 | ma0016fu41 | 197786155 | cyt | gua | 157 |
| 142 | ma0016fu35 | 197787768 | cyt | thy | 158 |
| 143 | ma0016fu36 | 197806056 | gua | ade | 159 |
| 144 | ma0004tq69 | 197806483 | cyt | thy | 160 |
| 145 | ma0016fu28 | 197812595 | gua | ade | 161 |
| 146 | ma0016fu26 | 197813589 | thy | cyt | 162 |
| 147 | ma0010yt03 | 197814082 | gua | ade | 163 |
| 148 | ma0016fu45 | 197840802 | thy | cyt | 164 |
| 149 | ma0022mm28 | 197840951 | gua | ade | 165 |
| 150 | ma0022xj99 | 197855989 | cyt | thy | 166 |
| 151 | ma0016fu43 | 197859323 | gua | cyt | 167 |
| 152 | ma0004tq81 | 197860711 | ade | cyt | 168 |
| 153 | ma0016fu29 | 197861373 | gua | ade | 169 |
| 154 | ma0022mm31 | 197895272 | ade | gua | 170 |
| 155 | ma0010yt04 | 197902823 | gua | ade | 171 |
| 156 | ma0004tr07 | 197902855 | cyt | thy | 172 |
| 157 | ma0012fz92 | 197902923 | cyt | thy | 173 |
| 158 | ma0001ac17 | 197903119 | cyt | ade | 174 |
| 159 | ma0012pg35 | 197903264 | thy | cyt | 175 |
| 160 | ma0000en22 | 197903302 | gua | cyt | 176 |
| 161 | ma0000en21 | 197903372 | cyt | thy | 177 |
| 162 | ma0016fu62 | 197903475 | cyt | thy | 178 |
| 163 | ma0004tq90 | 197903587 | gua | thy | 179 |
| 164 | ma08364s01 | 197903629 | gua | ade | 180 |
| 165 | ma0011zt60 | 197903716 | thy | cyt | 181 |
| 166 | ma0004tq98 | 197903816 | thy | gua | 182 |
| 167 | ma0016fu52 | 197904016 | thy | thy | 183 |
| 168 | ma0016fu55 | 197904655 | cyt | ade | 184 |
| 169 | ma0011dy21 | 197906673 | ade | gua | 185 |
| 170 | ma0004tq88 | 197907566 | gua | ade | 186 |
| 171 | ma0022xk01 | 197907617 | ade | cyt | 187 |
| 172 | ma0016fu57 | 197907653 | thy | cyt | 188 |
| 173 | ma0010yt06 | 197907842 | thy | cyt | 189 |
| 174 | ma0001dg29 | 197909824 | ade | ade | 190 |
| 175 | ma0016fu59 | 197948546 | ade | gua | 191 |
| 176 | ma0016fu58 | 197948580 | cyt | thy | 192 |
| 177 | ma0004tr05 | 197948690 | cyt | ade | 193 |
| 178 | ma0004tr10 | 197948831 | gua | gua | 194 |
| 179 | ma0004tr03 | 197948879 | ade | gua | 195 |
| 180 | ma0004tq84 | 197973632 | cyt | cyt | 196 |
| 181 | ma0016fu56 | 197974493 | ade | gua | 197 |
| 182 | ma0011jn34 | 197994208 | thy | gua | 198 |
| 183 | ma0016fu80 | 198023432 | thy | cyt | 199 |
| 184 | ma0004tr23 | 198023573 | cyt | gua | 200 |

TABLE 5
List of markers based in FIGS. 2-5.
Nucleotide positions are indicated
for the respective SEQ ID NO.

| | | | | Posi- | | |
|---|---|---|---|---|---|---|
| | | | | tion | | |
| | | Posi- | | B73 | | |
| | SEQ | tion | SEQ | (AGPv4) = | polymorphism | |
| Identi- | ID | re- | ID | main- | re- | main- |
| fier | NO: | storer | NO: | tainer | storer | tainer |
| 1 | 1 | 35 | 2 | 35 | g | t |
| 2 | 1 | 404 | 2 | 404 | t | c |
| 3 | 1 | 444- | 2 | 443- | Insertion | cg |
| | | 452 | | 444 | of | |
| | | | | | gggactttc | |
| | | | | | between | |
| | | | | | cg | |
| 4 | 1 | 463 | 2 | 454 | c | t |
| 5 | 1 | 537 | 2 | 528 | g | c |
| 6 | 1 | 735 | 2 | 726 | g | a |
| 7 | 1 | 748- | 2 | 738- | Insertion | at |
| | | 759 | | 739 | of | |
| | | | | | tactttg | |
| | | | | | taaca | |
| | | | | | between | |
| | | | | | at | |
| 8 | 1 | 761 | 2 | 740 | t | a |
| 9 | 1 | 797 | 2 | 776 | a | g |
| 10 | 1 | 1048 | 2 | 1027 | g | t |
| 11 | 1 | 1056 | 2 | 1035 | a | c |
| 12 | 1 | 1065- | 2 | 1045 | Deletion | a |
| | | 1066 | | | of | |
| | | | | | a between | |
| | | | | | tc | |
| 13 | 1 | 1072- | 2 | 1053- | Deletion | cttc |
| | | 1073 | | 1059 | of | tcc |
| | | | | | cttctcc | |
| | | | | | between | |
| | | | | | cc | |
| 14 | 1 | 1071 | 2 | 1058 | g | c |
| 15 | 1 | 1188 | 2 | 1175 | c | t |
| 16 | 1 | 1218 | 2 | 1208 | t | c |
| 17 | 1 | 1765 | 2 | 1757 | a | g |
| 18 | 1 | 1769 | 2 | 1761 | g | a |
| 19 | 1 | 1844 | 2 | 1836 | t | g |
| 20 | 1 | 1856 | 2 | 1848 | t | a |
| 21 | 1 | 2076 | 2 | 2068 | c | t |
| 22 | 1 | 2089 | 2 | 2081 | g | a |
| 23 | 1 | 2146 | 2 | 2138 | t | c |
| 24 | 1 | 2168- | 2 | 2161 | Deletion | t |
| | | 2169 | | | of t | |
| | | | | | between | |
| | | | | | tt | |
| 25 | 1 | 2214 | 2 | 2207 | g | t |
| 26 | 1 | 2370 | 2 | 2362- | Insertion | ct |
| | | | | 2363 | of | |
| | | | | | c between | |
| | | | | | ct | |
| 27 | 1 | 2582 | 2 | 2574 | c | g |
| 28 | 1 | 2632- | 2 | 2624- | tactgt | cca |
| | | 2637 | | 2629 | | ctg |
| 29 | 1 | 2641 | 2 | 2633 | a | t |
| 30 | 1 | 2643- | 2 | 2635- | cg | ac |
| | | 2644 | | 2636 | | |
| 31 | 1 | 2696 | 2 | 2688 | t | c |
| 32 | 1 | 2738 | 2 | 2730 | c | a |
| 33 | 1 | 2843 | 2 | 2834- | Insertion | gt |
| | | | | 2835 | of | |
| | | | | | g between | |
| | | | | | gt | |
| 34 | 1 | 2849 | 2 | 2840 | t | c |
| 35 | 1 | 2954- | 2 | 2946- | Deletion | tt |
| | | 2955 | | 2947 | of | |
| | | | | | tt | |
| | | | | | between | |
| | | | | | tt | |
| 36 | 1 | 3004 | 2 | 2997 | g | a |
| 37 | 1 | 3047 | 2 | 3040 | a | t |
| 38 | 1 | 3068 | 2 | 3061 | c | a |
| 39 | 1 | 3218 | 2 | 3211 | a | g |
| 40 | 5 | 486 | 6 | 486 | a | t |
| 41 | 5 | 611 | 6 | 611 | g | a |
| 42 | 5 | 638 | 6 | 638 | a | g |
| 43 | 5 | 689 | 6 | 689 | g | a |
| 44 | 5 | 812 | 6 | 812 | c | t |
| 45 | 5 | 865 | 6 | 865 | c | g |
| 46 | 5 | 901 | 6 | 901 | c | a |
| 47 | 5 | 988 | 6 | 988 | g | a |
| 48 | 5 | 1015 | 6 | 1015 | c | t |
| 49 | 5 | 1085 | 6 | 1085 | c | t |
| 50 | 5 | 1197 | 6 | 1197 | t | c |
| 51 | 5 | 1345 | 6 | 1345 | c | t |
| 52 | 5 | 1461 | 6 | 1461 | c | t |
| 53 | 5 | 1937 | 6 | 1937 | c | t |
| 54 | 5 | 1999 | 6 | 1999 | c | t |
| 55 | 5 | 2113- | 6 | 2112- | Insertion | gt |
| | | 2115 | | 2113 | of | |
| | | | | | gca | |
| | | | | | between | |
| | | | | | gt | |
| 56 | 5 | 2286 | 6 | 2283 | c | t |
| 57 | 5 | 2293- | 6 | 2289- | Insertion | gt |
| | | 2297 | | 2290 | of | |
| | | | | | ctacg | |
| | | | | | between | |
| | | | | | gt | |
| 58 | 5 | 2399 | 6 | 2391 | c | g |
| 59 | 5 | 2448- | 6 | 2439- | Insertion | cc |
| | | 2450 | | 2440 | of | |
| | | | | | ctc | |
| | | | | | between | |
| | | | | | cc | |
| 60 | 5 | 2822- | 6 | 2810- | Insertion | tt |
| | | 2823 | | 2811 | of tt | |
| | | | | | between | |
| | | | | | tt | |
| 61 | 5 | 2856 | 6 | 2843 | g | a |
| 62 | 5 | 2926 | 6 | 2913 | a | g |
| 63 | 5 | 2998 | 6 | 2985 | t | c |
| 64 | 5 | 3029 | 6 | 3016 | t | c |
| 65 | 5 | 3085 | 6 | 3072 | t | c |
| 66 | 5 | 3102 | 6 | 3089 | t | c |
| 67 | 5 | 3112 | 6 | 3099 | c | a |
| 68 | 5 | 3120 | 6 | 3107 | t | g |
| 69 | 5 | 3168 | 6 | 3155 | t | a |
| 70 | 9 | 392- | 10 | 393- | Deletion | gtggt |
| | | 393 | | 397 | of | |
| | | | | | gtggt | |
| | | | | | between | |
| | | | | | tg | |
| 71 | 9 | 550 | 10 | 555 | g | a |
| 72 | 9 | 591 | 10 | 596 | t | g |
| 73 | 9 | 886- | 10 | 891- | ct | tc |
| | | 887 | | 892 | | |
| 74 | 9 | 934 | 10 | 939 | a | g |
| 75 | 9 | 957 | 10 | 962 | t | c |
| 76 | 9 | 1097 | 10 | 1102 | c | t |
| 77 | 9 | 1130 | 10 | 1135 | c | t |
| 78 | 9 | 1295 | 10 | 1300 | a | g |
| 79 | 9 | 1462 | 10 | 1467 | t | c |
| 80 | 9 | 1467 | 10 | 1472 | t | g |
| 81 | 9 | 1541 | 10 | 1546 | g | a |
| 82 | 9 | 1584 | 10 | 1603 | g | t |
| 83 | 9 | 1614 | 10 | 1633 | c | t |
| 84 | 9 | 1713 | 10 | 1732 | c | t |
| 85 | 9 | 1774 | 10 | 1793 | a | g |
| 86 | 9 | 1795- | 10 | 1815- | Deletion | tttt |
| | | 1796 | | 1823 | of | tgttt |
| | | | | | ttttt | |
| | | | | | gttt | |
| | | | | | between | |
| | | | | | tt | |
| 87 | 9 | 1815 | 10 | 1843 | a | c |
| 88 | 9 | 1894- | 10 | 1923 | Deletion | t |
| | | 1895 | | | of t | |
| | | | | | between | |
| | | | | | tt | |
| 89 | 9 | 1910 | 10 | 1939 | a | t |
| 90 | 9 | 1954- | 10 | 1984- | Deletion | tttg |
| | | 1955 | | 1991 | of | acac |
| | | | | | tttga | |
| | | | | | cac | |
| | | | | | between | |
| | | | | | gt | |
| 91 | 9 | 2000 | 10 | 2037 | t | c |
| 92 | 9 | 2060 | 10 | 2097 | t | c |
| 93 | 9 | 2181 | 10 | 2218 | c | t |
| 94 | 9 | 2354 | 10 | 2391 | a | g |
| 95 | 9 | 2372 | 10 | 2409 | a | t |
| 96 | 9 | 2394- | 10 | 2431- | Substi- | aaca |
| | | 2399 | | 2434 | tution/ | |
| | | | | | Insertion | |
| | | | | | ctgttt | |
| 97 | 9 | 2428 | 10 | 2463 | a | g |
| 98 | 9 | 2439- | 10 | 2475- | Deletion | tt |
| | | 2440 | | 2476 | of tt | |
| | | | | | between | |
| | | | | | ct | |
| 99 | 13 | 651- | 14 | 652- | Deletion | cgc |
| | | 652 | | 654 | of | |
| | | | | | cgc | |
| | | | | | between | |
| | | | | | cc | |
| 100 | 13 | 807 | 14 | 810 | t | c |

1. A method for identifying a maize plant or plant part, comprising screening for the presence of a haplotype associated with a cytoplasmic male sterility fertility restorer locus on chromosome 3 RF-03-01.

2. The method according to claim 1, wherein said locus comprises or is comprised in a region on chromosome 3 corresponding to positions 195629901 to 198023573 of B73 AGPv4, preferably corresponding to positions 197453646 to 197698278 of B73 AGPv4 or a fragment thereof.

3. The method according to claim 1, wherein said locus comprises one or more of molecular marker(s) alleles of Table 4 or Table 5.

4. The method according to claim 1, wherein said locus comprises a polynucleic acid comprising one or more of SEQ ID NOs: 1, 5, 9, and 13, or a sequence which is at least 80%, preferably at least 85%, more preferably at least 90%, most preferably at least 95% identical to a sequence as set forth in any one or more of SEQ ID NOs: 1, 5, 9, and 13; or one or more of SEQ ID NOs: 2, 6, 10, and 14, or a sequence which is at least 80%, preferably at least 85%, more preferably at least 90%, most preferably at least 95% identical to a sequence as set forth in any one or more of SEQ ID NOs: 2, 6, 10, and 14.

5. The method according to claim 1, comprising screening for the presence of any one or more of SEQ ID NOs: 17 to 200.

6. The method according to claim 1, comprising screening for the presence of any one or more of SEQ ID NOs: 1, 5, 9, and 13, or a sequence which is at least 80%, preferably at least 85%, more preferably at least 90%, most preferably at least 95% identical to a sequence as set forth in any one or more of SEQ ID NOs: 1, 5, 9, and 13; or one or more of SEQ ID NOs: 2, 6, 10, and 14, or a sequence which is at least 80%, preferably at least 85%, more preferably at least 90%, most preferably at least 95% identical to a sequence as set forth in any one or more of SEQ ID NOs: 2, 6, 10, and 14.

7. The method according to claim 4, wherein the sequence which is at least 80%, preferably at least 85%, more preferably at least 90%, most preferably at least 95% identical to a sequence as set forth in any one or more of SEQ ID NOs: 1, 5, 9, or 13 comprises on or more, preferably all, of the respective associated restorer polymorphism(s) as listed in Table 5.

8. The method according to claim 4, wherein the sequence which is at least 80%, preferably at least 85%, more preferably at least 90%, most preferably at least 95% identical to a sequence as set forth in any one or more of SEQ ID NOs: 2, 6, 10, or 14 comprises on or more, preferably all, of the respective associated maintainer polymorphism(s) as listed in Table 5.

9. The method according to claim 1, which is a method for discriminating between a maize plant or plant part having said haplotype associated with cytoplasmic male sterility fertility restorer locus on chromosome 3 (RF-03-01) and a maize plant lacking said haplotype associated with cytoplasmic male sterility fertility restorer locus on chromosome 3 RF-03-01.

10. The method according to claim 1, wherein said maize plant or plant part is identified as having said haplotype associated with cytoplasmic male sterility fertility restorer locus on chromosome 3 RF-03-01 if said haplotype associated with cytoplasmic male sterility fertility restorer locus on chromosome 3 RF-03-01 is detected.

11. The method according to claim 1, wherein said maize plant or plant part is identified as lacking said haplotype associated with cytoplasmic male sterility fertility restorer locus on chromosome 3 RF-03-01 if said haplotype associated with cytoplasmic male sterility fertility restorer locus on chromosome 3 RF-03-01 is not detected.

12. The method according to claim 1, wherein said maize plant or plant part is identified as having said haplotype associated with cytoplasmic male sterility fertility restorer locus on chromosome 3 RF-03-01 if one or more of restorer molecular marker(s) allele(s) of Table 4 or Table 5 is detected.

13. The method according to claim 1, wherein said maize plant or plant part is identified as having said haplotype associated with cytoplasmic male sterility fertility restorer locus on chromosome 3 RF-03-01 if one or more of restorer molecular marker(s) allele(s) of Table 4 or Table 5 is not detected or if one or more of maintainer molecular marker(s) allele(s) of Table 4 or Table 5 is detected.

14. The method according to claim 1, wherein said maize plant or plant part is identified as having said haplotype associated with cytoplasmic male sterility fertility restorer locus on chromosome 3 RF-03-01 if one or more of SEQ ID NOs: 1, 5, 9, and 13, or a sequence which is at least 80%, preferably at least 85%, more preferably at least 90%, most preferably at least 95% identical to a sequence as set forth in any one or more of SEQ ID NOs: 1, 5, 9, and 13 is detected.

15. The method according to claim 1, wherein said maize plant or plant part is identified as having said haplotype associated with cytoplasmic male sterility fertility restorer locus on chromosome 3 RF-03-01 if one or more of SEQ ID NOs: 1, 5, 9, and 13, or a sequence which is at least 80%, preferably at least 85%, more preferably at least 90%, most preferably at least 95% identical to a sequence as set forth in any one or more of SEQ ID NOs: 1, 5, 9, and 13 is not detected or if one or more of SEQ ID NOs: 2, 6, 10, and 14, or a sequence which is at least 80%, preferably at least 85%, more preferably at least 90%, most preferably at least 95% identical to a sequence as set forth in any one or more of SEQ ID NOs: 2, 6, 10, and 14 is detected.

16. The method according to claim 1, wherein said maize plant or plant part is identified as having said haplotype associated with cytoplasmic male sterility fertility restorer locus on chromosome 3 RF-03-01 if one or more of SEQ ID NOs: 17 to 200 having the restorer SNP is detected.

17. The method according to claim 1, wherein said maize plant or plant part is identified as having said haplotype associated with cytoplasmic male sterility fertility restorer locus on chromosome 3 RF-03-01 if one or more of SEQ ID NOs: 17 to 200 having the restorer SNP is not detected, or if one or more of SEQ ID NOs: 17 to 200 having the maintainer SNP is detected.

18. An isolated polynucleic acid comprising one or more molecular marker allele of Table 4 or Table 5, or the complement or reverse complement of said polynucleic acid.

19. An isolated polynucleic acid comprising or consisting of one or more of SEQ ID NOs: 1, 5, 9, and 13, or a fragment thereof, or a sequence which is at least 80%, preferably at least 85%, more preferably at least 90%, most preferably at least 95% identical to a sequence as set forth in any one or more of SEQ ID NOs: 1, 5, 9, and 13, or a fragment thereof, or one or more of SEQ ID NOs: 2, 6, 10, and 14, or a fragment thereof, or a sequence which is at least 80%, preferably at least 85%, more preferably at least 90%, most preferably at least 95% identical to a sequence as set forth in any one or more of SEQ ID NOs: 2, 6, 10, and 14, or a fragment thereof.

20. The isolated polynucleotide according to claim 19, wherein the sequence which is at least 80%, preferably at least 85%, more preferably at least 90%, most preferably at least 95% identical to a sequence as set forth in any one or more of SEQ ID NOs: 1, 5, 9, or 13 comprises on or more, preferably all, of the respective associated restorer polymorphism(s) as listed in Table 5.

21. The isolated polynucleotide according to claim 19, wherein the sequence which is at least 80%, preferably at least 85%, more preferably at least 90%, most preferably at least 95% identical to a sequence as set forth in any one or more of SEQ ID NOs: 2, 6, 10, or 14 comprises on or more, preferably all, of the respective associated maintainer polymorphism(s) as listed in Table 5.

22. An isolated polynucleic acid comprising at least 15 contiguous nucleotides comprised in a region corresponding to a region flanked by any of the indicated 5′ and 3′ positions of the table below and comprising the nucleotide corresponding to the position of the indicated SNP referenced to maize chromosome 3, B73 AGPv4 or the complement, or reverse complement of said polynucleic acid.

| 5' (−100 nt) | 5' (−50 nt) | SNP | 3' (+50 nt) | 3' (+100 nt) |
|---|---|---|---|---|
| 195629801 | 195629851 | 195629901 | 195629951 | 195630001 |
| 195639594 | 195639644 | 195639694 | 195639744 | 195639794 |
| 195677699 | 195677749 | 195677799 | 195677849 | 195677899 |
| 195678256 | 195678306 | 195678356 | 195678406 | 195678456 |
| 195680690 | 195680740 | 195680790 | 195680840 | 195680890 |
| 195732836 | 195732886 | 195732936 | 195732986 | 195733036 |
| 195733816 | 195733866 | 195733916 | 195733966 | 195734016 |
| 195783601 | 195783651 | 195783701 | 195783751 | 195783801 |
| 196069986 | 196070036 | 196070086 | 196070136 | 196070186 |
| 196198636 | 196198686 | 196198736 | 196198786 | 196198836 |
| 196244614 | 196244664 | 196244714 | 196244764 | 196244814 |
| 196653646 | 196653696 | 196653746 | 196653796 | 196653846 |
| 196693401 | 196693451 | 196693501 | 196693551 | 196693601 |
| 196702711 | 196702761 | 196702811 | 196702861 | 196702911 |
| 196703908 | 196703958 | 196704008 | 196704058 | 196704108 |
| 196703996 | 196704046 | 196704096 | 196704146 | 196704196 |
| 196704069 | 196704119 | 196704169 | 196704219 | 196704269 |
| 196704190 | 196704240 | 196704290 | 196704340 | 196704390 |
| 196705370 | 196705420 | 196705470 | 196705520 | 196705570 |
| 196706597 | 196706647 | 196706697 | 196706747 | 196706797 |
| 196706655 | 196706705 | 196706755 | 196706805 | 196706855 |
| 196707090 | 196707140 | 196707190 | 196707240 | 196707290 |
| 196707315 | 196707365 | 196707415 | 196707465 | 196707515 |
| 196707897 | 196707947 | 196707997 | 196708047 | 196708097 |
| 196773793 | 196773843 | 196773893 | 196773943 | 196773993 |
| 196774022 | 196774072 | 196774122 | 196774172 | 196774222 |
| 196774233 | 196774283 | 196774333 | 196774383 | 196774433 |
| 196774402 | 196774452 | 196774502 | 196774552 | 196774602 |
| 196774723 | 196774773 | 196774823 | 196774873 | 196774923 |
| 196774865 | 196774915 | 196774965 | 196775015 | 196775065 |
| 196775496 | 196775546 | 196775596 | 196775646 | 196775696 |
| 196776500 | 196776550 | 196776600 | 196776650 | 196776700 |
| 196776777 | 196776827 | 196776877 | 196776927 | 196776977 |
| 196839968 | 196840018 | 196840068 | 196840118 | 196840168 |
| 196840715 | 196840765 | 196840815 | 196840865 | 196840915 |
| 196841549 | 196841599 | 196841649 | 196841699 | 196841749 |
| 196841890 | 196841940 | 196841990 | 196842040 | 196842090 |
| 196842902 | 196842952 | 196843002 | 196843052 | 196843102 |
| 196843235 | 196843285 | 196843335 | 196843385 | 196843435 |
| 196843984 | 196844034 | 196844084 | 196844134 | 196844184 |
| 196851351 | 196851401 | 196851451 | 196851501 | 196851551 |
| 196853434 | 196853484 | 196853534 | 196853584 | 196853634 |
| 196853662 | 196853712 | 196853762 | 196853812 | 196853862 |
| 196880273 | 196880323 | 196880373 | 196880423 | 196880473 |
| 196985756 | 196985806 | 196985856 | 196985906 | 196985956 |
| 196985783 | 196985833 | 196985883 | 196985933 | 196985983 |
| 196987184 | 196987234 | 196987284 | 196987334 | 196987384 |
| 196988925 | 196988975 | 196989025 | 196989075 | 196989125 |
| 196989152 | 196989202 | 196989252 | 196989302 | 196989352 |
| 196989277 | 196989327 | 196989377 | 196989427 | 196989477 |
| 196989308 | 196989358 | 196989408 | 196989458 | 196989508 |
| 197453546 | 197453596 | 197453646 | 197453696 | 197453746 |
| 197453608 | 197453658 | 197453708 | 197453758 | 197453808 |
| 197454348 | 197454398 | 197454448 | 197454498 | 197454548 |
| 197454530 | 197454580 | 197454630 | 197454680 | 197454730 |
| 197454557 | 197454607 | 197454657 | 197454707 | 197454757 |
| 197454644 | 197454694 | 197454744 | 197454794 | 197454844 |
| 197454680 | 197454730 | 197454780 | 197454830 | 197454880 |
| 197454733 | 197454783 | 197454833 | 197454883 | 197454933 |
| 197454907 | 197454957 | 197455007 | 197455057 | 197455107 |
| 197454934 | 197454984 | 197455034 | 197455084 | 197455134 |
| 197456822 | 197456872 | 197456922 | 197456972 | 197457022 |
| 197457034 | 197457084 | 197457134 | 197457184 | 197457234 |
| 197457114 | 197457164 | 197457214 | 197457264 | 197457314 |
| 197457251 | 197457301 | 197457351 | 197457401 | 197457451 |
| 197457503 | 197457553 | 197457603 | 197457653 | 197457703 |
| 197459088 | 197459138 | 197459188 | 197459238 | 197459288 |
| 197487865 | 197487915 | 197487965 | 197488015 | 197488065 |
| 197488651 | 197488701 | 197488751 | 197488801 | 197488851 |
| 197488967 | 197489017 | 197489067 | 197489117 | 197489167 |
| 197524389 | 197524439 | 197524489 | 197524539 | 197524589 |
| 197524755 | 197524805 | 197524855 | 197524905 | 197524955 |
| 197525093 | 197525143 | 197525193 | 197525243 | 197525293 |
| 197525265 | 197525315 | 197525365 | 197525415 | 197525465 |
| 197525525 | 197525575 | 197525625 | 197525675 | 197525725 |
| 197525890 | 197525940 | 197525990 | 197526040 | 197526090 |
| 197526521 | 197526571 | 197526621 | 197526671 | 197526721 |
| 197526590 | 197526640 | 197526690 | 197526740 | 197526790 |
| 197527482 | 197527532 | 197527582 | 197527632 | 197527682 |
| 197527582 | 197527632 | 197527682 | 197527732 | 197527782 |
| 197528552 | 197528602 | 197528652 | 197528702 | 197528752 |
| 197556127 | 197556177 | 197556227 | 197556277 | 197556327 |
| 197609586 | 197609636 | 197609686 | 197609736 | 197609786 |
| 197609630 | 197609680 | 197609730 | 197609780 | 197609830 |
| 197611592 | 197611642 | 197611692 | 197611742 | 197611792 |
| 197611732 | 197611782 | 197611832 | 197611882 | 197611932 |
| 197611794 | 197611844 | 197611894 | 197611944 | 197611994 |
| 197613035 | 197613085 | 197613135 | 197613185 | 197613235 |
| 197613558 | 197613608 | 197613658 | 197613708 | 197613758 |
| 197614989 | 197615039 | 197615089 | 197615139 | 197615189 |
| 197615361 | 197615411 | 197615461 | 197615511 | 197615561 |
| 197631460 | 197631510 | 197631560 | 197631610 | 197631660 |
| 197631588 | 197631638 | 197631688 | 197631738 | 197631788 |
| 197632490 | 197632540 | 197632590 | 197632640 | 197632690 |
| 197632606 | 197632656 | 197632706 | 197632756 | 197632806 |
| 197633270 | 197633320 | 197633370 | 197633420 | 197633470 |
| 197633760 | 197633810 | 197633860 | 197633910 | 197633960 |
| 197638678 | 197638728 | 197638778 | 197638828 | 197638878 |
| 197638849 | 197638899 | 197638949 | 197638999 | 197639049 |
| 197639280 | 197639330 | 197639380 | 197639430 | 197639480 |
| 197651923 | 197651973 | 197652023 | 197652073 | 197652123 |
| 197652378 | 197652428 | 197652478 | 197652528 | 197652578 |
| 197653025 | 197653075 | 197653125 | 197653175 | 197653225 |
| 197654442 | 197654492 | 197654542 | 197654592 | 197654642 |
| 197687170 | 197687220 | 197687270 | 197687320 | 197687370 |
| 197687424 | 197687474 | 197687524 | 197687574 | 197687624 |
| 197688112 | 197688162 | 197688212 | 197688262 | 197688312 |
| 197688345 | 197688395 | 197688445 | 197688495 | 197688545 |
| 197688392 | 197688442 | 197688492 | 197688542 | 197688592 |
| 197692891 | 197692941 | 197692991 | 197693041 | 197693091 |
| 197692896 | 197692946 | 197692996 | 197693046 | 197693096 |
| 197694165 | 197694215 | 197694265 | 197694315 | 197694365 |
| 197695268 | 197695318 | 197695368 | 197695418 | 197695468 |
| 197695491 | 197695541 | 197695591 | 197695641 | 197695691 |
| 197695757 | 197695807 | 197695857 | 197695907 | 197695957 |
| 197696092 | 197696142 | 197696192 | 197696242 | 197696292 |
| 197696632 | 197696682 | 197696732 | 197696782 | 197696832 |
| 197696662 | 197696712 | 197696762 | 197696812 | 197696862 |
| 197697227 | 197697277 | 197697327 | 197697377 | 197697427 |
| 197697414 | 197697464 | 197697514 | 197697564 | 197697614 |
| 197698149 | 197698199 | 197698249 | 197698299 | 197698349 |
| 197698178 | 197698228 | 197698278 | 197698328 | 197698378 |
| 197708037 | 197708087 | 197708137 | 197708187 | 197708237 |
| 197708234 | 197708284 | 197708334 | 197708384 | 197708434 |
| 197757973 | 197758023 | 197758073 | 197758123 | 197758173 |
| 197760075 | 197760125 | 197760175 | 197760225 | 197760275 |
| 197761154 | 197761204 | 197761254 | 197761304 | 197761354 |
| 197761205 | 197761255 | 197761305 | 197761355 | 197761405 |
| 197776440 | 197776490 | 197776540 | 197776590 | 197776640 |
| 197777449 | 197777499 | 197777549 | 197777599 | 197777649 |
| 197777518 | 197777568 | 197777618 | 197777668 | 197777718 |
| 197778010 | 197778060 | 197778110 | 197778160 | 197778210 |
| 197781749 | 197781799 | 197781849 | 197781899 | 197781949 |
| 197781861 | 197781911 | 197781961 | 197782011 | 197782061 |
| 197784596 | 197784646 | 197784696 | 197784746 | 197784796 |
| 197785066 | 197785116 | 197785166 | 197785216 | 197785266 |
| 197785170 | 197785220 | 197785270 | 197785320 | 197785370 |
| 197786048 | 197786098 | 197786148 | 197786198 | 197786248 |
| 197786055 | 197786105 | 197786155 | 197786205 | 197786255 |
| 197787668 | 197787718 | 197787768 | 197787818 | 197787868 |
| 197805956 | 197806006 | 197806056 | 197806106 | 197806156 |
| 197806383 | 197806433 | 197806483 | 197806533 | 197806583 |
| 197812495 | 197812545 | 197812595 | 197812645 | 197812695 |
| 197813489 | 197813539 | 197813589 | 197813639 | 197813689 |
| 197813982 | 197814032 | 197814082 | 197814132 | 197814182 |
| 197840702 | 197840752 | 197840802 | 197840852 | 197840902 |
| 197840851 | 197840901 | 197840951 | 197841001 | 197841051 |
| 197855889 | 197855939 | 197855989 | 197856039 | 197856089 |
| 197859223 | 197859273 | 197859323 | 197859373 | 197859423 |
| 197860611 | 197860661 | 197860711 | 197860761 | 197860811 |
| 197861273 | 197861323 | 197861373 | 197861423 | 197861473 |
| 197895172 | 197895222 | 197895272 | 197895322 | 197895372 |
| 197902723 | 197902773 | 197902823 | 197902873 | 197902923 |
| 197902755 | 197902805 | 197902855 | 197902905 | 197902955 |
| 197902823 | 197902873 | 197902923 | 197902973 | 197903023 |
| 197903019 | 197903069 | 197903119 | 197903169 | 197903219 |
| 197903164 | 197903214 | 197903264 | 197903314 | 197903364 |
| 197903202 | 197903252 | 197903302 | 197903352 | 197903402 |
| 197903272 | 197903322 | 197903372 | 197903422 | 197903472 |
| 197903375 | 197903425 | 197903475 | 197903525 | 197903575 |
| 197903487 | 197903537 | 197903587 | 197903637 | 197903687 |
| 197903529 | 197903579 | 197903629 | 197903679 | 197903729 |
| 197903616 | 197903666 | 197903716 | 197903766 | 197903816 |
| 197903716 | 197903766 | 197903816 | 197903866 | 197903916 |
| 197903916 | 197903966 | 197904016 | 197904066 | 197904116 |
| 197904555 | 197904605 | 197904655 | 197904705 | 197904755 |
| 197906573 | 197906623 | 197906673 | 197906723 | 197906773 |
| 197907466 | 197907516 | 197907566 | 197907616 | 197907666 |
| 197907517 | 197907567 | 197907617 | 197907667 | 197907717 |
| 197907553 | 197907603 | 197907653 | 197907703 | 197907753 |
| 197907742 | 197907792 | 197907842 | 197907892 | 197907942 |
| 197909724 | 197909774 | 197909824 | 197909874 | 197909924 |
| 197948446 | 197948496 | 197948546 | 197948596 | 197948646 |
| 197948480 | 197948530 | 197948580 | 197948630 | 197948680 |
| 197948590 | 197948640 | 197948690 | 197948740 | 197948790 |
| 197948731 | 197948781 | 197948831 | 197948881 | 197948931 |
| 197948779 | 197948829 | 197948879 | 197948929 | 197948979 |
| 197973532 | 197973582 | 197973632 | 197973682 | 197973732 |
| 197974393 | 197974443 | 197974493 | 197974543 | 197974593 |
| 197994108 | 197994158 | 197994208 | 197994258 | 197994308 |
| 198023332 | 198023382 | 198023432 | 198023482 | 198023532 |
| 198023473 | 198023523 | 198023573 | 198023623 | 198023673 |

23. The isolated polynucleic acid according to claim 18, comprising the restorer molecular marker allele, polymorphism, or SNP.

24. The isolated polynucleic acid according to claim 18, comprising the maintainer molecular marker allele, polymorphism, or SNP.

25. The isolated polynucleic acid according to claim 18, comprising at most 500 nucleotides, preferably at most 200 nucleotides, more preferably at most 100 nucleotides, most preferably at most 50 nucleotides, such as at most 35 nucleotides.

26. The isolated polynucleic acid according to claim 18, which is a primer or a probe.

27. The isolated polynucleotide according to claim 18, which is an allele-specific primer or probe, preferably a KASP primer.

28. A maize plant or plant part comprising one or more molecular marker allele of Table 4 or Table 5, the locus as defined in claim 1, and/or a polynucleic acid, wherein the polynucleic acid comprises:
(i) one or more molecular marker alleles of Table 4 or Table 5, or the complement or reverse complement of said molecular marker alleles, or
(ii) one or more of SEQ ID NOs: 1, 5, 9, and 13, or a fragment thereof, or a sequence which is at least 80%, preferably at least 85%, more preferably at least 90%, most preferably at least 95% identical to a sequence as set forth in any one or more of SEQ ID NOs: 1, 5, 9, and 13, or a fragment thereof; or one or more of SEQ ID NOs: 2, 6, 10, and 14, or a fragment thereof, or a sequence which is at least 80%, preferably at least 85%, more preferably at least 90%, most preferably at least 95% identical to a sequence as set forth in any one or more of SEQ ID NOs: 2, 6, 10, and 14, or a fragment thereof.

29. A method for generating a maize plant or plant part, comprising introducing in the genome of said plant or plant part a locus as defined in claim 1 or a polynucleic acid, or a functional fragment thereof, wherein the polynucleic acid comprises:
(i) one or more molecular marker alleles of Table 4 or Table 5, or the complement or reverse complement of said molecular marker alleles, or
(ii) one or more of SEQ ID NOs: 1, 5, 9, and 13, or a fragment thereof, or a sequence which is at least 80%, preferably at least 85%, more preferably at least 90%, most preferably at least 95% identical to a sequence as set forth in any one or more of SEQ ID NOs: 1, 5, 9, and 13, or a fragment thereof; or one or more of SEQ ID NOs: 2, 6, 10, and 14, or a fragment thereof, or a sequence which is at least 80%, preferably at least 85%, more preferably at least 90%, most preferably at least 95% identical to a sequence as set forth in any one or more of SEQ ID NOs: 2, 6, 10, and 14, or a fragment thereof.

30. The method according to claim 29, comprising introducing in the genome of said plant or plant part the locus or the polynucleic acid, or a functional fragment thereof, wherein the polynucleic acid comprises:
(i) a restorer molecular marker allele, polymorphism, or SNP of Table 4 or Table 5, or
(ii) a maintainer molecular marker allele, polymorphism, or SNP of Table 4 or Table 5.

31. The method according to claim 29, wherein said polynucleic acid is a genomic polynucleic acid flanked by molecular markers ma0016fm86 and ma0004tr23.

32. The method according to claim 29, wherein said polynucleic acid is a genomic polynucleic acid flanked by molecular markers ma0000sa77 and ma0016fu05.

33. The method according to claim 29, wherein said locus or polynucleic acid comprises the maintainer polymorphisms of Table 5.

34. The method according to claim 29, wherein said locus or polynucleic acid comprises the maintainer polymorphisms of Table 4.

35. The method according to claim 29, wherein said locus or polynucleic acid comprises the restorer polymorphisms of Table 5.

36. The method according to claim 29, wherein said locus or polynucleic acid comprises the restorer polymorphisms of Table 4.

37. The method according to claim 30, comprising crossing a first maize plant and a second maize plant, and selecting offspring comprising the locus or the polynucleic acid.

38. The method according to claim 37, comprising selecting offspring not comprising the restorer locus, and/or not comprising the polynucleic acid comprising the restorer polymorphisms or SNPs.

39. The method according to claim 37, wherein said first or second maize plant is a cytoplasmic male sterile maize plant.

40. A method of using a polynucleic acid according to claim 18 for identifying a maize plant or plant part or for generating a maize plant or plant part.

41. A method of using a polynucleic acid according to claim 18 for identifying a maize plant or plant part.

42. A method of using a polynucleic acid according to claim 19 for generating a maize plant or plant part.
